(19) United States
(12) Patent Application Publication
(10) Pub. No.: US 2024/0325557 A1
(43) Pub. Date: Oct. 3, 2024

(54) TREATMENT OF CANCER

(71) Applicant: INNATE PHARMA, MARSEILLE (FR)

(72) Inventors: MANEL KRAIEM, MARSEILLE (FR); STÉPHANIE CHANTEUX, MARSEILLE (FR); BENJAMIN ROSSI, MARSEILLE (FR)

(21) Appl. No.: 18/038,723

(22) PCT Filed: Nov. 24, 2021
(86) PCT No.: PCT/EP2021/082872

§ 371 Date: May 25, 2023

Related U.S. Application Data
(60) Provisional application No. 63/229,589, filed on Aug. 5, 2021.
(60) Provisional application No. 63/118,198, filed on Nov. 25, 2020.

(51) Int. Cl. A61K 47/68; A61P 35/00

(52) U.S. Cl. CPC A61K 47/68037; A61K 47/6849; A61K 47/6855; A61K 47/6889; A61P 35/00

(57) ABSTRACT

The invention relates to antigen-binding proteins capable of binding to Nectin-4 polypeptides conjugated to chemotherapeutic agents, for use in increasing sensitivity of tumors to the chemotherapeutic agents and for use in the treatment of cancers characterized by Nectin-4-expressing tumor cells.

### CROSS-REFERENCE TO RELATED APPLICATIONS

This application is the U.S. National Stage Application of International Patent Application No. PCT/EP2021/082872, filed on Nov. 24, 2021, which claims the benefit of U.S. Provisional Application No. 63/118,198 filed 25 Nov. 2020 and U.S. Provisional Application No. 63/229,589 filed 5 Aug. 2021; which are incorporated herein by reference in their entireties; including any drawings.

### REFERENCE TO SEQUENCE LISTING

The present application is being filed along with a Sequence Listing in electronic format. The Sequence Listing is provided as a file entitled “Seq-List-replace.txt”, created Jan. 24, 2024, which is 68,091 bytes in size. The information in the electronic format of the Sequence Listing is incorporated herein by reference in its entirety.

### FIELD OF THE INVENTION

The present invention provides antigen-binding proteins capable of binding to Nectin-4 polypeptides conjugated to chemotherapeutic agents, for use in increasing sensitivity of tumors to the chemotherapeutic agents, for use in the treatment of cancers characterized by Nectin-4-expressing tumor cells.

### BACKGROUND OF THE INVENTION

In the United States in 2016, it was estimated that there were over 70,000 new diagnoses of bladder cancer, with about 16,000 deaths. Urothelial cancers encompass carcinomas of the bladder, ureters, and renal pelvis, which occur at a ratio of 50:3:1, respectively. Cancer of the urothelium is a multifocal process. Patients with cancer of the upper urinary tract have a 30% to 50% chance of developing cancer of the bladder at some point in their lives. Bladder cancer occurs when the cells in the bladder start to grow unusually or uncontrollably. The most common type of bladder cancer is called urothelial carcinoma (UC). In UC, unusual growth takes place on the inside lining (urothelium) of the bladder. As the disease progresses, it may spread. It can spread to the areas around the bladder or to other parts of the body (metastasis). This is called advanced urothelial carcinoma.

Urothelial carcinoma (UC) is characterized by increased expression of a range of different cell surface antigens, thus offering opportunities for specific therapeutic targeting with use of antibody-drug conjugates (ADCs). Among the surface antigens, several antigens have shown to be amenable to development of ADCs, including TROP-2 (human trophoblast cell-surface antigen), SLITRK family proteins (e.g. SLITRK6), EpCAM, HER2, TF-Ag (Thomsen-Friedrich antigen), FGF1V, Fn14 (FGF-inducible 14), PSMA (prostate-specific membrane antigen) and Nectin-4 (poliovirus receptor-related protein 4, also known as PVRL4.

Nectin-4 was initially closed from human trachea by the Lopez group in 2001 (see Reymond et al. (2001) J. Biol. Chem. 276 (46): 43205-15). It has been reported that copy number gain of the Nectin-4 gene is a frequent event in carcinogenesis and further that it can promote epithelial-to-mesenchymal transition, invasion and metastasis. While nectin-4 protein expression is limited in healthy tissues, is expressed at significantly higher levels in several tumors, and it is particularly over-expressed in breast cancer, including triple negative breast cancer (TNBC) (See M-Rabet et al. Ann Oncol. 2017 Apr. 1;28 (4): 769-776), pancreatic cancer and in UC. However, nectin-4 is also expressed in non-small cell lung cancer, ovarian cancer, head and neck squamous cell carcinoma and esophageal cancer tumor specimens. Challita-Eid et al. (2016) Cancer Res. 76 (10): 3003-3013 reported that moderate to strong staining by immunohistochemistry (H-score≥100) in bladder (60%) and breast (53%) tumor tissues. Zeindler et al. 2019 Front. Med. 6:200 reported that a high expression of Nectin-4 was present in 86 (58%) of the 148 TNBC cases.

Challita-Eid et al. (2016), supra, developed an anti-Nectin-4 antibody conjugated to the highly potent microtubule-disrupting agent MMAE based on antibody AGS-22. The work gave rise to the ADC drug candidate enfortumab vedotin (see U.S. Pat. No. 8,637,642 and PCT publication No. WO2012/047724, Agensys Inc.) which has yielded promising results in human clinical trials in treatment of patients with locally advanced or metastatic urothelial cancer who have previously received a platinum-containing chemotherapy in the neoadjuvant/adjuvant, locally advanced, or metastatic setting and a PD-1/PD-L1 checkpoint inhibitor. Several other groups have also proposed anti-Nectin-4 agents bound to a variety of toxic agents. PCT patent application WO2018/158398 (INSERM) reports several anti-Nectin-4 antibodies and proposes potential coupling to a range of cytotoxic agents. Similarly, U.S. Pat. No. 8,637,642 (Agensys Inc.) also provides anti-Nectin-4 antibodies and proposes potential coupling to a range of cytotoxic agents. Yet further, Bicycle Therapeutics has reported development of an anti-Nectin-4 targeting agent comprised of a Nectin-4 binding protein conjugated to a cytotoxic auristatin (MMAE) payload via a valine-citrulline (val-cit), cleavable linker. To date, all the anti-Nectin-4 antibodies that have been reported as active when conjugated to chemotherapeutic agents have targeted the Ig-like V type domain of Nectin-4, consequently the Ig-like V type domain is believed to provides the strongest internalization of anti-Nectin-4 ADCs.

Enfortumab vedotin (anti-Nectin-4 ADC) binds to the Ig-like V type domain of Nectin-4 which is associated with highly-internalizing anti-Nectin-4 antibodies. Enfortumab vedotin has shown impressive therapeutic responses with an ORR (objective response rate) of 44% and CR (complete response rate) of 12% in UC in the EV-201 Phase 2 study (2019), about half of the patients discontinued treatment. Most of the discontinuation was due to progressive disease as assessed by RECIST (48%) or clinical symptoms (5%). Also, 18% patients that discontinued experienced adverse events, notably neuropathy. The Nectin-4-targeted ADCs therefore have limitations, and there is a need in the art for improved benefit to patients afflicted with UC and other cancers.

In UC, individuals are generally first treated with cisplatin-based regimens (with or without radiation) both when the cancer has not spread to distant parts of the body, or when the cancer has spread. Camptothecin compounds are generally not used in the treatment of UC. De Jonge et al., 2004 Invest New Drugs 22:329-333 studied the camptothecin analogue RFS2000 in patients with advanced or metastatic urothelial tract tumors. De Jonge et al. 2004 concluded that it did not exert significant activity in patients with advanced/metastatic urothelial tract tumors failing prior chemotherapy, and that the results of this study do not suggest further investigation of RFS2000. A great number of camptothecin analogues have been made over the last few decades, among them exatecan. Abou-Alfa et al., 2006 Journal of Clinical Oncology 24 (27): 4441-4447 evaluated exatecan in the treatment of pancreatic cancer in a phase 3 trial of 349 patients, finding that exatecan in addition to gemcitabine was not superior in efficacy to gemcitabine alone.

### SUMMARY OF THE INVENTION

Many highly potent cytotoxic agents such as pyrrolobenzodiazepine and auristatins have been developed for conjugation to antibodies, however at the doses used in treatment, ADCs incorporating these agents still cause serious side effects due to the toxicity of the agent. Other cytotoxic agents such as camptothecins are less toxic but also have limited anti-tumor activity on their own. Their anti-tumor activity of cytotoxic agents can be enhanced by conjugation to anti-Nectin-4 antibodies, however as demonstrated herein, when conjugated to gold-standard anti-Nectin-4 antibodies such as enfortumab (ASG-22), camptothecin analogue-based ADCs still have limitations when tested in vivo. The inventors provide herein methods for improved delivery of cytotoxic agents to Nectin-4-positive tumors. In particular, the present application shows that a strong anti-tumor effect can be obtained by conjugating a cytotoxic agent to an antibody that binds the domain or region of Nectin-4 bridging the Ig-like V and Ig-like C2 type 1 domains. The domain bridging the Ig-like V and Ig-like C2 type 1 domains, also referred to herein as the VC1 bridging domain, the VC1 determinant or the VC1 epitope, is associated with cell-to-cell interactions. Targeting this domain with an ADC that displays decreased binding to Nectin-4 mutants having VC1 bridging domain amino acid substitutions (at residues K197 and/or S199, as exemplified by mutants comprising the K197T/S199A double substitution) compared to wild-type or non-mutated Nectin-4 resulted in a 3-fold higher potency in anti-tumor activity compared to gold-standard anti-Ig-like V type domain ADCs that have high intracellular internalization potential. It is possible that the cell-to-cell interactions are mediated via the interaction of Nectin-4 on a first cell with Nectin-1 and/or Nectin-4 on a second cell (Nectin-4: Nectin-1 or Nectin-4: Nectin-4 interactions).

Yet further, the present application shows that a particularly strong anti-tumor effect can be obtained in cancers characterized by tumor having high Pgp expression by functionalizing an antibody that binds the VC-bridging domain with an intracellularly-cleavable linker that releases exatecan upon cleavage of the linker. The anti-Nectin-4-exatecan ADC permits an effective anti-tumor effect in the MDR-high tumor model which is resistant to the same antibody that instead releases the camptothecin analogue Dxd.

In some aspects, provided is an antibody (e.g. a full-length antibody, an antibody fragment) that specifically binds a human Nectin-4 polypeptide and is capable of sensitizing a tumor to a cytotoxic agent (or for example a protein comprising such antibody), for use in a method for treating cancer and/or inhibiting anticancer drug resistance. Optionally, the antibody that binds a human Nectin-4 polypeptide is used (administered) in combination with a cytotoxic agent (e.g., a chemotherapeutic agent).

One object of the present disclosure is to provide an anti-cancer composition comprising the cancer-sensitizing composition and an anticancer agent (e.g. a cytotoxic agent, a chemotherapeutic agent), wherein the cancer-sensitizing composition comprises an antibody (e.g. a full-length antibody, an antibody fragment) that binds the VC1 bridging domain of a Nectin-4 polypeptide.

In some aspects, provided is an antibody (e.g. a full-length antibody, an antibody fragment) that binds a human Nectin-4 polypeptide and is capable of sensitizing a tumor to a cytotoxic agent, for use in preparation of an antibody drug conjugate.

In some aspects, provided are improved methods of delivering cytotoxic agents (e.g. a camptothecin, an exatecan) to tumors (e.g. tumor cells within solid tumors), by conjugating the cytotoxic agent to an antibody (e.g. a full-length antibody, an antibody fragment) that binds the VC1 bridging domain of Nectin-4.

In any embodiment herein relating to immunoconjugate or ADCs, the cytotoxic agent can be conjugated or bound to an antibody via a linker comprising an intracellularly cleavable moiety (e.g. an oligopeptide, a di-, tri- or tetra-peptide) and optionally a self-eliminating spacer. In any embodiment, the cytotoxic agent is conjugated or bound to an antibody via a linker comprising an intracellularly cleavable moiety and optionally a self-eliminating spacer, resulting in release of the cytotoxic agent (e.g. a camptothecin analogue, an exatecan, a compound having the structure of Compounds 1, 2 or 13) upon cleavage of the cleavable moiety, e.g., in a tumor or tumor cell.

The improved delivery of the cytotoxic agent and/or the sensitization to a chemotherapeutic or cytotoxic agent may be the result of antibody-mediated inhibition of cluster formation and/or anchorage-independent growth of Nectin-4 expressing tumor cells, resulting in improved tumor penetration of the ADC. 10

According to one aspect of the present invention, provided is a sensitizer-cytotoxic drug conjugate, optionally for use in the treatment of cancer, wherein the sensitizer-cytotoxic drug conjugate comprises: (a) at least one antigen binding domain that binds to the VC1 bridging domain of human Nectin-4, optionally wherein the antigen binding domain comprises (i) a heavy chain variable region comprising an amino acid sequence that has at least 70%, 80%, 85%, 90%, 95% or 98% identity with an amino acid sequence having the Kabat heavy chain CDR1, 2 and 3 of antibody 5E7, 10B12, 6C11B or 6A7 and human framework regions (e.g. from human V gene (IGHV) group(s) disclosed herein), and/or (ii) a light chain variable region comprising an amino acid sequence that has at least 70%, 80%, 85%, 90%, 95% or 98% identity with an amino acid sequence having the Kabat light chain CDR1, 2 and 3 of antibody 5E7, 10B12, 6C11B or 6A7 and human framework regions (e.g. from human V gene (IGKV) group(s) disclosed herein), and (b) at least one cytotoxic drug moiety. The polypeptides (i) and (ii) can be specified to associate with one another (e.g. by covalent and/or non-covalent bonds) to form an antigen binding domain that binds to Nectin-4.

In one aspect, the ADCs incorporating the antibodies of the disclosure provide enhanced potency, and are therefore particularly advantageous for the treatment of disease with lower Nectin-4 expression (e.g. less than high expression) or heterogeneous Nectin-4 expression (e.g. urothelial cancer), for patients having particularly resistant disease. In some embodiments, this permits treatment independent of or without the assessment or detection of levels of Nectin-4 expression in tumors prior to treatment.

In one aspect, the ADCs incorporating the antibodies of the disclosure provide enhanced tumor penetration of ADCs, which can improve anti-tumor activity, reduce the amount of ADC required and/or reduce toxicity related to the cytotoxic agent. As a result of this improved therapeutic window compared to gold-standard internalizing anti-Nectin-4 antibodies (e.g. Ig V type domain antibodies), the ADCs of the disclosure are advantageous for treatment of patients having disease resistant to other treatments, for whom conventional ADCs are not suitable (e.g. due to toxicity), and/or for use in combination with additional therapeutic agents that as single agents that mediate their own toxicity (e.g., chemotherapeutic agents alone or as incorporated into an ADC).

In some aspects, provided is an antibody (e.g. a full-length antibody, an antibody fragment) that specifically binds Nectin-4, wherein the antibody binds to the VC1 bridging domain of human Nectin-4.

In some aspects, provided is an antibody (e.g. a full-length antibody, an antibody fragment) that specifically binds Nectin-4, wherein the antibody displays reduced binding to a mutant human Nectin-4 polypeptide comprising an amino acid substitution at residues K197 and/or S199 (with reference to SEQ ID NO: 1), compared to binding to a wild-type human Nectin-4 polypeptide.

In some aspects, provided is an antibody (e.g. a full-length antibody, an antibody fragment) comprising the heavy and light chains CDRs of antibody 5E7, 10B12, 6C11B or 6A7.

In any aspect, the antibody of the disclosure is for use in preparation of an antibody drug conjugate. In any aspect, the antibody of the disclosure is for use in (e.g., for use in a method of) reducing cell-cell adhesion between Nectin-4 expressing tumor cells, inhibiting a Nectin-4: Nectin-1 interaction, inhibiting a Nectin-4: Nectin-4 interaction, reducing growth of Nectin-4 expressing tumor cells and/or reducing cluster formation of Nectin-4 expressing tumor cells.

In some aspects, provided is an antibody (e.g. a full-length antibody, an antibody fragment) that binds a human Nectin-4 polypeptide and is capable of reducing cell-cell adhesion between Nectin-4 expressing tumor cells, and/or reducing growth and/or cluster formation of Nectin-4 expressing tumor cells (as assessed, for example, using 3-dimensional or non-adherent tumor cell culture; tumor spheroid assays), for use in preparation of an antibody drug conjugate.

In some aspects, provided is an antibody (e.g. a full-length antibody, an antibody fragment) that is capable of inhibiting a Nectin-4: Nectin-1 interaction and/or a Nectin-4: Nectin-4 interaction (e.g., the antibody is capable of reducing the interaction of Nectin-4 on a first cell with Nectin-1 and/or Nectin-4 on a second cell, optionally wherein the cell(s) is a tumor cell).

In some aspects, preparation of an antibody drug conjugate comprises a step of conjugating the antibody to a cytotoxic agent (e.g. via a linker moiety, a linker moiety further comprising an intracellularly cleavable moiety).

In some aspects, provided are methods of preparing an antibody drug conjugate comprising conjugating an anti-Nectin-4 antibody according to the disclosure to a cytotoxic agent.

Also provided is an antibody drug conjugate comprising the anti-Nectin-4 antibody according to conjugated to a cytotoxic agent.

Also provided are methods of treating a cancer comprising administering an antibody drug conjugate according to comprising the anti-Nectin-4 antibody conjugated to a cytotoxic agent.

The increased anti-tumor potency and greater therapeutic window offered by an anti-Nectin-4 antibody of the disclosure (e.g., particularly when conjugated to a camptothecin derivative) provides the possibility for improved treatment outcomes in individuals having tumors having resistance to, that are not responsive to or that have progressed following treatment with a composition comprising an anti-HER2 agent (e.g. trastuzumab, an ADC comprising trastuzumab). The improved therapeutic window provides the possibility for combination treatment with other agents, particularly chemotherapeutic agents and/or anti-HER2 agents.

In some aspects, provided is a Nectin-4 binding agent conjugated to a cytotoxic agent, optionally a chemotherapeutic agent, as well as use thereof in the treatment of a Nectin-4-expressing cancer, for example UC, breast cancer (e.g. TNBC), non-small cell lung cancer, pancreatic cancer, ovarian cancer, head and neck squamous cell carcinoma or an esophageal cancer, wherein the binding agent is an antibody that binds a human Nectin-4 polypeptide and is capable of sensitizing tumors or tumor cells, particularly metastatic cancers, to a cytotoxic agent.

In some aspects, provided is a Nectin-4 binding agent conjugated to a cytotoxic agent, optionally a chemotherapeutic agent, as well as use thereof in the treatment of Nectin-4-expressing cancers, for example UC, breast cancer (e.g. TNBC), non-small cell lung cancer, pancreatic cancer, ovarian cancer, head and neck squamous cell carcinoma or an esophageal cancer, wherein the binding agent is an antibody that binds the VC1 bridging domain of human Nectin-4; e.g., to a stretch of amino acids bridging the Ig-like C2 type 1 domain and the Ig-like V type domain, to an epitope positioned at least partly within the amino acids bridging the Ig-like C2 type 1 domain and the Ig-like V type domain.

The feature of binding to the VC1 bridging domain of Nectin-4 can optionally be characterized as the ability to bind (e.g. at least partly) to (a) a human Nectin-4 polypeptide Ig-like V type domain and (b) to a human Nectin-4 polypeptide Ig-like C2 type 1 domain. In another aspect, the feature of binding to the VC1 bridging domain of Nectin-4 can optionally be characterized as retaining partial binding to a Nectin-4 polypeptide in which the Ig-like V type domain has been deleted (e.g. a Nectin-4 polypeptide comprising the Ig-like C2 type 1 domain and the Ig-like C2 type 2 domain, but lacking the Ig-like V type domain; a polypeptide of SEQ ID NO: 32), optionally wherein the antibody retains binding to the Nectin-4 protein with deleted Ig-like V type domain at a reduced level compared to binding to a wild-type Nectin-4 protein); optionally further wherein the anti-Nectin-4 antibody does not bind a Nectin-4 polypeptide lacking the Ig-like V type and the Ig-like C2 type 1 domains, e.g., a polypeptide having the amino acid sequence of SEQ ID NO: 33. In another aspect, the feature of binding to the VC1 bridging domain of Nectin-4 can be optionally be characterized as displaying reduced binding to a mutant Nectin-4 polypeptide comprising one or more amino acid substitutions at residues A72, G73, S195, K197, S199, L150, S152, Q234 and 1236 (with reference to SEQ ID NO: 1), optionally a mutant Nectin-4 polypeptide comprising one or more amino acid substitutions at residues K197, S199 and/or Q234, compared to binding to a wild-type human Nectin-4 polypeptide, optionally a mutant Nectin-4 polypeptide comprising one or more amino acid substitutions at residues K197 and/or S199, compared to binding to a wild-type human Nectin-4 polypeptide. Optionally, binding is assessed by flow cytometry using cells made to express the Nectin-4 polypeptide and fluorescence intensity level (e.g. MFI) is determined.

In one embodiment, provided is a Nectin-4 binding antibody or antibody fragment that is a function-conservative variant of antibody 5E7, 10B12, 6C11B or 6A7. In one embodiment, a Nectin-4 binding antibody comprises: (i) a VH domain comprising an amino acid sequence that has at least 70%, 80%, 85%, 90%, 95% or 98% identity with the amino acid sequence defined by a sequence selected from the group comprising SEQ ID NOS: 6, 24, 42 or 50 (optionally wherein each heavy chain framework region is substituted by a human heavy chain framework region), and/or (ii) a VL domain comprising an amino acid sequence that has at least 70%, 80%, 85%, 90%, 95% or 98% identity with the amino acid sequence defined by a sequence selected from the group comprising SEQ ID NOS: 7, 25, 43 or 51 (optionally wherein each light chain framework region is substituted by a human light chain framework region).

In one embodiment, provided is a Nectin-4 binding antibody or antibody fragment comprising: (i) a heavy chain variable region comprising the amino acid sequences of SEQ ID NOS: 8, 9 and 10 (Kabat H-CDRs 1, 2 and 3) and (ii) a light chain variable region comprising the amino acid sequences of SEQ ID NO: 11, 12 and 13 (Kabat L-CDRs 1, 2 and 3).

In one embodiment, provided is a Nectin-4 binding antibody or antibody fragment comprising: (i) a heavy chain variable region comprising the amino acid sequences of SEQ ID NOS: 14, 15 and 16 (H-CDRs 1, 2 and 3) and (ii) a light chain variable region comprising the amino acid sequences of SEQ ID NO: 17, 18 and 13 (L-CDRs 1, 2 and 3).

In one embodiment, provided is a Nectin-4 binding antibody or antibody fragment comprising: (i) a heavy chain variable region comprising the amino acid sequences of SEQ ID NOS: 19, 20 and 21 (H-CDRs 1, 2 and 3) and (ii) a light chain variable region comprising the amino acid sequences of SEQ ID NOS: 22, 18 and 23 (L-CDRs 1, 2 and 3).

In one embodiment, provided is a Nectin-4 binding antibody or antibody fragment comprising: (i) a heavy chain variable region comprising the amino acid sequences of SEQ ID NOS: 44, 45 and 46 (H-CDRs 1, 2 and 3) and (ii) a light chain variable region comprising the amino acid sequences of SEQ ID NOS: 47, 48 and 49 (L-CDRs 1, 2 and 3).

In one embodiment, provided is a Nectin-4 binding antibody or antibody fragment comprising: (i) a heavy chain variable region comprising the amino acid sequences of SEQ ID NOS: 52, 53 and 54 (H-CDRs 1, 2 and 3) and (ii) a light chain variable region comprising the amino acid sequences of SEQ ID NOS: 55, 56 and 57 (L-CDRs 1, 2 and 3).

In any embodiment herein, the anti-Nectin-4 antibody or antibody fragment, or the antibody-drug conjugate comprising such antibody or fragment, is capable, upon binding to Nectin-4 on the surface of a tumor cell, of undergoing intracellular internalization.

In any embodiment herein, the anti-Nectin-4 antibody is conjugated to a cytotoxic agent. In any embodiment herein, the anti-Nectin-4 antibody conjugated to a cytotoxic agent is used in combination with a further cytotoxic agent (e.g. chemotherapeutic agent, a chemotherapeutic agent transported by P-glycoprotein (Pgp, the product of the human MDR1 gene), a platinum agent, a taxane), wherein the further cytotoxic agent is administered separately from the anti-Nectin-4 antibody conjugated to a cytotoxic agent.

In one aspect, provided is a method of treating a cancer and/or killing tumor cells in an individual in need thereof, comprising administering to said individual a therapeutically effective amount of an antibody of the disclosure that specifically binds to a human Nectin-4 polypeptide, optionally wherein the antibody is conjugated via a linker to a cytotoxic agent, optionally a camptothecin analogue.

In one aspect, the present invention provides methods of treatment that can be used in individuals having a Nectin-4-expressing cancer irrespective of the level of Nectin-4 expression on tumor cells.

In one aspect, the present invention provides methods of treatment that can be used advantageously in individuals whose tumor cells express P-glycoprotein (Pgp).

In one aspect, the present invention provides methods of treatment that can be used advantageously in individuals having received prior treatment with a chemotherapeutic agent (e.g. a chemotherapeutic agent transported by P-glycoprotein (Pgp), a platinum agent (e.g., oxaliplatin, cisplatin, carboplatin, nedaplatin, Phenanthriplatin, picoplatin, satraplatin), a taxane (e.g., Paclitaxel (Taxol™) and docetaxel (Taxotere™).

In one aspect, the present invention provides methods of treatment that can be used advantageously in an individual whose tumor or cancer has resistance, that is not responsive to or that has progressed following treatment with a composition comprising an anti-HER2 antibody (e.g. trastuzumab, an ADC comprising the heavy and light variable regions, CDRs or polypeptide chains trastuzumab).

In one aspect, the present invention provides methods of treatment that can be used to mediate an anti-tumor effect in an individual at doses that are low or lower than those employed for conventional anti-Nectin-4 ADCs, e.g. less than 3 mg/kg body weight, at less than 1.25 mg/kg body weight, less than 1 mg/kg body weight, less than 125 mg flat dose.

In one aspect, the present invention provides methods of treatment that can be used in an individual who has existing neuropathy, diabetes or hyperglycemia, cardiac insufficiency, an ocular pathology.

In one aspect, the present invention provides methods of treatment that can be used in an individual having a Nectin-4-expressing cancer characterized by low or moderate levels of tumor cell expression of Nectin-4 polypeptides (e.g. expression of Nectin-4 polypeptides at the tumor cell membrane).

In one aspect, the anti-Nectin-4 antibody or antibody fragment is conjugated to a cytotoxic agent via an intracellularly-cleavable (e.g., protease cleavable) oligo-peptide (e.g. di-, tri, tetra- or penta-peptide). In one aspect, the anti-Nectin-4 antibody or antibody fragment is conjugated to a cytotoxic agent (e.g. a camptothecin derivative) via an intracellularly-cleavable (e.g. protease-cleavable) di-, tri-, tetra- or penta-peptide and a self-eliminating spacer. In one aspect, the anti-Nectin-4 antibody or antibody fragment is conjugated to a cytotoxic agent (e.g. a camptothecin derivative) via an Entracellularly-cleavable (e.g., protease cleavable) tetra- or penta-peptide and a self- or non-self-eliminating spacer.

In one aspect, the cytotoxic agent is a highly potent chemotherapeutic agent, optionally a cytotoxic agent selected from the group consisting of taxanes, anthracyclines, camptothecins, epothilones, mytomycins, combretastatins, vinca alkaloids, nitrogen mustards, maytansinoids, duocarmycins, tubulysins, dolastatins and auristatins, enediynes (e.g., calicheamicins, esperamicins, shishijimicins and namenamicins), pyrrolobenzodiazepines (e.g. pyrrolobenzodiazepine dimers and indolino-pyrrolobenzodiazepine dimers), amatoxins and ethylenimines. In one embodiment, the cytotoxic agent is a DNA damaging agent, include for example a DNA intercalating agent, e.g. an agent that inserts itself into the DNA structure of a cell and binds to the DNA, in turn causing DNA damage (e.g. daunorubicin). Compounds include topoisomerase inhibitors, chemical compounds that block the action of topoisomerase (topoisomerase I and II). Such compounds are used for a wide range of solid tumor and hematological malignancies, notably lymphomas. Topoisomerase I inhibitors include camptothecins, for example irinotecan (approved for treatment of colon cancer), topotecan (approved for treatment of ovarian and lung cancer), camptothecin, lamellarin D, indenoisoquinoline, indimitecan. Further camptothecins include silatecan, cositecan, exatecan, lurtotecan, gimatecan, belotecan, and rubitecan. Topoisomerase II inhibitors include for example etoposide (VP-16), teniposide, doxorubicin, daunorubicin, mitoxantrone, amsacrine, ellipticines, aurintricarboxylic acid, and HU-331, a quinolone synthesized from cannabidiol.

Optionally the antibody is functionalized with a linker-toxin of any one of Formulae I to XI.

In one aspect, the cytotoxic agent is camptothecin analogue, e.g., an exatecan, Dxd or SN-38 molecule.

In one aspect, the present invention provides a Nectin-4 binding antibody or antibody fragment of the disclosure conjugated (e.g. covalently bound to) to a camptothecin, e.g. a camptothecin analogue, an exatecan or exatecan derivative, a Dxd molecule or a SN-38 molecule.

In any embodiment herein, a Nectin-4 binding antibody or antibody fragment of the disclosure conjugated (e.g. covalently bound to) to a camptothecin analogue, e.g. an exatecan or SN-38 molecule, can be characterized as comprising an antibody that specifically binds to a human Nectin-4 polypeptide having one or more amino acid residues (e.g. cysteine, lysine, glutamine residues, a non-natural amino acid residue) functionalized, via a linker, with a molecule comprising the structure of Compounds 1 or 2, or functionalized with the linker-camptothecin molecule of Formulas I or II. In any embodiment herein, a Nectin-4 antibody or antibody fragment can be characterized as being functionalized with a linker-camptothecin molecule having a structure of Formulas III, IV, V, VI, VII, VII, IX, X or XI, or with any of Compounds 3 to 12.

In one embodiment, the Nectin-4 antibody or antibody fragment conjugated to a camptothecin derivative is a Nectin-4 antibody or antibody fragment conjugated to an exatecan molecule, e.g. a molecule having the structure of Compound 1 (1a or 1b). In any embodiment, a cleavable linker or an immunoconjugate or ADC comprising a linker can be characterized as releasing an exatecan molecule, e.g. a molecule having the structure of Compound 1 (1a or 1b), e.g. upon enzymatic cleavage of the linker. In one embodiment, the Nectin-4 antibody or antibody fragment conjugated to a camptothecin derivative is a Nectin-4 antibody or antibody fragment conjugated to a SN-38 molecule, e.g., a molecule having the structure of Compound 2. In one embodiment, the Nectin-4 antibody or antibody fragment can be characterized as comprising an antibody that specifically binds to a human Nectin-4 polypeptide having one or more amino acid residues (e.g. cysteine, lysine, glutamine or non-natural amino acid residues) functionalized, via a linker (e.g. a cleavable linker molecule with or without an additional spacer, for example spacer (Y′) described herein), with a molecule having the structure:

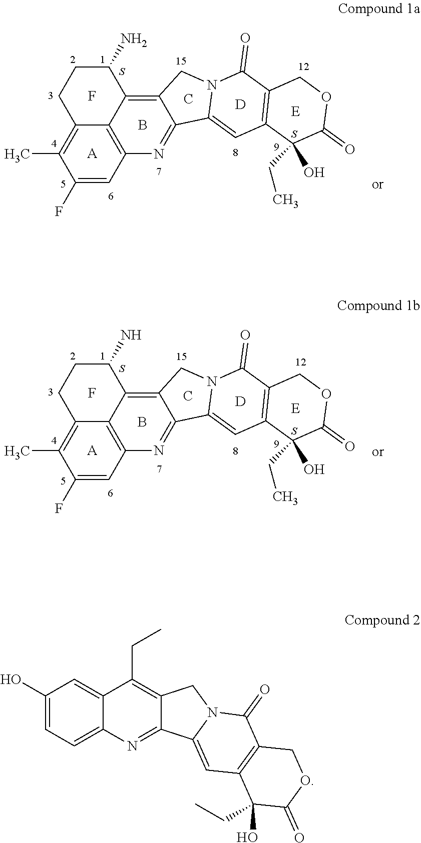

In one embodiment, the Nectin-4 antibody or antibody fragment conjugated to a cytotoxic agent can be specified as being an immunoconjugate represented by Formula (I):

Ab-X—Z Formula (I)
wherein,
Ab is an antibody or antibody fragment that specifically binds to a human Nectin-4 polypeptide (e.g. an antibody or antibody fragment that binds the VC1 bridging domain of human Nectin-4; and/or that displays decreased binding to a mutant human Nectin-4 polypeptide comprising an amino acid substitution at residues K197 and/or S199 (with reference to SEQ ID NO: 1), compared to binding to a wild-type human Nectin-4 polypeptide);
X is a linker molecule which connects Ab and Z (e.g., is covalently bound to each of Ab and Z), wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety; and
Z is a cytotoxic agent, optionally wherein Z is a camptothecin analogue, optionally wherein Z is exatecan, optionally wherein Z is exatecan and cleavage of the linker results in the release of a compound having the structure of Compound 1 (exatecan).

In one embodiment, the Nectin-4 antibody or antibody fragment conjugated to a cytotoxic agent can be specified as being an immunoconjugate represented by Formula (I):

Ab-X—Z Formula (I)
wherein,
Ab is an antibody or antibody fragment according to the disclosure that specifically binds to a human Nectin-4 polypeptide;
X is a linker molecule which connects Ab and Z (e.g., is covalently bound to each of Ab and Z), wherein X comprises a valine-citrulline, valine-alanine or phenylalanine-lysine dipeptide, wherein X further comprises a self-eliminating or non-self-eliminating spacer system (Y′) positioned between the cleavable moiety and Z, and wherein X further comprises a spacer (Y) positioned between the Ab and the cleavable moiety; and

Z is a cytotoxic agent, optionally a camptothecin analogue, optionally an exatecan molecule or a SN-38 molecule.

In one embodiment, provided is a method of delivering or targeting a cytotoxic agent (optionally a camptothecin analogue) to a tumor, a method of releasing a cytotoxic agent (optionally a camptothecin analogue, a Dxd, an exatecan) in a tumor (e.g. in a subject having cancer), or a method of sensitizing a tumor or cancer to a cytotoxic agent (optionally a camptothecin analogue), the method comprising administering to a subject having a cancer an immunoconjugate represented by Formula (I):

Ab-X—Z Formula (I)
wherein,
Ab is an antibody or antibody fragment that specifically binds to a human Nectin-4 polypeptide;
X is a linker molecule which connects Ab and Z (e.g., is covalently bound to each of Ab and Z), wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety; and
Z is a cytotoxic agent, optionally a camptothecin analogue, optionally an exatecan molecule or a SN-38 molecule, optionally wherein Z is exatecan and wherein cleavage of the linker results in the release of a compound having the structure of Compound 1 (exatecan).

In one embodiment, the antibody or antibody fragment conjugated to a cytotoxic agent (optionally a camptothecin analogue) can be specified as being an immunoconjugate represented by Formula (II):

Ab-(X—(Z)ₙ)ₘ Formula (II)
wherein,
Ab is an antibody or antibody fragment according to the disclosure that specifically binds to a human Nectin-4 polypeptide;
X is a linker molecule which connects Ab and Z, wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety;
Z is a cytotoxic agent, optionally a camptothecin analogue, optionally Z is a molecule comprising an exatecan molecule or a SN-38 molecule, e.g., a molecule having the structure of Compounds 1 or 2;
n is 1; and
m is from 4 to 8, or optionally m is an integer selected from among 4, 5, 6, 7 or 8. In one embodiment, a Nectin-4 antibody or antibody fragment conjugated to a cytotoxic agent, (optionally a camptothecin analogue) can be characterized as a composition of immunoconjugates represented by Formula (II):

Ab-(X—(Z)ₙ)ₘ Formula (II)
wherein,
Ab is an antibody or antibody fragment according to the disclosure that specifically binds to a human Nectin-4 polypeptide;
X is a molecule which connects Ab and Z, wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety;
Z is a cytotoxic agent, optionally a camptothecin analogue, optionally Z is a molecule comprising an exatecan molecule or a SN-38 molecule;
wherein n is 1, and at least 50%, 60%, 70%, 80%, 90%, 95%, 98% or 99% of immunoconjugates in the composition have an m (the number of X—Z moieties) that is between 2 and 4, between 4 and 8, optionally between 6 and 8. Optionally at least 50%, 60%, 70%, 80%, 90%, 95%, 98% or 99% of immunoconjugates in the composition have an m that is, or that is at least, 4, 6, 7 or 8.

In Formulae I or II, the (X—Z) moiety can optionally be characterized having a structure of any of Formulae III to XI or of any of Compounds 3-12.

In Formulae I or II, molecule X or spacer Y can optionally be specified as comprising a reactive group € or a residue of the reaction of a reactive group € with an amino acid of the antigen binding protein (e.g., antibody) or with a complementary reactive group (R′) that is attached to an amino acid of the antibody or antibody fragment.

In any embodiment herein, the exatecan molecule can be specified as being bound to linker (X) via the amine at position 1 of the exatecan (NH replaces NH2 at position 1 when the exatecan molecule is part of a linker). In one embodiment, the exatecan is bound to the linker (X), e.g. to the carbonyl of a p-aminobenzyloxycarbonyl (PAB) self-eliminating spacer moiety of X. In any embodiment herein, the SN-38 molecule can specified as being bound to linker (X) via the OH at position 9 (O replaces OH at position 9 when the SN-38 molecule shown in Compound 2 is part of a linker).

In one embodiment, the Nectin-4 binding agent conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan molecule comprising the following structure:

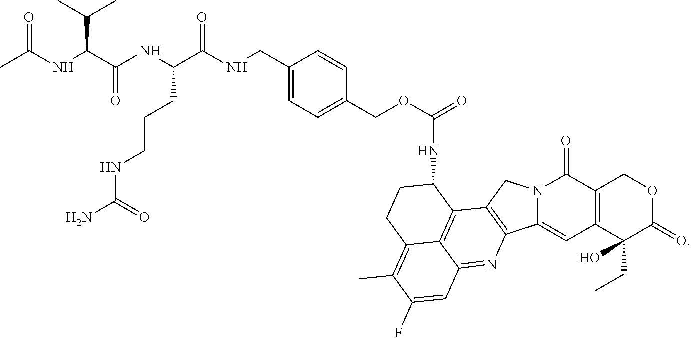 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(C)=O)C(C)C)cc1)CC3]

In one embodiment, the Nectin-4 antibody or antibody fragment conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan comprising the following structure:

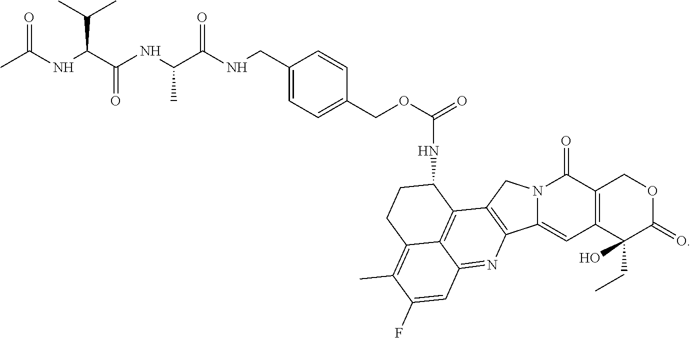 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](C)NC(=O)[C@@H](NC(C)=O)C(C)C)cc1)CC3]

In one embodiment, the Nectin-4 antibody or antibody fragment conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan molecule comprising the following structure:

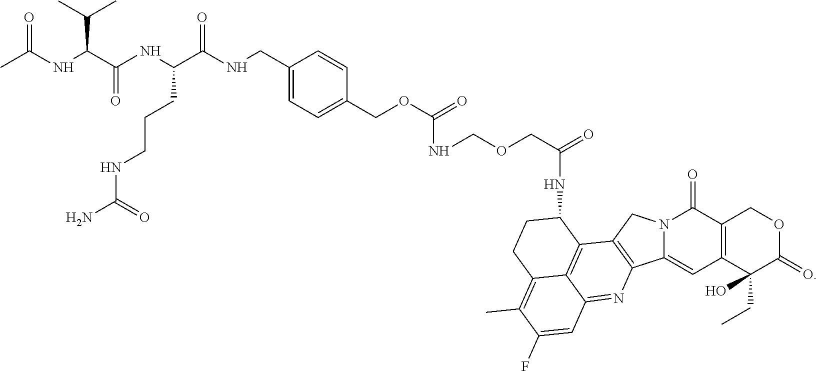 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(CNC(=O)[C@H](CCCNC(N)=O)NC(=O)[C@@H](NC(C)=O)C(C)C)cc1)CC3]

In one embodiment, the Nectin-4 antibody or antibody fragment conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan molecule comprising the following structure:

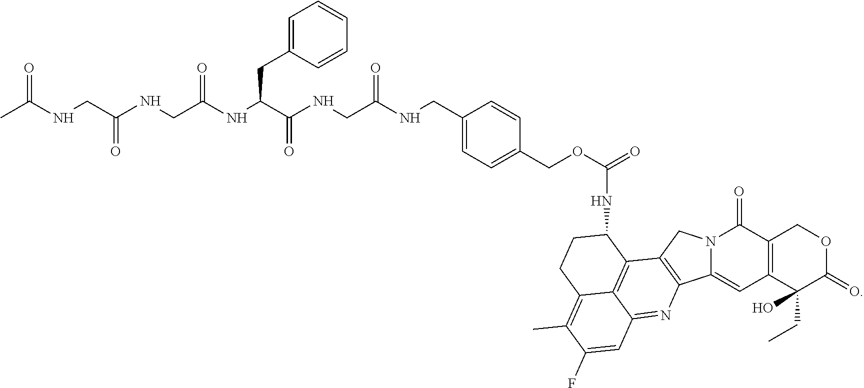 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)CNC(=O)[C@H](Cc2ccccc2)NC(=O)CNC(=O)CNC(C)=O)cc1)CC3]

Spacer (Y) can be specified as being or comprising a substituted or unsubstituted alkyl or heteroalkyl chain, optionally wherein Y has a chain length of 2-40 atoms, optionally 2-30, 2-20, 4-40, 4-30 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide. For example Y may comprise one or more ethylene oxide monomers, optionally Y comprises a polyethylene oxide moiety, optionally Y comprises a structure —(CH₂CH₂O)ₓ— where x is 1 to 12, optionally 1 to 8, optionally 1 to 6.

In one aspect, the present invention provides a treatment showing improved efficacy and/or improved (lower) drug resistance compared to existing anti-Nectin-4 ADC therapies (e.g. an anti-Nectin-4 antibody or antibody fragment conjugated to an auristatin; enfortumab verdotin). In one aspect, provided is a method of treating and/or preventing a cancer and/or killing tumor cells in an individual in need thereof, wherein the treatment comprises 2, 3, 4, 5, 6, 7, 8, 9 or 10 or more administrations of a Nectin-4 binding agent conjugated to a camptothecin derivative (e.g. an exatecan or SN-38 molecule) at a frequency of 1 to 2 times per month (e.g. once every two weeks, once every three weeks or once every four weeks).

In one aspect, the present invention provides a method of treating and/or preventing a cancer and/or killing tumor cells in an individual in need thereof, comprising treating said individual with Nectin-4 binding antibody or antibody fragment conjugated to a camptothecin molecule (e.g., an anti-Nectin-4 antibody or antibody fragment conjugated to one or more camptothecin moieties), optionally wherein the camptothecin is an exatecan, Dxd or SN-38. In one embodiment, said individual has a Nectin-4-expressing tumor, optionally wherein the tumor is an HER2-expressing tumor or a HER2-negative tumor, e.g. an urothelial cancer, a breast cancer, a non-small cell lung cancer, pancreatic cancer, ovarian cancer, head and neck squamous cell carcinoma or an esophageal cancer. In one embodiment, said individual has a Pgp-expressing tumor. In one embodiment, the cancer or tumor is advanced recurrent or metastatic cancer, optionally an advanced recurrent or metastatic urothelial cancer. In one embodiment, the cancer or tumor is a triple-negative breast cancer (TNBC).

In one aspect, the present invention provides a method of treating a cancer, killing tumor cells and/or delivering a cytotoxic agent to a tumor, in an individual in need thereof, the method comprising administering to said individual a therapeutically effective amount of an antibody-drug conjugate comprising an anti-Nectin-4 antibody conjugated via a cleavable linker to an exatecan of Compound 1, optionally wherein cleavage of the linker results in the release of a compound having the structure of Compound 1 (exatecan), optionally wherein the cleavable linker comprises a cleavable oligopeptide (e.g. di-, tri- or tetra-peptide, val-cit, val-phe, val-ala) and a self-immolating spacer (e.g. PAB), and wherein said individual has cancer that has relapsed and/or that has progressed following treatment with a binding agent (optionally a Nectin-4-binding agent) conjugated via a linker to a cytotoxic agent capable of being transported by Pgp (e.g. the linker is an intracellularly cleavable linker that releases the cytotoxic agent upon cleavage). In any embodiment, the cytotoxic agent capable of being transported by Pgp is a camptothecin analogue other than exatecan, optionally wherein the cytotoxic agent capable of being transported by Pgp is a five-ring camptothecin analogue, a six-ring camptothecin, a deruxtecan or a Dxd, optionally wherein cleavage of the linker results in the release of the camptothecin analogue other than exatecan (e.g., Dxd, having the structure of Compound 13). In one embodiment, the binding agent conjugated via a linker to a cytotoxic agent capable of being transported by Pgp comprises an antibody that binds Nectin-4 conjugated to a compound having the structure of Compound 13 (Dxd, deruxtecan) via a linker, optionally wherein the linker comprises a cleavable glycine-containing oligopeptide linker, for example glycine- and phenylalanine-containing oligopeptide linker, an GGFG linker. In one embodiment, the anti-Nectin-4 antibody conjugated via a cleavable linker to an exatecan of Compound 1 comprises an antibody that binds to the VC1 bridging domain of a human Nectin-4 polypeptide, e.g. an antibody of the disclosure.

In one aspect of any embodiment herein, the individual has received prior treatment with radiotherapy, surgery, chemotherapy, and/or treatment with a biological agent.

Provided also are compositions of immunoconjugates of the disclosure. Provided also are pharmaceutically acceptable compositions and kits comprising the immunoconjugates of the disclosure, and typically one or more additional ingredients that can be active ingredients or inactive ingredients that promote formulation, delivery, stability, or other characteristics of the composition (e.g., various carriers). Provided also are methods of screening, testing and making anti-Nectin-4 antibodies and antibody fragments, and immunoconjugates or ADC that comprise them, as well as tools and reagents for use therein.

These aspects are more fully described in, and additional aspects, features, and advantages will be apparent from, the description of the invention provided herein.

### DESCRIPTION OF THE DRAWINGS

FIG. 1 shows expression levels of HER2 and Nectin-4 polypeptides at the surface of SUM 190 human breast cancer tumor cells, as determined by FACS (MFI: Mean of fluorescence intensity). The SUM190 tumor cells expressed HER2 at low to moderate levels (median fluorescence units 1777) as well as Nectin-4 at lower levels (median 991 fluorescence units).

FIG. 2 shows expression levels of HER2 and Nectin-4 polypeptides at the surface of SUM 185 human breast cancer tumor cells as determined by FACS (MFI: Mean of fluorescence intensity). The SUM185 cells expressed HER2 at moderate to high levels (median fluorescence units 2880) as well as Nectin-4 at higher levels (median 4326 fluorescence units).

FIG. 3, right hand panels, show efficacy of N4 ADC1 (anti-Nectin-4) in causing the death of HER-2 and Nectin-4 expressing SUM185 and SUM190 tumor cells. The two left hand panels of shows the efficacy of the HER2 ADC1 (anti-Her2) in the same respective cells. N4 ADC1 had a 20-fold increase in potency compared to HER2 ADC1 in the SUM190 cells, as well as higher comparative potency even in the SUM185 cells characterized by lower Nectin-4 surface expression (about 4-fold lower surface Nectin-4 in SUM185 than in SUM 190).

FIG. 4A shows MFI for binding of anti-Nectin-4 antibodies to whole (wild-type) Nectin-4 protein, as assessed by flow cytometry using cell made to express Nectin-4. mAbs 6 (5E7) and 7 (10B12) show a lower plateau that other antibodies.

FIG. 4B shows MFI for binding of anti-Nectin-4 antibodies to cells made to express a modified Nectin-4 having the Ig-like C2 type 1 and the Ig-like C2 type 2 domains and lacking the Ig-like V domain (the C1C2 construct), assessed by flow cytometry. The left hand panel shows that mAbs 6 (5E7) and 7 (10B12) retain binding to the C1C2 construct. The right hand panel. The right hand panel shows that the MFI for mAbs6 and 7 is reduced compared to mAb2 which binds fully to the C1C2 construct.

FIG. 5 shows the domain structure of Nectin-4. Antibodies enfortumab and N41 used as ADCs bind to the Ig-like V type domain. Antibodies 1-5 obtained herein bind to the Ig-like C2 type 1 domain (C1 domain) and/or to the Ig-like C2 type 2 domain (C2 domain) but not to the Ig-like V type domain. Antibodies 6 (5E7) and 7 (10B12), and antibodies 6CB11 and 6A7 obtained herein bind to both the Ig-like V type domain (V domain) and to the Ig-like C2 type 1 domain (C1 domain).

FIG. 6 shows the binding profile of the different antibodies 1-7 and isotype control (IC) on SUM 185 cells, as assessed by flow cytometry. From this experiment, the C1 and/or C2 domain specific antibodies (mAbs1-5) generally appear to have a binding profile comparable to enfortumab, while antibodies 6 and 7 (mAbs6 and 7) have a different profile.

FIG. 7A shows internalization on SUM190 cells, as luminescence (indicating cell viability) vs. anti-Nectin-4 antibody concentration, for each of antibodies 1-7 compared to enfortumab. FIG. 7B shows luminescence (indicating cell viability) for mAb6 compared to enfortumab on SUM185 cells. mAb6 (5E7) was more efficient than enfortumab in inducing internalization in the SUM185 cells.

FIG. 8A shows killing of human breast cancer cells by the mAb6 (5E7) antibody conjugated with the camptothecin analogues Dxd (via a GGFG-Dxd linker shown in Example 7) or exatecan (via the (PEG (8U)-Val-Ala-PAB-Exatecan) linker shown in Example 7), along with isotype control antibodies (IC), all at equivalent drug to antibody ratios (DAR=8), and compared to compared to V-domain binding Enhertu™ (trastuzumab deruxtecan (anti-HER2)).

FIG. 8B shows killing of human breast cancer cells by the mab6 (5E7) conjugated with the same camptothecin analogue-comprising linkers of FIG. 8A, along with immunocontrol conjugated with the camptothecin analogues, mAb3 conjugated to camptothecin analogue-comprising linkers at the same DAR, and Enhertu™. FIG. 8C shows efficacy of “5E7-exatecan” (5E7 conjugated to the exatecan linker (PEG (8U)-Val-Ala-PAB-Exatecan) shown in Example 7) as in causing the death of HER-2 and Nectin-4 expressing SUM185, SUM190, MDA-MB-468 (TNBC) human tumor cells and MC38 (colon cancer) and B16F10 (melanoma) murine tumor cells as well as EC₅₀ values for ability of 5E7-exatecan to cause cell death.

FIG. 9 shows the efficacy at a single dose of 3 mg/kg of mAb6 (5E7) antibody as a camptothecin ADC, in comparison to the same dose of enfortumab, N41 and an isotype control antibody (IC), in a mouse model of human breast cancer. Each antibody was conjugated with the same camptothecin analogue (Dxd) and tetrapeptide-containing linker (GGFG) at equivalent drug to antibody ratios (DAR=8). Only 5E7 was able to effectively control tumor growth.

FIG. 10 shows killing of SUM 185 human breast cancer cells that express Nectin-4 at relatively high levels by the mAb6 (5E7) antibody, compared to Padcev™ (enfortumab verdotin) (enfortumab conjugated to the auristatin compound MMAE at DAR=4) and Enhertu™. This setting is used as a model of anti-HER2 resistance.

FIGS. 11A and 11B show binding of anti-Nectin-4 antibodies on rat and cynomolgus Nectin 4 expressing CHO cell lines respectively, as determined by flow cytometry. Enfortumab and mAbs 6 and 7 bind to rat Nectin-4, while mAbs1-5 do not.

FIGS. 12A and 12B show the structure of the human Nectin-4 protein, with shading of substitutions; the white areas corresponding to the C1 domain residues substituted in mutants 7 (12A) and 7bis (12B) are indicated.

FIGS. 13A and 13B show several views of the structure of the human Nectin-4 protein, with shading of substitutions; the white areas corresponding to residues substituted in mutants 1, 2, 3, 4, 5, 6, 7, 8 and 9 are indicated.

FIG. 14A shows luminescence (indicating cell viability) for 6C11B compared to antibody 5E7 on SUM190 cells. FIG. 14B shows luminescence (indicating cell viability) for 6A7 compared to antibody 5E7 on SUM190 cells. Both 6C11B and 6A7 were each as efficient as 5E7 in inducing internalization in the SUM190 cells.

FIGS. 15A, 15B and 15C show the evolution of tumor growth over time (days post tumor engraftment) in mice treated with ADCs FIGS. 15A and 15B show treatment with a dose of 3 mg/kg body weight by i.v. of different antibodies conjugated to the same linker-payload at the same DAR. FIG. 15C shows treatment at a dose of 10 mg/kg body weight by i.v. with antibody 5E7 conjugated to linkers designed release either Dxd or exatecan upon cleavage of the linker.

FIG. 16A shows luminescence (indicating cell viability) of cells treated with Padcev™ (enfortumab vedotin), antibody 5E7 conjugated to Dxd or 5E7 conjugated to exatecan. The cells are MC-38 cells that endogenously express MDR1 p-glycoprotein and that are engineered to express Nectin-4. The ADC with exatecan as payload was highly potent to decrease cell viability in this setting of drug resistance. FIG. 16B shows tumor growth (area under the curve) of the MC38 cells treated, in the presence or absence of the Pgp inhibitor cyclosporine, with Padcev™ (enfortumab vedotin), antibody 5E7 conjugated to Dxd or 5E7 conjugated to exatecan, at 150 nM ADC and normalized to control antibody, suggesting that the anti-tumor activity of Padcev™ and antibody 5E7 conjugated to Dxd are negatively affected by Pgp.

FIGS. 17A and 17B show the evolution of tumor growth over time in mice treated with enfortumab vedotin, antibody 6A7 conjugated to deruxtecan or 6A7 conjugated to exatecan, injected once (FIG. 17A) or in three weekly injections (FIG. 17B), in a model of MDR1 p-glycoprotein-expressing tumors. Antibody 6A7 conjugated to exatecan was able to induce a significant reduction of tumor volume in this model of drug resistance.

### DETAILED DESCRIPTION

### Introduction

The inventors provide herein methods for improved delivery of cytotoxic agents to Nectin-4-positive tumors. In particular, the present invention arises, inter alia, from observations that certain antibodies that bind to a site in the region of Nectin-4 bridging the Ig-like V and Ig-like C2 type 1 domains which is associated with cell-to-cell interactions results in a 3-fold higher potency in anti-tumor activity compared to gold-standard anti-Ig-like V type domain ADCs. The cell-to-cell interactions may be mediated via the interaction of Nectin-4 on a first cell with Nectin-1 and/or Nectin-4 on a second cell (Nectin-4: Nectin-1 or Nectin-4: Nectin-4 interactions. When prepared as an ADC bearing a topoisomerase I inhibitor (camptothecin analogues Dxd and exatecan), the antibodies that bound to the aforementioned site on Nectin-4 displayed greater anti-tumor activity in vivo when compared to gold-standard antibody enfortumab or a comparator antibody that bond to a different area on the Nectin-4 bridging the Ig-like V and Ig-like C2 type 1 domain.

Generally, the antibodies and fragments that can optionally be employed for the ability to sensitize (or in a method of sensitizing) a tumor to a cytotoxic agent and/or for the ability to inhibit (or in a method of inhibiting) anticancer drug resistance. The antibodies and fragments of the disclosure can thus be used (administered) in combination with a cytotoxic agent (e.g., a chemotherapeutic agent), where in the cytotoxic agent is administered as a non-conjugated agent administered separately or in the same pharmaceutical formulation as the anti-Nectin-4 antibody, or as an immunoconjugate where the cytotoxic agent is conjugated to the anti-Nectin-4 antibody.

One object of the disclosure is an antibody (e.g. a full-length antibody, an antibody fragment) that binds to an epitope on Nectin-4 comprising one, two or three resides selected from the group consisting of S195, K197 and S199 (with reference to SEQ ID NO: 1).

One object of the disclosure is an antibody (e.g. a full-length antibody, an antibody fragment) having a reduced binding to a mutant Nectin-4 polypeptide comprising a mutation at one or more (or all of) residues selected from the group consisting of S195, K197 and S199 (with reference to SEQ ID NO: 1), optionally, the mutant Nectin-4 polypeptide has the mutations S195A, K197T and S199A.

One object of the disclosure is an antibody (e.g. a full-length antibody, an antibody fragment) having a reduced binding to a mutant Nectin-4 polypeptide comprising a mutation at one or more (or all of) residues selected from the group consisting of S195, K197 and S199 (with reference to SEQ ID NO: 1), optionally, the mutant Nectin-4 polypeptide has the mutations S195A, K197T and S199A, for use in preparation of an antibody drug conjugate.

One object of the disclosure is a human Nectin-4 polypeptide comprising a mutation at one or more (or all of) residues selected from the group consisting of S195, K197 and S199 (with reference to SEQ ID NO: 1). The mutant human Nectin-4 polypeptide can be a full length Nectin-4 polypeptide or a fragment thereof, for example an extracellular domain fragment, a VC domain fragment, a fragment comprising the V and C1 domains or a fragment comprising at least 10, 20, 30, 50. 100 or 200 contiguous amino acid residues of a Nectin-4 polypeptide of SEQ ID NO: 1. Optionally, the mutant Nectin-4 polypeptide comprises mutations at residues K197 and S199 (e.g. K197T and S199A), optionally, the mutant Nectin-4 polypeptide comprises mutations at residues S195, K197 and S199 (e.g., S195A, K197T and S199A), optionally, the mutant Nectin-4 polypeptide comprises mutations at residues A72, G73, K197 and S199 (e.g. A72P/G73N/K197T/S199A). Also provided are recombinant host cells that express (are made to express) such polypeptide, e.g. at the cell surface. The mutant polypeptide and/or host cells can be used in methods of making, testing, preparation or screening of an antibody and/or an antibody drug conjugate.

Reduced binding to a mutant Nectin-4 polypeptide can be as compared to binding to a Nectin-4 polypeptide without the mutation, e.g. a wild-type Nectin-4 polypeptide such as the polypeptide comprising the amino acid sequence of SEQ ID NO: 1.

One object of the disclosure is a Nectin-4 binding antibody or antibody fragment that is a function-conservative variant of antibody 5E7, 10B12 or 6A7. One object of the disclosure is a Nectin-4 binding antibody or antibody fragment that competes with antibody 5E7, 10B12 or 6A7 for binding to an epitope on Nectin-4. One object of the disclosure is a Nectin-4 binding antibody or antibody fragment that binds the same site or epitope on Nectin-4 as antibody 5E7, 10B12 or 6A7. One object of the disclosure is an antibody or antibody fragment comprising the heavy and light chains CDRs of antibody 5E7, 10B12 or 6A7. One object of the disclosure is an antibody or antibody fragment that binds Nectin-4 and that comprises heavy and light chains CDRs that have at least 70%, 80%, 85%, 90%, 95% or 98% sequence identity to the heavy and light chains CDRs of antibody 5E7, 10B12 or 6A7.

One object of the disclosure is a method of making an ADC. When preparing ADCs, it will be advantageous to link a cytotoxic agent (e.g. a topoisomerase inhibitor or camptothecin analogue) to an antibody that binds the aforementioned site on Nectin-4. In one object, provided is a method for making an ADC, the method comprising conjugating a cytotoxic agent (Z) to an anti-Nectin-4 antibody that binds to an epitope on Nectin-4 comprising one, two or three resides selected from the group consisting of S195, K197 and S199 and/or that has reduced binding to a mutant Nectin-4 polypeptide having the mutations S195A, K197T and S199A). Optionally, the method comprises contacting and/or reacting said anti-Nectin-4 antibody with a cytotoxic agent (Z) under conditions suitable such that an antibody drug conjugate is formed, and isolating the antibody drug conjugate, optionally further wherein the anti-Nectin-4 antibody is conjugated to the cytotoxic agent via a linker (X) which connects the antibody (Ab) and cytotoxic agent (Z), optionally further, wherein the cytotoxic agent (Z) is a topoisomerase I inhibitor, a camptothecin, optionally a five-ring camptothecin analogue, optionally a six-ring camptothecin analogue, optionally exatecan, SN-38 or Dxd.

One object of the disclosure is an ADC comprising an Nectin-4 binding antibody or antibody fragment described herein conjugated to a cytotoxic agent, optionally further wherein the cytotoxic agent is a topoisomerase I inhibitor, optionally a camptothecin analogue. When prepared as an ADC bearing a topoisomerase I inhibitor (camptothecin analogues Dxd and exatecan), the antibodies that bound to the aforementioned site on Nectin-4 were highly potent in the ability to kill cancer cell lines expressing both Nectin-4 and P-glycoprotein, notably in HCT116, A375, MC38, A549, HT29 and MDA-MB-231 cell lines. Accordingly, such ADCs having efficacy in cancer multi-drug resistance can be used advantageous in treatment of Nectin-4 expressing cancer that are resistant to other drugs (e.g. auristatins, MMAE). One object of the disclosure is use of immunoconjugates of the disclosure to treat a cancer, optionally a urothelial cancer, optionally an advanced recurrent or a metastatic urothelial cancer, a breast cancer, a TNBC, a non-small cell lung cancer, a pancreatic cancer, an ovarian cancer, a head and neck squamous cell carcinoma or an esophageal cancer. One object of the disclosure is use of immunoconjugates of the disclosure to treat a cancer in an individual, wherein said individual has relapsed and/or that has progressed following treatment with a Nectin-4-binding agent conjugated to an auristatin or to a cytotoxic agent capable of being transported by Pgp, optionally wherein the Nectin-4-binding agent conjugated to an auristatin is enfortumab verdotin. One object of the disclosure is use of immunoconjugates of the disclosure to treat a cancer in an individual wherein said individual has relapsed and/or that has progressed following treatment with an antibody that binds to HER2, optionally an antibody that binds to HER2 conjugated to a cytotoxic agent, optionally an agent that binds HER2 conjugated to a camptothecin analogue, optionally wherein the antibody comprises the CDRs and/or variable regions of trastuzumab. In one aspect, the individual has a Nectin-4-expressing cancer characterized by low or moderate Nectin-4 expression on tumor cells, optionally as determined by immunohistochemistry. In one aspect, the individual has a Nectin-4- and HER2-expressing cancer. In one aspect, the individual has a cancer characterized by tumor cells that express at their surface low or moderate levels of HER2 protein. In one aspect, the individual has a cancer characterized by tumor cells that express at their surface high levels of HER2 protein. In one aspect, the individual has an urothelial cancer, optionally an advanced recurrent or a metastatic urothelial cancer, a breast cancer, a TNBC, a non-small cell lung cancer, a pancreatic cancer, an ovarian cancer, a head and neck squamous cell carcinoma or an esophageal cancer. In one aspect, the individual has received and/or has a cancer that has progressed following prior treatment with a chemotherapeutic agent, optionally a chemotherapeutic agent transported by P-glycoprotein (Pgp), a platinum agent or a taxane.

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

“Nectin-4” and “Nectin-4 polypeptide” refer to a protein or polypeptide encoded by the NECTIN4 gene (see Uniprot accession number Q96NY8) or by a cDNA prepared from such a gene. Any naturally occurring isoform, allele or variant is encompassed by the term Nectin-4 polypeptide (e.g., an Nectin-4 polypeptide 95%, 98% or 99% identical to SEQ ID NO: 1, or to a contiguous sequence of at least 100, 200, 300, 400 or 500 amino acid residues thereof). The 510 amino acid residue sequence of canonical human Nectin-4 (isoform 1), including the 31 amino acid signal peptide, is shown as follows:

(SEQ ID NO: 1)
MPLSLGAEMW GPEAWLLLLL LLASFTGRCP AGELETSDVV TVVLGQDAKL
PCFYRGDSGE QVGQVAWARV DAGEGAQELA LLHSKYGLHV SPAYEGRVEQ
PPPPRNPLDG SVLLRNAVQA DEGEYECRVS TFPAGSFQAR LRLRVLVPPL
PSLNPGPALE EGQGLTLAAS CTAEGSPAPS VTWDTEVKGT TSSRSFKHSR
SAAVTSEFHL VPSRSMNGQP LTCVVSHPGL LQDQRITHIL HVSFLAEASV
RGLEDQNLWH IGREGAMLKC LSEGQPPPSY NWTRLDGPLP SGVRVDGDTL
GFPPLTTEHS GIYVCHVSNE FSSRDSQVTV DVLDPQEDSG KQVDLVSASV
VVVGVIAALL FCLLVVVVVL MSRYHRRKAQ QMTQKYEEEL TLTRENSIRR
LHSHHTDPRS QPEESVGLRA EGHPDSLKDN SSCSVMSEEP EGRSYSTLTT
VREIETQTEL LSPGSGRAEE EEDQDEGIKQ AMNHFVQENG TLRAKPTGNG
IYINGRGHLV.

SEQ ID NO: 1 corresponds to UniProt KB identifier Q96NY8-1, the disclosure of which is incorporated herein by reference.

Certain aspects of the present disclosure provide anti-Nectin-4 antibodies that bind to a human Nectin-4, or a homolog thereof, including without limitation a mammalian Nectin-4 protein and Nectin-4 orthologs from other species, e.g. non-human primates, Macaca fascicularis.

The term “HER2” (also known as HER2/neu and ErbB-2) stands for “Human Epidermal growth factor Receptor 2”. It includes variants and isoforms of HER2.

The terms “immunoconjugate” and “antibody conjugate” are used interchangeably and refer to an antigen binding agent, e.g., an antibody binding polypeptide or an antibody that is conjugated to another molecule (e.g., a camptothecin derivative, an exatecan molecule, a SN-38 molecule). When an immunoconjugate comprises an antigen binding agent conjugated to a therapeutic agent, e.g., a cytotoxic agent or anti-cancer agent, the immunoconjugate can also be referred to as a “antibody drug conjugate” or an “ADC”. Examples of cytotoxic agents include camptothecins, taxanes, anthracyclines, camptothecins, epothilones, mytomycins, combretastatins, vinca alkaloids, nitrogen mustards, maytansinoids, calicheamycins, duocarmycins, tubulysins, dolastatins and auristatins, enediynes, pyrrolobenzodiazepines, and ethylenimines.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human” and incl “des: (a) preventing the disease from occurring in a subject which may be predisposed to the disease but has not yet been diagnosed as having it such as a preventive early asymptomatic intervention; (b) inhibiting the disease, e.g., arresting its development; or relieving the disease, e.g., causing regression of the disease and/or its symptoms or conditions such as improvement or remediation of damage, for example in a subject who has been diagnosed as having the disease. Optionally, treatment may cause (e.g. may be characterized as a method of causing) a decrease in tumor burden, a decrease in the size and/or number of lesions, a decrease or delay in the progression of cancer (e.g., an increase in progression-free survival), a delay or prevention of cancer metastasis and/or an increase in survival. Optionally, treatment may cause or provide (e.g. may be characterized as a method of causing or providing) stable disease, a partial response or a complete response in a subject, e.g. according to standard criteria, optionally RECIST criteria.

Whenever “treatment of cancer” or the like is mentioned with reference to a Nectin-4 binding agent (e.g. antibody or antibody fragment), are comprised:
(a) a method of treatment of cancer, said method comprising the step of administering (for at least one treatment) a Nectin-4 binding agent to an individual, a mammal, especially a human, in need of such treatment, in a dose that allows for the treatment of cancer, (a therapeutically effective amount), optionally in a dose (amount) as specified herein;
(b) the use of a Nectin-4 binding agent for the treatment of cancer;
€ the Nectin-4 binding agent, for use in the treatment of cancer (especially in a human);
(d) the use of a Nectin-4 binding agent for the manufacture of a pharmaceutical preparation for the treatment of cancer;
€ a method of using a Nectin-4 binding agent for the manufacture of a pharmaceutical preparation for the treatment of cancer, comprising admixing a Nectin-4 binding agent with a pharmaceutically acceptable carrier;
(f) a pharmaceutical preparation comprising an effective dose of a Nectin-4 binding agent that is appropriate for the treatment of cancer;
(g) any combination of (a), (b), (c), (d), € and (f), in accordance with the subject matter allowable for patenting in a country where this application is filed.

The term “biopsy” as used herein is defined as removal of a tissue for the purpose of examination, such as to establish diagnosis. Examples of types of biopsies include by application of suction, such as through a needle attached to a syringe; by instrumental removal of a fragment of tissue; by removal with appropriate instruments through an endoscope; by surgical excision, such as of the whole lesion; and the like.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The amino acid residues of an antibody that are responsible for antigen binding can also be referred to hypervariable region. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “specifically binds to” means that an antibody can bind preferably in a competitive binding assay to the binding partner, e.g. Nectin-4, as assessed using either recombinant forms of the proteins, epitopes therein, or native proteins present on the surface of isolated target cells. Competitive binding assays and other methods for determining specific binding are well-known in the art. For example, binding can be detected via radiolabels, physical methods such as mass spectrometry, or direct or indirect fluorescent labels detected using, e.g., cytofluorometric analysis (e.g. FACScan). Binding above the amount seen with a control, non-specific agent indicates that the agent binds to the target.

When an antibody is said to “compete with” a particular monoclonal antibody, it means that the antibody competes with the monoclonal antibody in a binding assay using either recombinant molecules (e.g., Nectin-4) or surface expressed molecules (e.g., Nectin-4). For example, if a test antibody reduces the binding of an antibody having a heavy chain variable region of any of SEQ ID NOS: 6 or 24 and a respective light chain variable region of SEQ ID NO: 7 or 25 to a Nectin-4 polypeptide or Nectin-4-expressing cell in a binding assay, the antibody is said to “compete” respectively with such antibody.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of monoclonal antibodies can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of monoclonal antibodies is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “agent” is used herein to denote a chemical compound, a mixture of chemical compounds, a biological macromolecule, or an extract made from biological materials. The term “therapeutic agent” refers to an agent that has biological activity.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to Refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Enformation (NCBI) and other sources (BLAST Manual, Altschul et al. NCB/NLM/NIH Bethesda, Md. 20894; Altschul et al., supra). The well-known Smith Waterman algorithm may also be used to determine identity.

As used herein, “alkyl” refers to a straight or branched hydrocarbon chain that comprises a fully saturated (no double or triple bonds) hydrocarbon group. The alkyl group may have, for example, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, the term “heteroalkyl” refers to a straight or branched alkyl group that contains one or more heteroatoms, that is, an element other than carbon (including but not limited to oxygen, sulfur, nitrogen, phosphorus) in place of one or more carbon atoms.

Whenever a group is described as being “substituted” that group substituted with one or more of the indicated substituents. If no substituents are indicated, it is meant that the indicated “substituted” group may be substituted with one or more group(s) individually and independently selected from alkyl, alkenyl, alkynyl, cycloalkyl, cycloalkenyl, cycloalkynyl, heteroalkyl, aryl, heteroaryl, heteroalicyclyl, aralkyl, heteroaralkyl, (heteroalicyclyl)alkyl, hydroxy, alkoxy, aryloxy, acyl, mercapto, alkylthio, arylthio, cyano, halogen, thiocarbonyl, carbamyl, thiocarbamyl, amido, sulfonamido, sulfonamido, carboxy, isocyanato, thiocyanato, isothiocyanato, nitro, silyl, sulfinyl, sulfenyl, sulfonyl, haloalkyl, haloalkoxy, trihalomethanesulfonyl, trihalomethanesulfonamido, an amino, a mono-substituted amino group and a di-substituted amino group, and protected derivatives thereof.

Where the number of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

Reference to a “Compound” or “Formula” having a particular number (e.g. “Compound 1”, “Compound 2”, “Formula I” or “Formula II”), unless the context clearly indicates otherwise, designates all compounds derived from the Compound or Formula having the particular number. Compound 1, for example includes reference to Compound 1a and 1b.

### Nectin-4 Binding Antibodies and Antibody Fragments

The hypervariable regions, heavy and light chain CDRs, heavy and light chain variable regions, and antibodies, e.g., full-length antibodies or antibody fragments, that comprise them, will bind human Nectin-4 expressed on the surface of a cell, e.g., a tumor cell. In one embodiment, Nectin-4-binding by the antibody is mediated by a single antigen binding domain or by two identical antigen binding domains, optionally wherein each antigen binding domain comprises a VH and VL domain pair or a single variable domain (e.g. sdAb).

When used in therapy for the elimination of Nectin-4-expressing tumor cells, either as an immunoconjugate or as non-conjugated (“naked”) antibody (e.g. optionally in combination with a cytotoxic agent administered separately), the Nectin-4 binding antibody (e.g. full-length antibody, antibody fragment) or a protein, conjugate or complex comprising such antibody may advantageously inhibit cell-cell interactions mediated by Nectin-4, e.g., as determined by assessing cell cluster formation (e.g., in a 3-dimensional cell culture system, by assessing tumor spheroid formation or growth) and/or anchorage-independent growth of Nectin-4 expressing cells. When used in therapy for the elimination of Nectin-4-expressing tumor cells, either as an immunoconjugate with a cytotoxic agent or as non-conjugated (“naked”) antibody in combination with a cytotoxic agent administered separately, the Nectin-4 binding antibody may advantageously be capable of sensitizing tumors to the cytotoxic agent, for example the antibody may enhance the ability of the cytotoxic agent to inhibit the proliferation or cause the death of tumor cells, for example to inhibit the proliferation or cause the death of tumor cells that are in clusters (e.g. in spheroids), or to inhibit the formation or growth of tumor cell clusters (e.g. spheroids). When used in therapy for the elimination of Nectin-4-expressing tumor cells as an immunoconjugate, the anti-Nectin-4 immunoconjugate may advantageously be capable of causing the death of Nectin-4-expressing tumor cells when conjugated to a cytotoxic molecule as disclosed herein.

Tumor cells that express Pgp can have decreased sensitivity to certain cytotoxic agents. Antibodies and cytotoxic agents (whether or not the cytotoxic agent is conjugated to the antibody) can optionally be tested using cells (e.g. tumor cells) that express Pgp.

Tumor spheroids are generally less sensitive to chemotherapy, in part due to the protection afforded by their structure, but also due to their slower proliferation rate, and consequently can be useful to assess the anti-tumor effect of the antibodies or immunoconjugates. Antibodies or immunoconjugates can be tested for the ability to inhibit spheroid formation in a concentration-dependent manner, or to disrupt cancer cells spheroid formation. Antibodies or immunoconjugates can for example be tested for their ability to alter spheroid formation in different cancer cell lines using real-time digital photography.

The antibodies or immunoconjugates can for example be characterized as being able to maintain (or increase maintenance of) the cancer cells as single cells, which cells may thereby be made more sensitive to a cytotoxic agent (e.g. a chemotherapy). Promoting maintenance of cancer cells as single cells to increase sensitivity to chemotherapy can be tested in vitro by adding different chemotherapeutic agents (e.g., doxorubicine, cisplatin, paclitaxel, camptothecin or a camptothecin analog) separately or in combination at different concentrations in the presence of antibodies or immunoconjugates, optionally further with comparison to antibodies or immunoconjugates that bind solely to the Ig-like V domain or the Ig-like C2-type 1 or 2 domains. Maintenance of cancer cells as single cells to increase sensitivity to chemotherapy can for example also be tested in vitro by adding different immunoconjugates targeting different domains of Nectin-4 with different toxins, where IC₅₀ of chemotherapies or ADCs will be lower where the antibody binds to the VC1 domain of Nectin-4, or the dose used to control tumor growth will be lower for the ADCs wherein the antibody bind the VC1 domain of Nectin-4. Sensitivity to chemotherapy can be assessed as cell viability, cell proliferation or cytotoxicity, for example monitored using different read-outs such as CTG by luminescence, confluence using Incucyte or caspase 3/7 or Annexin V. In one embodiment, an anti-Nectin-4 antibody or antibody fragment binds to a wild-type human Nectin-4 polypeptide, e.g. a polypeptide having the amino acid sequence of SEQ ID NO: 1, as well to a modified human Nectin-4 polypeptide having the amino acid sequence of SEQ ID NO: 32 (Nectin-4 protein containing the Ig-like C2 type 1 and 2 domains but lacking the Ig-like V type domain. Optionally, the antibody retains partial binding to the polypeptide lacking the Ig-like V type domain, optionally wherein the antibody retains binding to the Nectin-4 protein lacking the Ig-like V type domain at a reduced level compared to binding to a wild-type Nectin-4 protein, for example wherein the reduced level is between 5-50%, optionally 5-30%, optionally 5-25%, optionally 5-15% of binding to wild-type Nectin-4. Optionally further the anti-Nectin-4 antibody does not bind a Nectin-4 polypeptide lacking both the Ig-like V type and the Ig-like C2 type 1 domains, e.g., a polypeptide having the amino acid sequence of SEQ ID NO: 33. Optionally, binding is assessed by flow cytometry using cells made to express the Nectin-4 polypeptide and fluorescence intensity level (e.g. MFI) is determined.

In one embodiment, the anti-Nectin-4 antibody or antibody fragment does not bind to any of a human Nectin 1 protein, a human Nectin 2 protein, a human Nectin 3 protein and a human PVR protein. The respective Nectin or PVR protein can be specified as being expressed at the surface of a cell.

In one embodiment, an anti-Nectin-4 antibody or antibody fragment binds:
a Nectin-4 polypeptide having the amino acid sequence of SEQ ID NO: 1 and
a Nectin-4 polypeptide having the amino acid sequence of SEQ ID NO: 32 (optionally at a reduced level, optionally a level between 5-50% compared to binding to SEQ ID NO: 1), wherein the anti-Nectin-4 antibody or antibody fragment does not bind any of:
a polypeptide having the amino acid sequence of SEQ ID NO: 37,
a polypeptide having the amino acid sequence of SEQ ID NO: 38,
a polypeptide having the amino acid sequence of SEQ ID NO: 39, and
a polypeptide having the amino acid sequence of SEQ ID NO: 40.

Optionally further, the anti-Nectin-4 antibody or antibody fragment does not bind a polypeptide having the amino acid sequence of SEQ ID NO: 33.

Optionally further, the anti-Nectin-4 antibody or antibody fragment binds a rat Nectin-4 polypeptide (e.g. the amino acid sequence of SEQ ID NO: 35).

In one embodiment, an anti-Nectin-4 antibody or antibody fragment binds to the VC1 bridging domain, epitope or determinant of Nectin-4.

In one embodiment, an anti-Nectin-4 antibody or antibody fragment binds to human Nectin-4 at 1, 2, 3, 4 or 5 or more residues selected from the group consisting of A72, G73, S195, K197, S199, L150, S152, Q234 and 1236 (with reference to SEQ ID NO: 1). In one embodiment, an anti-Nectin-4 antigen binding protein or antibody binds to human Nectin-4 at 1, 2 or 3 residues selected from the group consisting of K197, S199 and Q234, optionally residues K197 and/or S199. In one embodiment, an anti-Nectin-4 antibody or antibody fragment binds to residue K197 of human Nectin-4. In one embodiment, an anti-Nectin-4 antibody or antibody fragment binds to residue S199 of human Nectin-4. In one embodiment, an anti-Nectin-4 antibody or antibody fragment binds to residue Q234 of human Nectin-4. Binding can be determined, for example, by assessing whether the antibody or antibody fragment has decreased or loss of binding to a mutant Nectin-4 polypeptide (e.g. as expressed at the surface of a cell) in which said residue is substituted by a different residue.

In one embodiment, an anti-Nectin-4 antibody or antibody fragment is capable of binding to both the Ig-like V type domain and the Ig-like C2 type 1 domain, for example to a determinant or epitope on the Ig-like V type domain and to a determinant or epitope on the Ig-like C2 type 1 domain.

In one embodiment, an anti-Nectin-4 antibody or antibody fragment retains at least partial binding to a modified Nectin-4 polypeptide comprising the Ig-like C2 type 1 domain but lacking the Ig-like V type domain, e.g. a Nectin-4 polypeptide lacking all or part of the amino acid sequence of SEQ ID NO: 3. Partial binding can be characterized as retaining binding, but at a reduced level (e.g. as assessed by flow cytometry) compared to binding to a wild-type human Nectin-4 polypeptide having the amino acid sequence of residue of SEQ ID NO: 1. The Nectin-4 polypeptides can be specified as being expressed at the surface of a cell (e.g., a host cell made to express the polypeptide(s).

Antibodies may be produced by a variety of techniques known in the art. Typically, they are produced by immunization of a non-human animal, preferably a mouse, with an immunogen comprising a Nectin-4 polypeptide, preferably a human Nectin-4 polypeptide. The Nectin-4 polypeptide may comprise the full length sequence of a human Nectin-4 polypeptide, or a fragment or derivative thereof, typically an immunogenic fragment, i.e., a portion of the polypeptide comprising an epitope exposed on the surface of cells expressing a Nectin-4 polypeptide, for example the epitope recognized by the 5E7, 10B12, 6C11B or 6A7 antibody. The Nectin-4 polypeptide can be specified as comprising or consisting of VC1 bridging domain or a fragment or subsequence thereof. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human Nectin-4 polypeptide in a lipid membrane, typically at the surface of a cell. In a specific embodiment, the immunogen comprises intact cells, particularly intact human cells, optionally treated or lysed. In another preferred embodiment, the polypeptide is a recombinant Nectin-4 polypeptide. In a specific embodiment, the immunogen comprises intact Nectin-4-expressing cells.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, NY (1988), the entire disclosure of which is herein incorporated by reference).

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that compete for binding to Nectin-4, with monoclonal antibody 5E7, 10B12, 6C11B or 6A7 can be readily determined using any one of a variety of immunological screening assays in which antibody competition can be assessed. Many such assays are routinely practiced and are well known in the art (see, e.g., U.S. Pat. No. 5,660,827, which is incorporated herein by reference).

For example, where the test antibodies to be examined are obtained from different source animals, or are even of a different Ig isotype, a simple competition assay may be employed in which the control (5E7, 10B12, 6C11B or 6A7, for example) and test antibodies are admixed (or pre-adsorbed) and applied to a sample containing Nectin-4 polypeptides. Protocols based upon western blotting and the use of Surface Plasmon Resonance (e.g. Biacore™) analysis are suitable for use in such competition studies.

In certain embodiments, one pre-mixes the control antibodies (5E7, 10B12, 6C11B or 6A7, for example) with varying amounts of the test antibodies (e.g., about 1:10 or about 1:100) for a period of time prior to applying to the Nectin-4 antigen sample. In other embodiments, the control and varying amounts of test antibodies can simply be admixed during exposure to the Nectin-4 antigen sample. As long as one can distinguish bound from free antibodies (e.g., by using separation or washing techniques to eliminate unbound antibodies) 5E7, 10B12, 6C11B or 6A7 from the test antibodies (e.g., by using species-specific or isotype-specific secondary antibodies or by specifically labeling 5E7, 10B12, 6C11B or 6A7 with a detectable label) one can determine if the test antibodies reduce the binding of 5E7, 10B12, 6C11B or 6A7 to the antigens. The binding of the (labeled) control antibodies in the absence of a completely irrelevant antibody can serve as the control high value. The control low value can be obtained by incubating the labeled (5E7, 10B12, 6C11B or 6A7) antibodies with unlabelled antibodies of exactly the same type (5E7, 10B12, 6C11B or 6A7), where competition would occur and reduce binding of the labeled antibodies. A test antibody may for example reduce the binding of 5E7, 10B12, 6C11B or 6A7 to Nectin-4 antigens by at least about 50%, such as at least about 60%, or more preferably at least about 80% or 90% (e.g., about 65-100%), at any ratio of 5E7, 10B12, 6C11B or 6A7: test antibody between about 1:10 and about 1:100. Optionally, such test antibody will reduce the binding of 5E7, 10B12, 6C11B or 6A7 to the Nectin-4 antigen by at least about 90% (e.g., about 95%).

Competition can also be assessed by, for example, a flow cytometry test. In such a test, cells bearing a given Nectin-4 polypeptide can be incubated first with 5E7, 10B12, 6C11B or 6A7, for example, and then with the test antibody labeled with a fluorochrome or biotin. The antibody is said to compete with 5E7, 10B12, 6C11B or 6A7 if the binding obtained upon preincubation with a saturating amount of 5E7, 10B12, 6C11B or 6A7 is about 80%, preferably about 50%, about 40% or less (e.g., about 30%, 20% or 10%) of the binding (as measured by mean of fluorescence) obtained by the antibody without preincubation with 5E7, 10B12, 6C11B or 6A7. Alternatively, an antibody is said to compete with 5E7, 10B12, 6C11B or 6A7 if the binding obtained with a labeled 5E7, 10B12, 6C11B or 6A7 antibody (by a fluorochrome or biotin) on cells preincubated with a saturating amount of test antibody is about 80%, preferably about 50%, about 40%, or less (e.g., about 30%, 20% or 10%) of the binding obtained without preincubation with the test antibody.

A simple competition assay in which a test antibody is pre-adsorbed and applied at saturating concentration to a surface onto which a Nectin-4 antigen is immobilized may also be employed. The surface in the simple competition assay is preferably a Biacore™ chip (or other media suitable for surface plasmon resonance analysis). The control antibody (e.g., 5E7, 10B12, 6C11B or 6A7) is then brought into contact with the surface at a Nectin-4-saturating concentration and the Nectin-4 and surface binding of the control antibody is measured. This binding of the control antibody is compared with the binding of the control antibody to the Nectin-4-containing surface in the absence of test antibody. In a test assay, a significant reduction in binding of the Nectin-4-containing surface by the control antibody in the presence of a test antibody indicates that the test antibody recognizes substantially the same region on Nectin-4 as the control antibody such that the test antibody “cross-reacts” with the control antibody. A test antibody may for example reduce the binding of control (such as 5E7, 10B12, 6C11B or 6A7) antibody to a Nectin-4 antigen by at least about 30% or more, preferably about 40%. Optionally, such a test antibody will reduce the binding of the control antibody (e.g., 5E7, 10B12, 6C11B or 6A7) to the Nectin-4 antigen by at least about 50% (e.g., at least about 60%, at least about 70%, or more). It will be appreciated that the order of control and test antibodies can be reversed: that is, the control antibody can be first bound to the surface and the test antibody is brought into contact with the surface thereafter in a competition assay. Preferably, the antibody having higher affinity for the Nectin-4 antigen is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183:33-41, the disclosure of which is incorporated herein by reference.

The antibodies will bind to Nectin-4-expressing tumor cells from an individual or individuals with a cancer characterized by Nectin-4-positive tumor cells, i.e., an individual that is a candidate for treatment with one of the herein-described methods using an anti-Nectin-4 antibody. Accordingly, once an antibody that specifically recognizes Nectin-4 on cells is obtained, it can optionally be tested for its ability to bind to Nectin-4-positive cells (e.g., cancer cells). It can optionally be tested for its ability to bind to tumor cells that express at their surface high and/or tumor cells that express at their surface low levels of Nectin-4 polypeptides. In particular, prior to treating a patient with one of the present antibodies, one may optionally test the ability of the antibody to bind malignant cells taken from the patient, e.g., in a blood sample or tumor biopsy, to maximize the likelihood that the therapy will be beneficial in the patient.

In one embodiment, the antibodies are validated in an immunoassay to test their ability to bind to Nectin-4-expressing cells, e.g., malignant cells. For example, a blood sample or tumor biopsy is performed and tumor cells are collected. The ability of a given antibody to bind to the cells is then assessed using standard methods well known to those in the art. To assess the binding of the antibodies to the cells, the antibodies can either be directly or indirectly labeled. When indirectly labeled, a secondary, labeled antibody is typically added.

Determination of whether an antibody binds within an epitope region can be carried out in ways known to the person skilled in the art. As one example of such mapping/characterization methods, an epitope region for an anti-Nectin-4 antibody may be determined by epitope “foot-printing” using chemical modification of the exposed amines/carboxyls in the Nectin-4 protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) Engen, J. R. and Smith, D. L. (2001) Anal. Chem. 73, 256fA-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang et al., Journal of Molecular Biology, Vol. 281 (1) pp. 61-67 (1998); and Saito and Patterson, Methods. 1996 June; 9 (3): 516-24.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801. Protease digestion techniques also can be useful in the context of epitope mapping and identification. Antigenic determinant-relevant regions/sequences can be determined by protease digestion, e.g., by using trypsin in a ratio of about 1:50 to Nectin-4 or o/n digestion at and pH 7-8, followed by mass spectrometry (MS) analysis for peptide identification. The peptides protected from trypsin cleavage by the anti-Nectin-4 binder can subsequently be identified by comparison of samples subjected to trypsin digestion and samples incubated with antibody and then subjected to digestion by e.g., trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the Nectin-4 polypeptide that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is re-placed with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron micros-copy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE™) and reflectometric interference spectroscopy (RifS). See, e.g., Fagerstam et al., Journal Of Molecular Recognition 1990; 3:208-14; Nice et al., J. Chromatogr. 1993; 646:159-168; Leipert et al., Angew. Chem. Int. Ed. 1998; 37:3308-3311; Kröger et al., Biosensors and Bioelectronics 2002; 17:937-944.

It should also be noted that an antibody binding the same or substantially the same epitope as an antibody can be identified in one or more of the exemplary competition assays described herein.

Upon immunization and production of antibodies in a vertebrate or cell, or upon generation of a library of candidate antibodies or antibody sequences, particular selection steps may be performed to isolate antibodies as claimed. In this regard, in a specific embodiment, provided are methods of producing such antibodies, comprising: (a) immunizing a non-human mammal with an immunogen comprising a Nectin-4 polypeptide or preparing a library of antibodies or antibody sequences; and (b) preparing antibodies from said immunized animal, or from said library of antibodies or sequences; and (c) selecting antibodies from step (b) that are capable of binding Nectin-4, optionally selecting antibodies from step (b) that are capable of binding to the VC1 bridging domain of Nectin-4. Optionally, selecting antibodies from step (b) that are capable of binding to the VC1 bridging domain of Nectin-4 can be carried out by any suitable method, including but not limited to those described herein (e.g., epitope mapping/characterization methods, testing whether antibodies having reduced binding to Nectin-4 mutant(s), testing whether antibodies bind to Nectin-4 proteins modified to lack the Ig-like V domain, etc.

In one aspect an antibody can have an average disassociation constant (K_{D}) of no more than 1×10⁻⁸ M, optionally less than 1×10⁻⁹ M with respect to human Nectin-4, as determined by, e.g., surface plasmon resonance (SPR) screening (such as by analysis with a BIAcore™ SPR analytical device). In a more particular exemplary aspect, provided are anti-Nectin-4 antibodies that have a K_{D} of about 1×10⁻⁸ M to about 1×10⁻¹⁰ M, or about 1×10⁻⁹ M to about 1×10⁻¹¹ M, for Nectin-4.

In one embodiment, the antibodies of the disclosure that bind Nectin-4 are capable of inhibiting cell-cell interactions mediated by Nectin-4, e.g., as determined by assessing cell cluster formation and/or anchorage-independent growth of Nectin-4 expressing cells. For example, one can bring Nectin-4 expressing tumor cells into contact with an antibody that binds Nectin-4, and assessing whether the antibody reduces cell-cell adhesion between Nectin-4 expressing tumor cells, and/or reducing growth and/or cluster formation of Nectin-4 expressing tumor cells (as assessed, for example, using 3-dimensional or non-adherent tumor cell culture; tumor spheroid assays).

In one embodiment, the antibodies of the disclosure that bind Nectin-4 are capable of inhibiting cell-cell interactions mediated by Nectin-4, as determined by assessing spheroid formation by the Nectin-4 expressing tumor cells. Determining whether an antibody is capable of inhibiting spheroid formation can for example comprise culturing Nectin-4 expressing tumor cells in 3-dimensional or non-adherent tumor cell culture (e.g. in a tumor spheroid assays) in the presence of an antibody that binds Nectin-4, and assessing whether the antibody inhibits spheroid formation.

In one embodiment, provided is a method for identifying or selecting an antibody for use in the preparation or production of an immunoconjugate (e.g. antibody drug conjugate), or for use in the treatment of a cancer (e.g. in combination with a chemotherapeutic agent, or as an ADC), the method comprising: bringing an antibody that binds Nectin-4 into contact with a Nectin-4 expressing tumor cell (e.g. in 3-dimensional or non-adherent tumor cell culture; in a tumor spheroid assay), and assessing whether the antibody inhibits cluster formation (e.g. spheroid formation) and/or anchorage-independent growth of the Nectin-4 expressing tumor cells]. Optionally, the method comprises selecting an antibody for use in treatment of a cancer upon a determination that the antibody is capable of inhibiting cluster formation and/or anchorage-independent growth of the Nectin-4 expressing tumor cells.

In one embodiment, provided is a method for identifying or selecting an antibody for use in the preparation or production of an immunoconjugate (e.g. antibody drug conjugate), or for use in the treatment of a cancer (e.g. in combination with a chemotherapeutic agent, or as an ADC), the method comprising: bringing an antibody that binds Nectin-4 into contact with a Nectin-4 expressing tumor cell (e.g., a cell in 3-dimensional or non-adherent tumor cell culture; tumor spheroid assays) and assessing whether the antibody inhibits spheroid formation by the Nectin-4 expressing tumor cells. Optionally, the method comprises selecting an antibody for use in treatment of a cancer upon a determination that the antibody is capable of inhibiting spheroid formation by the Nectin-4 expressing tumor cells.

Optionally in any of the methods, the tumor cells express Pgp (MDR1).

Optionally, in any of the methods, the method further comprises a step of conjugating the antibody, either before or after assessment of inhibition of tumor or tumor cell growth, to a cytotoxic agent (e.g., a camptothecin analogue), preferably via a linker moiety. Optionally an immunoconjugate of the description is thereby produced.

In one embodiment, selecting an antibody for use in preparation or production of an immunoconjugate (e.g. antibody drug conjugate), or for use in the treatment of a cancer (e.g. a Nectin-4 positive cancer), can comprise contacting cells (e.g. tumor cells) that express at their surface Nectin-4 polypeptides with an antibody drug conjugate that binds Nectin-4, and assessing the ability (e.g. the potency, the IC₅₀) of the antibody drug conjugate to cause the death of the cells. In one embodiment, an antibody that is selected is more potent (e.g. a lower IC₅₀) in causing death of the cells compared to an antibody drug conjugate having the VH and VL of antibody enfortumab (when the respective ADC have the same linker-cytotoxic drug and drug:antibody ratio). Optionally, the antibody drug conjugate comprises an antibody that binds Nectin-4 conjugated to a camptothecin analogue.

In one embodiment, provided are methods that can be used to select and/or identify antibodies suitable for use in the methods herein. The methods can also be advantageously used as “bioassays” during the manufacturing process of an antibody drug conjugate in order to test and/or monitor the quality or activity of the antibody drug conjugate. In one example, the methods may comprise for example the steps of: (i) providing an antibody that inhibits the cluster formation and/or anchorage-independent growth of the Nectin-4 expressing tumor cells, wherein the antibody is conjugated to a cytotoxic agent, and (ii) contacting cells that express at their surface Nectin-4 polypeptides with the antibody drug conjugate of step (i), and assessing the ability (e.g. the potency, the IC₅₀) of the antibody drug conjugate to cause the death of the cells. Optionally, the antibody drug conjugate is selected (e.g., for further processing in manufacturing, for filling into a container such as a vials or syringes, for use in treatment of cancer, etc.) if the antibody drug conjugate is determined to be able to cause the death of the cells, optionally with a predetermined efficacy or potency (e.g., as compared to a reference standard antibody drug conjugate composition, as compared to a predetermined IC₅₀ value).

“IC₅₀” (or EC₅₀) with respect to binding to an activity assay (e.g. ability to cause the death of the cells), refers to the inhibitory or efficient concentration of anti-Nectin-4 antibody which produces 50% of its maximum response or effect with respect to the activity of interest.

Assays herein can be particular useful for antibodies that bind the VC1 bridging domain. For example, in one embodiment, provided is a method of making, identifying or testing a Nectin-4-binding antibody or antibody drug conjugate comprising such Nectin-4-binding antibody, the method comprising bringing an antibody or antibody drug conjugate (or sample from a production batch thereof) that displays reduced binding to a mutant Nectin-4 polypeptide comprising a mutation at residues K197 and/or S199, relative to binding between the antibody and a wild-type Nectin-4 polypeptide, into contact with a cell expressing at its surface Nectin-4, optionally a wild-type Nectin-4 polypeptide, and assessing the ability of the antibody or antibody drug conjugate to undergo intracellular internalization, induces intracellular internalization of the antibody-Nectin-4 complex, and/or cause death of the cell.

In some embodiments, provided is a method of testing an antibody that binds the VC1 bridging domain of Nectin-4, the method comprising: (i) bringing the antibody into contact with a cell expressing at its surface Nectin-4 (optionally wherein the cell expresses Pgp) in the presence of cytotoxic agent, optionally wherein the antibody is conjugated to the cytotoxic agent, and (ii) assessing the ability of the antibody or conjugated antibody to cause death of the cell, optionally assessing whether the antibody or conjugated antibody in the presence of cytotoxic agent increases cell death compared to that observed in presence of the cytotoxic agent alone. In one embodiment, the cell expresses Pgp and the cytotoxic agent is a camptothecin analogue, optionally exatecan.

In some embodiments, provided is a method of testing an ADC comprising an antibody that binds the VC1 bridging domain of Nectin-4 conjugated to a cytotoxic agent (e.g. a camptothecin analogue, an exatecan), the method comprising: (i) bringing the ADC into contact with a cell expressing at its surface Nectin-4 (optionally wherein the cell expresses Pgp) in the presence of cytotoxic agent, and (ii) assessing the ability of the ADC to cause death of the cell, optionally compared to a reference ADC, optionally compared to cell death observed in presence of the cytotoxic agent alone.

In one aspect of any of the embodiments, the antibodies prepared according to the present methods are monoclonal antibodies. In another aspect, the non-human animal used to produce antibodies is a mammal, such as a rodent, bovine, porcine, fowl, horse, rabbit, goat, or sheep. Antibodies of the invention can optionally be specified to be antibodies other than any of antibodies 5E7, 10B12, 6C11B or 6A7, or derivatives thereof, e.g., that comprise their respective heavy and light chain CDRs or the antigen binding region in whole or in part.

DNA encoding an antibody that binds an epitope present on Nectin-4 polypeptides is isolated from a hybridoma and placed into an expression vector(s), which is then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. In one embodiment, provided is an isolated nucleic acid sequence encoding a light chain and/or a heavy chain of an antibody, as well as a recombinant host cell comprising (e.g., in its genome) such nucleic acid.

In one aspect, the anti-Nectin-4 antibody is an antibody that is a function-conservative variant of an antibody having the heavy and light chain CDRs or variable regions of antibody 5E7, 10B12, 6C11B or 6A7. “Function-conservative variants” are those in which a given amino acid residue in a protein (e.g., an antibody or antibody fragment) has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native, reference or parent protein to which it is compared.

Antibodies can be assessed and/or selected based on binding to the VC1 bridging domain of a Nectin-4 polypeptide, e.g. at the cell surface as a membrane-anchored Nectin-4 polypeptide lacking the Ig-like V domain. In one aspect, the antibodies bind an antigenic determinant present on the Ig-like V domain and an antigen determined present on the C2 type 1 domain of a Nectin-4 polypeptide, e.g. as present in a protein expressed at the cell surface. In one embodiment, the determinant is not present entirely on the Ig-like V domain and not present entirely on the Ig-like C2 type 1 domain. In one embodiment, the antibody has partial reduction in binding to a membrane-anchored Nectin-4 polypeptide lacking the Ig-like V domain (i.e. a Nectin-4 polypeptide having the amino acid sequence shown in SEQ ID NO: 32), compared to a wild-type membrane-bound Nectin-4 polypeptide.

In one aspect, provided is a method of testing and/or producing an anti-Nectin-4 antibody (e.g. an antibody suitable for use in an immunoconjugate with a cytotoxic agent), comprising:
(a) providing a plurality of antibodies that bind a Nectin-4 protein,
(b) assessing whether the antibodies bind to the antibodies to the VC1 bridging domain of Nectin-4, and
(c) optionally, selecting antibodies (e.g., from those assessed in step (b)) that bind to the antibodies to the VC1 bridging domain of Nectin-4. Optionally, step (b) may comprise the step of assessing whether the antibody has partial reduction in binding to a membrane-anchored Nectin-4 polypeptide lacking the Ig-like V domain, and/or whether the antibody has reduced binding to a mutant Nectin-4 polypeptide comprising one or more amino acid substitutions (e.g., at residues A72, G73, S195, K197, S199, L150, S152, Q234 and 1236), optionally a mutant Nectin-4 polypeptide comprising one or more amino acid substitutions at residues K197, S199 and/or Q234; optionally a mutant Nectin-4 polypeptide comprising the substitutions S195A/K197T/S199A, optionally a mutant Nectin-4 polypeptide comprising the substitutions A72P/G73N/K197T/S199A, or optionally a mutant Nectin-4 polypeptide comprising the substitutions the substitutions L150S/S152A/Q234R/1236S.

In one aspect, provided is a method of testing and/or producing an anti-Nectin-4 antibody (e.g. an antibody suitable for use in an immunoconjugate with a cytotoxic agent, optionally a camptothecin analogue), comprising:
(a) providing a plurality of antibodies that bind a Nectin-4 protein,
(b) assessing whether the antibodies cause inhibition of cluster formation and/or anchorage-independent growth of Nectin-4 expressing tumor cells, and
(c) optionally, selecting antibodies (e.g., from those assessed in step (b)) that cause inhibition of cluster formation and/or anchorage-independent growth of Nectin-4 expressing tumor cells. Optionally, step (b) may additionally comprise the step of assessing whether antibodies bind and/or selecting antibodies that bind to the antibodies to the VC1 bridging domain of Nectin-4.

In one aspect, provided is a method of testing and/or producing an anti-Nectin-4 antibody (e.g. an antibody suitable for use in an immunoconjugate with a cytotoxic agent, optionally a camptothecin analogue), comprising:
(a) providing a plurality of antibodies that bind a Nectin-4 protein,
(b) assessing whether the antibodies are capable of reducing cell-cell adhesion between Nectin-4 expressing tumor cells, and/or reducing growth and/or cluster formation of Nectin-4 expressing tumor cells (as assessed, for example, using 3-dimensional or non-adherent tumor cell culture; tumor spheroid assays), and
(c) optionally, selecting antibodies (e.g., from those assessed in step (b)) that are capable of reducing cell-cell adhesion between Nectin-4 expressing tumor cells, and/or reducing growth and/or cluster formation of Nectin-4 expressing tumor cells. Optionally, step (b) may additionally comprise the step of assessing whether antibodies bind and/or selecting antibodies that bind to the antibodies to the VC1 bridging domain of Nectin-4.

In one aspect, provided is a method of testing and/or producing an anti-Nectin-4 antibody (e.g. an antibody suitable for use in an immunoconjugate with a cytotoxic agent, optionally a camptothecin analogue), comprising:
(a) providing a plurality of antibodies that bind a Nectin-4 protein,
(b) assessing whether the antibodies are capable of inhibiting cell-cell interactions mediated by Nectin-4, e.g., as determined by assessing cell cluster formation and/or anchorage-independent growth of Nectin-4 expressing cells, and
(c) optionally, selecting antibodies (e.g., from those assessed in step (b)) that capable of inhibiting cell-cell interactions mediated by Nectin-4.

In one aspect, provided is a method of testing and/or producing an anti-Nectin-4 antibody (e.g. an antibody suitable for use in an immunoconjugate with a cytotoxic agent, optionally a camptothecin analogue), comprising:
(a) providing one or a plurality of antibodies that bind a Nectin-4 protein,
(b) assessing whether the antibody is capable sensitizing a tumor (e.g. metastatic cancer, Nectin-4 expressing tumor) to a cytotoxic agent (e.g. an agent (Z) disclosed herein, a camptothecin analogue), and
(c) optionally, selecting an antibody (e.g., from in step (b)) capable of sensitizing a tumor to a cytotoxic agent (e.g. an agent (Z) disclosed herein, a camptothecin analogue). Optionally, step (b) may additionally comprise bringing each antibody into contact with tumor cells, optionally Pgp-expressing tumor cells (e.g. in 3-dimensional tumor cell culture, in a tumor spheroid assay) in the presence of a cytotoxic agent, and determining whether the antibody enhances the ability of the cytotoxic agent to cause the death of the tumor cells compared to the cytotoxic agent in the absence of antibody. Optionally, step (b) may additionally comprise the step of assessing whether antibody binds and/or selecting an antibody that bind to the antibodies to the VC1 bridging domain of Nectin-4.

In one aspect, provided is a method of testing and/or producing an ADC (e.g. an ADC comprising an anti-Nectin-4 antibody that binds the VC1 bridging domain conjugated to a topoisomerase I inhibitor, a camptothecin analogue, an exatecan), the method comprising:
(a) providing an ADC comprising an anti-Nectin-4 antibody that binds the VC1 bridging domain conjugated to a topoisomerase I inhibitor, optionally a camptothecin analogue, optionally an exatecan),
(b) bringing the ADC into contact with Pgp-expressing tumor cells and assessing whether the antibody is capable of causing the death of the Pgp-expressing tumor cells (e.g. comprising determining the potency of, the IC₅₀ for, causing cell death), and
(c) optionally, selecting the ADC if is capable of causing the death of the Pgp-expressing tumor cells. Optionally, step (b) may comprise determining whether the ADC is capable causing the death of the Pgp-expressing tumor cells compared to the topoisomerase I inhibitor alone (in the absence of antibody). Optionally, step (c) may comprise selecting the ADC if is capable of causing increased death of the Pgp-expressing tumor cells compared to the topoisomerase I inhibitor alone (in the absence of antibody).

In one aspect, provided is a method of selecting or testing an immunoconjugate, or testing the suitability of a topoisomerase inhibitor agent for use in an anti-Nectin-4 immunoconjugate (e.g. for use in combination with the anti-Nectin-4 antibody, for use in an immunoconjugate by conjugation to the anti-Nectin-4 antibody), comprising:
(a) providing an anti-Nectin-4 antibody, optionally wherein antibody binds to the VC1 bridging domain of Nectin-4,
(b) providing a topoisomerase inhibitor agent (e.g. one or a plurality of agents to be assessed separately in step c) and conjugating the agent (e.g. conjugating separately each agent if a plurality are to be assessed) to the antibody of (a),
(c) assessing whether the topoisomerase inhibitor-conjugated antibody of step (b) is capable causing the death of a Pgp-expressing tumor cell, and optionally, selecting an immunoconjugate or a topoisomerase inhibitor for use in an immunoconjugate if the ability to cause cell death is increased by topoisomerase inhibitor-conjugated antibody, optionally compared to an immunoconjugate comprising a reference or comparator topoisomerase inhibitor-conjugated antibody. The comparator or reference can comprise a different topoisomerase inhibitor and/or a different anti-Nectin-4 antibody.

In one aspect, provided is a method of selecting or testing the suitability of a chemotherapeutic or cytotoxic agent for use with an anti-Nectin-4 antibody that binds to the VC1 bridging domain of Nectin-4 (e.g. for use in combination with the anti-Nectin-4 antibody, for use in an immunoconjugate by conjugation to the anti-Nectin-4 antibody), comprising: (a) providing an anti-Nectin-4 antibody that binds to the VC1 bridging domain of Nectin-4,
(b) providing a cytotoxic or chemotherapeutic agent (e.g. one or a plurality of agents to be assessed separately in step c),
(c) assessing whether the antibody is capable sensitizing a tumor (e.g. a Pgp-expressing tumor, a tumor from a metastatic cancer, Nectin-4 expressing tumor) to the agent, and
(d) optionally, selecting an agent whose anti-tumor activity is increased by the antibody (e.g. for use in combination with the anti-Nectin-4 antibody, for use in an immunoconjugate by conjugation to the anti-Nectin-4 antibody). Optionally, step € may additionally comprise bringing the antibody into contact with tumor cells (e.g. Pgp-expressing cells, cells in 3-dimensional tumor cell culture, in a tumor spheroid assay) in the presence of the agent, and determining whether the antibody enhances the ability of the cytotoxic agent to cause the death of the tumor cells compared to the cytotoxic agent in the absence of antibody. Optionally, step (a) may additionally comprise the step of assessing whether antibody binds and/or selecting an antibody that bind to the antibodies to the VC1 bridging domain of Nectin-4.

In any of the above methods of producing an antibody, the method may further comprise the step of assessing the binding affinity of an antibody to a human Nectin-4 polypeptide, for example assessing 1:1 Binding fit and/or dissociation or off rate (kd (1/s)), as determined in a SPR monovalent binding affinity assay.

The antibody tested, selected and/or produced can then be used for further evaluation, for further processing, production of a quantity of, for conjugation to a cytotoxic agent, for formulation or filling into a container or vial, for use in treatment of cancer. In one aspect, provided is an antibody or antibody drug conjugated produced by the methods.

In one example, antibodies screening or testing can comprise use of mutant Nectin-4 polypeptides to characterize and/or orient the selection of antibodies. For example, a method of producing or testing an antibody which binds Nectin-4 can comprise the steps of:
(a) providing a plurality of antibodies that bind a Nectin-4 polypeptide,
(b) bringing each of said antibodies into contact with a mutant Nectin-4 polypeptide comprising a mutation at 1, 2, 3, 4 or 5 or more residues selected from the group consisting of A72, G73, S195, K197, S199, L150, S152, Q234 and 1236, or optionally at residues K197 and/or S199, or optionally at 1, 2 or 3 residues selected from the group consisting of K197, S199 and Q234 (with reference to SEQ ID NO: 1), and assessing binding between the antibody and the mutant Nectin-4 polypeptide, relative to binding between the antibody and a wild-type Nectin-4 polypeptide comprising the amino acid sequence of SEQ ID NO: 1, and
€ optionally, selecting an antibody (e.g. for further evaluation, for conjugation to a cytotoxic agent, for further processing, production of a quantity of, for use in treatment) that has reduced binding to the mutant Nectin-4 polypeptide, relative to binding between the antibody and a wild-type Nectin-4 polypeptide comprising the amino acid sequence of SEQ ID NO: 1. Optionally the mutant Nectin-4 polypeptide comprises the substitutions S195A/K197T/S199A, the substitutions A72P/G73N/K197T/S199A, and/or the substitutions L150S/S152A/Q234R/1236S.

Advantageously, antibodies can optionally be identified and selected based on binding to the same region or epitope on the surface of the Nectin-4 polypeptide as any of the antibodies described herein. In one aspect, the antibodies bind substantially the same epitope as any of antibodies 5E7, 10B12, 6C11B or 6A7. In one embodiment, the antibodies bind to an epitope of Nectin-4 that at least partially overlaps with, or includes at least one residue in, the epitope bound by antibody 5E7, 10B12, 6C11B or 6A7. The residues bound by the antibody can be specified as being present on the surface of the Nectin-4 polypeptide, e.g., on n Nectin-4 polypeptide expressed on the surface of a cell.

Binding of anti-Nectin-4 antibody to a particular site on Nectin-4 can be assessed by measuring binding of an anti-Nectin-4 antibody to cells transfected with Nectin-4 mutants, as compared to the ability of anti-Nectin-4 antibody to bind wild-type Nectin-4 polypeptide (e.g., SEQ ID NO: 1). The disclosure provides mutant Nectin-4 polypeptides that permit characterization of the binding site of antibodies, including antibodies that bind to the VC domain. In one embodiment, provided is a mutant Nectin-4 polypeptide comprising the mutations indicated in the mutants of Table 2, nucleic acids encoding such mutants, and recombinant host cells expressing the foregoing. In one embodiment, provided is a mutant Nectin-4 polypeptide comprising a mutation at 1, 2, 3, 4 or 5 or more residues selected from the group consisting of A72, G73, S195, K197, S199, L150, S152, Q234 and 1236, or optionally at residues K197 and/or S199, or optionally at 1, 2 or 3 residues selected from the group consisting of K197, S199 and Q234 (with reference to SEQ ID NO: 1), nucleic acids encoding such mutants, and recombinant host cells expressing the foregoing. A reduction in binding between an anti-Nectin-4 antibody and a mutant Nectin-4 polypeptide (e.g., a mutant of Table 2) means that there is a reduction in binding affinity (e.g., as measured by known methods such FACS testing of cells expressing a particular mutant, or by Biacore testing of binding to mutant polypeptides) and/or a reduction in the total binding capacity of the anti-Nectin-4 antibody (e.g., as evidenced by a decrease in Bmax in a plot of anti-Nectin-4 antibody concentration versus polypeptide concentration). A significant reduction in binding indicates that the mutated residue is directly involved in binding to the anti-Nectin-4 antibody or is in close proximity to the binding protein when the anti-Nectin-4 antibody is bound to Nectin-4.

In some embodiments, a significant reduction in binding means that the binding affinity and/or capacity between an anti-Nectin-4 antibody and a mutant Nectin-4 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type Nectin-4 polypeptide. In certain embodiments, binding is reduced below detectable limits. In some embodiments, a significant reduction in binding is evidenced when binding of an anti-Nectin-4 antibody to a mutant Nectin-4 polypeptide is less than 50% (e.g., less than 45%, 40%, 35%, 30%, 25%, 20%, 15% or 10%) of the binding observed between the anti-Nectin-4 antibody and a wild-type Nectin-4 polypeptide.

In some embodiments, anti-Nectin-4 antibodies are provided that exhibit significantly lower binding for a mutant Nectin-4 polypeptide (e.g. a mutant of Table 2) in which a residue in a segment comprising an amino acid residue bound by antibody 5E7, 10B12, 6C11B or 6A7 is substituted with a different amino acid, compared to a binding to a wild-type Nectin-4 polypeptide not comprising such substitution(s) (e.g. a polypeptide of SEQ ID NO: 1).

In one aspect, an anti-Nectin-4 antibody binds an epitope positioned on or within the VC1 bridging domain of Nectin-4. In one aspect, an anti-Nectin-4 antibody competes with antibody 5E7, 10B12, 6C11B or 6A7 for binding to an epitope on the VC1 bridging domain of Nectin-4.

In one aspect, the anti-Nectin-4 antibody has reduced binding, optionally loss of binding, to an Nectin-4 polypeptide having a mutation at one, two or three residues selected from the group consisting of: K197 (e.g. a K197T substitution), S199 (e.g. a S199A substitution) and Q234 (e.g. a Q234R substitution), with reference to SEQ ID NO: 1.

In one embodiment, an antibody furthermore has reduced binding to a mutant Nectin-4 polypeptide comprising a mutation at one or more (or all of) residues selected from the group consisting of S195, K197 and S199 (with reference to SEQ ID NO: 1), optionally, the mutant Nectin-4 polypeptide has the mutations: S195A, K197T and S199A.

In one embodiment, an antibody furthermore has reduced binding to a mutant Nectin-4 polypeptide comprising a mutation at one or more (or all of) residues selected from the group consisting of A72, G73, K197 and S199 (with reference to SEQ ID NO: 1), optionally, the mutant Nectin-4 polypeptide has the mutations: A72P, G73N, K197T and S199A.

In one embodiment, an antibody furthermore has reduced binding to a mutant Nectin-4 polypeptide comprising a mutation at one or more (or all of) residues selected from the group consisting of L150, S152, Q234 and 1236 (with reference to SEQ ID NO: 1), optionally, the mutant Nectin-4 polypeptide has the mutations: L150S, S152A, Q234R and 1236S.

In one embodiment, an antibody furthermore has reduced binding to a mutant Nectin-4 polypeptide comprising a mutation at one or more (or all of) residues selected from the group consisting of A72, G73, S195, K197, S199, L150, S152, Q234 and 1236 (with reference to SEQ ID NO: 1), optionally, the mutant Nectin-4 polypeptide has the mutations: A72P, G73N, S195A, K197T, S199A, L150S, S152A, Q234R and 1236S.

In each case, a decrease or loss of binding can be specified as being relative to binding between the antibody and a wild-type Nectin-4 polypeptide comprising the amino acid sequence of SEQ ID NO: 1.

In one aspect, the anti-Nectin-4 antibody binds an epitope on Nectin-4 comprising an amino acid residue (e.g., one, two, three, four or five of the residues) selected from the group consisting of A72, G73, S195, K197, S199, L150, S152, Q234 and 1236, or optionally at 1, 2 or 3 residues selected from the group consisting of K197, S199 and Q234 (with reference to SEQ ID NO: 1).

In one aspect, the anti-Nectin-4 antibody binds an epitope on Nectin-4 comprising an amino acid residue (e.g., one, two or three of the residues) selected from the group consisting of S195, K197 and S199 (with reference to SEQ ID NO: 1).

In one aspect, the anti-Nectin-4 antibody binds an epitope on Nectin-4 comprising an amino acid residue (e.g., one, two, three or four of the residues) selected from the group consisting of A72, G73, K197 and S199.

In one aspect, the anti-Nectin-4 antibody binds an epitope on Nectin-4 comprising an amino acid residue (e.g., one, two, three or four of the residues) selected from the group consisting of L150, S152, Q234 and 1236.

In one aspect, an anti-Nectin-4 antibody binds an epitope positioned on or within the VC1 bridging domain of Nectin-4. In one aspect, an anti-Nectin-4 antibody competes with antibody 5E7, 10B12, 6C11B or 6A7 for binding to an epitope on the VC1 bridging domain of the human Nectin-4 protein.

In one embodiment, a VH region of an antibody of the disclosure comprises an amino acid sequence having at least about 80%, 85%, 90%, 95%, 97%, 98% or 99% identity, to the amino acid sequence encoded by a gene of a human V gene group selected from the group consisting of IGHV1-18, IGHV1-2, IGHV1-24, IGHV1-3, IGHV1-45, IGHV1-46, IGHV1-58, IGHV1-69, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-43, IGHV3-43D, IGHV3-48, IGHV3-49, IGHV3-54, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-1, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, and IGHV7-4-1. Optionally, a VH region of an antibody of the disclosure comprises a VH comprising an amino acid sequence (e.g. CDR(s) and/or a human framework region(s), for example according to Kabat numbering) from said gene.

In one embodiment, a VL region of an antibody of the disclosure comprises an amino acid sequence having at least about 80%, 85%, 90%, 95%, 97%, 98% or 99% identity, to the amino acid sequence encoded by a gene of a human V gene group selected from the group consisting of IGKV1-12, IGKV1-13, IGKV1-16, IGKV1-27, IGKV1-33, IGKV1-39, IGKV1-5, IGKV1-6, IGKV1-8, IGKV1-9, IGKV1-NL1, IGKV1D-12, IGKV1D-13, IGKV1D-16, IGKV1D-17, IGKV1D-33, IGKV1D-39, IGKV1D43, IGKV1D8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21 and IGKV6D-21. Optionally, a VL region of an antibody of the disclosure comprises a VL comprising an amino acid sequence (e.g. CDR(s) and/or a human framework region(s), for example according to Kabat numbering) from said gene.

An exemplary anti-Nectin-4 VH and VL pair according to the disclosure is that of antibody 5E7, the amino acid sequence of the heavy chain variable region of which is listed below (SEQ ID NO: 6), and the amino acid sequence of the light chain variable region of which is listed below (SEQ ID NO: 7). In a specific embodiment, provided is an antibody that binds essentially the same epitope or determinant as monoclonal antibody 5E7; optionally the antibody comprises the hypervariable region of antibody 5E7. In any of the embodiments herein, antibody 5E7 can be characterized by the amino acid sequences and/or nucleic acid sequences encoding it. In one embodiment, the monoclonal antibody comprises the Fab or F(ab′)₂ portion of 5E7. Also provided is an antibody or antibody fragment that comprises the heavy chain variable region of 5E7. According to one embodiment, the antibody or antibody fragment comprises the three CDRs of the heavy chain variable region of 5E7. Also provided is an antibody or antibody fragment that further €prises the variable light chain variable region of 5E7 or one, two or three of the CDRs of the light chain variable region of 5E7. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In one aspect, an anti-Nectin-4 antibody or antibody fragment comprises a V_{H} domain having at least about 60%, 70% or 80% sequence identity, optionally at least about 85%, 90%, 95%, 97%, 98% or 99% identity, to the VH domain of SEQ ID NO: 6. In another aspect, the anti-Nectin-4 antibody comprises a V_{L} domain having at least about 60%, 70% or 80% sequence identity, optionally at least about 85%, 90%, 95%, 97%, 98% or 99% identity, to the VL domain of SEQ ID NO: 7.

Optionally, the VH and VL comprise (e.g., are modified to incorporate) human acceptor frameworks. In one embodiment, an anti-Nectin-4 antibody of the disclosure comprises the VH CDR1, CDR2 and/or CDR3 (e.g., according to Kabat numbering) of the heavy chain variable region having the amino acid sequence of SEQ ID NO: 6. In one embodiment, an anti-Nectin-4 antibody of the disclosure comprise the VL CDR1, CDR2 and/or CDR3 (e.g., according to Kabat numbering) of the light chain variable region having the amino acid sequence of SEQ ID NO: 7. In one embodiment, an anti-Nectin-4 antibody of the disclosure comprises a VH comprising the Kabat CDR1, CDR2 and/or CDR3 of the heavy chain variable region having the amino acid sequence of SEQ ID NO: 6 and a VL comprising a Kabat CDR1, CDR2 and/or CDR3 of the light chain variable region having the amino acid sequence of SEQ ID NO: 7.

| 5E7 VH: | |
|---|---|
| (SEQ ID NO: 6) | |
| QVQLQQPGAELVKPGASVKLSCKASGYIFTSYWMHWVKQRPGQGLEWIGEIDPSDSYTNYNQ | |
| KEKGKATLTLDKSSSTTYMQLSSLTSEDSAVYYCVRGYGNYGDYWGQGTTLTVSS | |
| 5E7 VL: | |
| (SEQ ID NO: 7) | |
| DVVMTQAAFSNPVTLGTSASISCRSSKSLLHSNGITYLYWYLQKPGQSPQLLIYQMSNLASG | |
| VPDRESSSGSGTDFTLRISRVEAEDVGVYYCAQNLELPWTFGGGTKLEIK | |
| 5E7 CDRs: | |
| Kabat: | |
| (SEQ ID NO: 8) | |
| CDR-H1 | SYWMH |
| (SEQ ID NO: 9) | |
| CDR-H2 | EIDPSDSYTNYNQKFKG |
| (SEQ ID NO: 10) | |
| CDR-H3 | GYGNYGDY |
| (SEQ ID NO: 11) | |
| CDR-L1 | RSSKSLLHSNGITYLY |
| (SEQ ID NO: 12) | |
| CDR-L2 | QMSNLAS |
| (SEQ ID NO: 13) | |
| CDR-L3 | AQNLELPWT |
| IMGT: | |
| (SEQ ID NO: 14) | |
| CDR-H1 | GYIFTSYW |
| (SEQ ID NO: 15) | |
| CDR-H2 | IDPSDSYT |
| (SEQ ID NO: 16) | |
| CDR-H3 | VRGYGNYGDY |
| (SEQ ID NO: 17) | |
| CDR-L1 | KSLLHSNGITY |
| (SEQ ID NO: 18) | |
| CDR-L2 | QMS |
| (SEQ ID NO: 13) | |
| CDR-L3 | AQNLELPWT |
| Chothia: | |
| (SEQ ID NO: 19) | |
| CDR-H1 | GYIFTSY |
| (SEQ ID NO: 20) | |
| CDR-H2 | PSDS |
| (SEQ ID NO: 21) | |
| CDR-H3 | YGNYGD |
| (SEQ ID NO: 22) | |
| CDR-L1 | SKSLLHSNGITY |
| (SEQ ID NO: 18) | |
| CDR-L2 | QMS |
| (SEQ ID NO: 23) | |
| CDR-L3 | NLELPW |

In one embodiment, an anti-Nectin-4 antibody may for example comprise a heavy chain variable region comprising the amino acid sequences of SEQ ID NOS: 8-10 and a light chain variable region comprising the amino acid sequences of SEQ ID NOS: 11-13. An anti-Nectin-4 antibody may for example comprise: a HCDR1 comprising an amino acid sequence: SYWMH (SEQ ID NO: 8), or a sequence of at least 4 contiguous amino acids thereof, optionally wherein one or more of these amino acids may be substituted by a different amino acid; a HCDR2 comprising an amino acid sequence: EIDPSDSYTNYNQKFKG (SEQ ID NO: 9), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GYGNYGDY (SEQ ID NO: 10), or a sequence of at least 4, 5 or 6 contiguous amino acids thereof, optionally wherein one or more of these amino acids may be substituted by a different amino acid; a LCDR1 comprising an amino acid sequence: RSSKSLLHSNGITYLY (SEQ ID NO: 11), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: QMSNLAS (SEQ ID NO: 12) or a sequence of at least 4, 5 or 6 contiguous amino acids thereof, optionally wherein one or more of these amino acids may be substituted by a different amino acid; and/or a LCDR3 region comprising an amino acid sequence: AQNLELPWT (SEQ ID NO: 13), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-Nectin-4 VH and VL pair according to the disclosure is that of antibody 10B12, the amino acid sequence of the heavy chain variable region of which is listed below (SEQ ID NO: 24), and the amino acid sequence of the light chain variable region of which is listed below (SEQ ID NO: 25). In a specific embodiment, provided is an antibody that binds essentially the same epitope or determinant as monoclonal antibody 10B12; optionally the antibody comprises the hypervariable region of antibody 10B12. In any of the embodiments herein, antibody 10B12 can be characterized by the amino acid sequences and/or nucleic acid sequences encoding it. In one embodiment, the monoclonal antibody comprises the Fab or F(ab′)₂ portion of 10B12. Also provided is an antibody or antibody fragment that comprises the heavy chain variable region of 10B12. According to one embodiment, the antibody or antibody fragment comprises the three CDRs of the heavy chain variable region of 10B12. Also provided is an antibody or antibody fragment that further comprises the variable light chain variable region of 10B12 or one, two or three of the CDRs of the light chain variable region of 10B12. The HCDR1, 2, 3 and LCDR1, 2, 3 sequences can optionally be specified as all (or each, independently) being those of the Kabat numbering system, those of the Chotia numbering system (e.g. the antibody has a CDR-H1 amino acid sequence GYTFTSY (SEQ ID NO: 26), those of the IMGT numbering (e.g. the antibody has a CDR-H1 amino acid sequence GYTFTSYW (SEQ ID NO: 27), or any other suitable numbering system. Optionally any one or more of said light or heavy chain CDRs may contain one, two, three, four or five or more amino acid modifications (e.g. substitutions, insertions or deletions).

In one aspect, an anti-Nectin-4 antibody or antibody fragment comprises a V_{H} domain having at least about 60%, 70% or 80% sequence identity, optionally at least about 85%, 90%, 95%, 97%, 98% or 99% identity, to the VH domain of SEQ ID NO: 24. In another aspect, the anti-Nectin-4 antibody comprises a V_{L} domain having at least about 60%, 70% or 80% sequence identity, optionally at least about 85%, 90%, 95%, 97%, 98% or 99% identity, to the VL domain of SEQ ID NO: 25.

Optionally, the VH and VL comprise (e.g., are modified to incorporate) human acceptor frameworks. In one embodiment, an anti-Nectin-4 antibody of the disclosure comprises the VH CDR1, CDR2 and/or CDR3 (e.g., according to Kabat numbering) of the heavy chain variable region having the amino acid sequence of SEQ ID NO: 24. In one embodiment, an anti-Nectin-4 antibody of the disclosure comprise the VL CDR1, CDR2 and/or CDR3 (e.g., according to Kabat numbering) of the light chain variable region having the amino acid sequence of SEQ ID NO: 25. In one embodiment, an anti-Nectin-4 antibody of the disclosure comprises a VH comprising the Kabat CDR1, CDR2 and/or CDR3 of the heavy chain variable region having the amino acid sequence of SEQ ID NO: 24 and a VL comprising a Kabat CDR1, CDR2 and/or CDR3 of the light chain variable region having the amino acid sequence of SEQ ID NO: 25.

10B12 VH:
(SEQ ID NO: 24)
QVQLQQPGAELVKPGASVKLSCKASGYTFTSYWMHWVKQRPGQGLEWIGE
IDPSDSYTNYNQKFKGKATLTVDKSSSTAYMQLSSLTSEDSAVYYCVRGY
GNYGDYWGQGTTLTVSS
10B12 VL:
(SEQ ID NO: 25)
DIVMTQAAFSNPVTLGTSASISCRSSKSLLHSNGITYLYWYLQKPGQSPQ
LLIYQMSNLASGVPDRESSSGSGTDFTLRISRVEAEDVGVYYCAQNLELP
WTFGGGTKLEIK

Another exemplary anti-Nectin-4 VH and VL pair according to the disclosure is that of antibody 6C11B, the amino acid sequence of the heavy chain variable region of which is listed below (SEQ ID NO: 42), and the amino acid sequence of the light chain variable region of which is listed below (SEQ ID NO: 43). In a specific embodiment, provided is an antibody that binds essentially the same epitope or determinant as monoclonal antibody 6C11B; optionally the antibody comprises the hypervariable region of antibody 6C11B. In any of the embodiments herein, antibody 6C11B can be characterized by the amino acid sequences and/or nucleic acid sequences encoding it. In one embodiment, the monoclonal antibody comprises the Fab or F(ab′)₂ portion of 6C11B. Also provided is an antibody or antibody fragment that comprises the heavy chain variable region of 6C11B. According to one embodiment, the antibody or antibody fragment comprises the three CDRs of the heavy chain variable region of 6C11B. Also provided is an antibody or antibody fragment that further comprises the variable light chain variable region of 6C11B or one, two or three of the CDRs of the light chain variable region of 6C11B. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In one aspect, an anti-Nectin-4 antibody or antibody fragment comprises a V_{H} domain having at least about 60%, 70% or 80% sequence identity, optionally at least about 85%, 90%, 95%, 97%, 98% or 99% identity, to the VH domain of SEQ ID NO: 42. In another aspect, the anti-Nectin-4 antibody comprises a V_{L} domain having at least about 60%, 70% or 80% sequence identity, optionally at least about 85%, 90%, 95%, 97%, 98% or 99% identity, to the VL domain of SEQ ID NO: 43.

Optionally, the VH and VL comprise (e.g., are modified to incorporate) human acceptor frameworks. In one embodiment, an anti-Nectin-4 antibody of the disclosure comprises the VH CDR1, CDR2 and/or CDR3 (e.g., according to Kabat numbering) of the heavy chain variable region having the amino acid sequence of SEQ ID NO: 42. In one embodiment, an anti-Nectin-4 antibody of the disclosure comprise the VL CDR1, CDR2 and/or CDR3 (e.g., according to Kabat numbering) of the light chain variable region having the amino acid sequence of SEQ ID NO: 43. In one embodiment, an anti-Nectin-4 antibody of the disclosure comprises a VH comprising the Kabat CDR1, CDR2 and/or CDR3 of the heavy chain variable region having the amino acid sequence of SEQ ID NO: 42 and a VL comprising a Kabat CDR1, CDR2 and/or CDR3 of the light chain variable region having the amino acid sequence of SEQ ID NO: 43.

6C11B VH:
(SEQ ID NO: 42)
EVQLQQSGPELVKPGASVKISCKTSGYTFTEYTIHWVKONHGKSLEWIG
RINPKNGDIIHNQNFMGKATLTVDKSSSTAYMELRSLTSEDSAVCYCAR
SGYGSSYGFAYWGQGTLVTVSA
6C11B VL:
(SEQ ID NO: 43)
DIVMTQSPTSLVVSLGQRATISCRASQSVTTPRYSYMHWYQQKPGQPPK
LLIKYASNLESGVPARFSGSGSGTDFTLNIHPVEEEDTATYYCQHSWEI
PYTFGGGTKLEIK
6C11B CDRs:

Kabat:

(SEQ ID NO: 44)

| | |
|---|---|
| CDR-H1 | EYTIH |

(SEQ ID NO: 45)

| | |
|---|---|
| CDR-H2 | RINPKNGDIIHNQNFMG |

(SEQ ID NO: 46)

| | |
|---|---|
| CDR-H3 | SGYGSSYGFAY |

(SEQ ID NO: 47)

| | |
|---|---|
| CDR-L1 | RASQSVTTPRYSYMH |

(SEQ ID NO: 48)

| | |
|---|---|
| CDR-L2 | YASNLES |

(SEQ ID NO: 49)

| | |
|---|---|
| CDR-L3 | QHSWEIPYT |

An anti-Nectin-4 antibody may for example comprise: a HCDR1 comprising an amino acid sequence: EYTIH (SEQ ID NO: 44), or a sequence of at least 4 contiguous amino acids thereof, optionally wherein one or more of these amino acids may be substituted by a different amino acid; a HCDR2 comprising an amino acid sequence: RINPKNGDIIHNQNFMG (SEQ ID NO: 45), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: SGYGSSYGFAY (SEQ ID NO: 46), or a sequence of at least 4, 5 or 6 contiguous amino acids thereof, optionally wherein one or more of these amino acids may be substituted by a different amino acid; a LCDR1 comprising an amino acid sequence: RASQSVTTPRYSYMH (SEQ ID NO: 47), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: YASNLES (SEQ ID NO: 48) or a sequence of at least 4, 5 or 6 contiguous amino acids thereof, optionally wherein one or more of these amino acids may be substituted by a different amino acid; and/or a LCDR3 region comprising an amino acid sequence: QHSWEIPYT (SEQ ID NO: 49), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-Nectin-4 VH and VL pair according to the disclosure is that of antibody 6A7, the amino acid sequence of the heavy chain variable region of which is listed below (SEQ ID NO: 50), and the amino acid sequence of the light chain variable region of which is listed below (SEQ ID NO: 51). In a specific embodiment, provided is an antibody that binds essentially the same epitope or determinant as monoclonal antibody 6A7; optionally the antibody comprises the hypervariable region of antibody 6A7. In any of the embodiments herein, antibody 6A7 can be characterized by the amino acid sequences and/or nucleic acid sequences encoding it. In one embodiment, the monoclonal antibody comprises the Fab or F(ab′)₂ portion of 6A7. Also provided is an antibody or antibody fragment that comprises the heavy chain variable region of 6A7. According to one embodiment, the antibody or antibody fragment comprises the three CDRs of the heavy chain variable region of 6A7. Also provided is an antibody or antibody fragment that further comprises the variable light chain variable region of 6A7 or one, two or three of the CDRs of the light chain variable region of 6A7. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In one aspect, an anti-Nectin-4 antibody or antibody fragment comprises a V_{H} domain having at least about 60%, 70% or 80% sequence identity, optionally at least about 85%, 90%, 95%, 97%, 98% or 99% identity, to the VH domain of SEQ ID NO: 50. In another aspect, the anti-Nectin-4 antibody comprises a V_{L} domain having at least about 60%, 70% or 80% sequence identity, optionally at least about 85%, 90%, 95%, 97%, 98% or 99% identity, to the VL domain of SEQ ID NO: 51.

Optionally, the VH and VL comprise (e.g., are modified to incorporate) human acceptor frameworks. In one embodiment, an anti-Nectin-4 antibody of the disclosure comprises the VH CDR1, CDR2 and/or CDR3 (e.g., according to Kabat numbering) of the heavy chain variable region having the amino acid sequence of SEQ ID NO: 50. In one embodiment, an anti-Nectin-4 antibody of the disclosure comprise the VL CDR1, CDR2 and/or CDR3 (e.g., according to Kabat numbering) of the light chain variable region having the amino acid sequence of SEQ ID NO: 51. In one embodiment, an anti-Nectin-4 antibody of the disclosure comprises a VH comprising the Kabat CDR1, CDR2 and/or CDR3 of the heavy chain variable region having the amino acid sequence of SEQ ID NO: 50 and a VL comprising a Kabat CDR1, CDR2 and/or CDR3 of the light chain variable region having the amino acid sequence of SEQ ID NO: 51.

(SEQ ID NO: 50)
6A7 VH:
QVQLKESGAELVKPGASVKLSCKASGYTFTSYWMHWVKORPGHGLEWVGE
IDPSDSYTNYNQKFKGKATLTIDKSSSTVYMQLSSLTSEDSAVYYCARSG
NYDAMDYWGQGTSVTVSA
6A7 VL:
(SEQ ID NO: 51)
DIVMTQAAFSSAVTLGTSASISCRSSKSLLHSNGITYLYWYLQKPGQSPH
FLIYQMSNLASGVPDRFTSSGSGTDFTLRISRVEAEDVGVYYCAQNLELP
WTFGGGTKLEIK
6A7 CDRs:
Kabat:

| | |
|---|---|
| | (SEQ ID NO: 52) |
| CDR-H1 | SYWMH |
| | (SEQ ID NO: 53) |
| CDR-H2 | EIDPSDSYTNYNQKFKG |
| | (SEQ ID NO: 54) |
| CDR-H3 | SGNYDAMDY |
| | (SEQ ID NO: 55) |
| CDR-L1 | RSSKSLLHSNGITYLY |
| | (SEQ ID NO: 56) |
| CDR-L2 | QMSNLAS |
| | (SEQ ID NO: 57) |
| CDR-L3 | AQNLELPWT |

An anti-Nectin-4 antibody may for example comprise: a HCDR1 comprising an amino acid sequence: SYWMH (SEQ ID NO: 52), or a sequence of at least 4 contiguous amino acids thereof, optionally wherein one or more of these amino acids may be substituted by a different amino acid; a HCDR2 comprising an amino acid sequence: EIDPSDSYTNYNQKFKG (SEQ ID NO: 53), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: SGNYDAMDY (SEQ ID NO: 54), or a sequence of at least 4, 5 or 6 contiguous amino acids thereof, optionally wherein one or more of these amino acids may be substituted by a different amino acid; a LCDR1 comprising an amino acid sequence: RSSKSLLHSNGITYLY (SEQ ID NO: 55), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: QMSNLAS (SEQ ID NO: 56) or a sequence of at least 4, 5 or 6 contiguous amino acids thereof, optionally wherein one or more of these amino acids may be substituted by a different amino acid; and/or a LCDR3 region comprising an amino acid sequence: AQNLELPWT (SEQ ID NO: 57), or a sequence of at least 4, 5, 6, 7 or 8 contiguous amino acids thereof, optionally wherein one or more of these amino acids may be deleted or substituted by a different amino acid. CDR positions may be according to Kabat numbering.

In any aspect, the specified variable region, a FR and/or CDR sequence of an antibody or antibody fragment (e.g. 10B12, 6C11B, 6A7 or 5E7) may comprise one or more sequence modifications, e.g., a substitution (1, 2, 3, 4, 5, 6, 7, 8 or more sequence modifications). In one embodiment, the substitution is a conservative modification.

Fragments and derivatives of antibodies (which are encompassed by the term “antibody” or “antibodies” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F(ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain molecule and (3) single chain polypeptides containing only one heavy chain variable region, or a fragment thereof containing the three CDRs of the heavy chain variable region, without an associated light chain molecule; and multispecific antibodies formed from antibody fragments. Included, inter alia, are a nanobody, domain antibody, single domain antibody or a “dAb”.

An anti-Nectin-4 antibody or antibody fragment (e.g. variable regions and/or CDRs) can optionally be embodied as a multispecific (e.g., bispecific) antibody or protein. For example, a multispecific protein can comprise a hypervariable region (e.g., a VH and a VL) of an antibody of any of the embodiments herein and a hypervariable region (e.g., a VH and a VL) of a different antibody, for example an antibody that binds to an antigen of interest other than Nectin-4).

Also encompassed are antibodies or antibody fragments of the disclosure expressed by a cell, and methods of treatment of cancer that make use of such. For example, a cell expressing a chimeric antigen receptor (CAR) can be constructed. CARs are typically engineered to comprise an extracellular single chain antibody (scFv) fused to the intracellular signaling domain of the T cell antigen receptor complex zeta chain, and have the ability, when expressed in effector cells such as T cells or NK cells, to redirect antigen recognition based on the monoclonal antibody's specificity. In one aspect, provided are genetically engineered immune cells which express and bear on the cell surface membrane a Nectin-4-specific chimeric immune receptor comprising an intracellular signaling domain, a transmembrane domain € and a Nectin-4-specific extracellular domain (e.g., a domain derived from or comprising an antibody or antibody fragment or a variable heavy and light chain regions of the an antibody of the disclosure). Also provided are Nectin-4 specific chimeric immune receptors, DNA constructs encoding the receptors, and plasmid expression vectors containing the constructs in proper orientation for expression.

In one embodiment, the antibody is humanized. “Humanized” forms of antibodies are specific chimeric immunoglobulins, immunoglobulin chains or fragments thereof (such as Fv, Fab, Fab′, F(ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.

The choice of human variable domains, both light and heavy, to be used En making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for Nectin-4 and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. In a one example, the FRs of a humanized antibody chain are derived from a human variable region having at least about 60% overall sequence identity, and preferably at least about 70% or 80% overall sequence identity, with the variable region of the nonhuman donor (e.g., a 5E7, 10B12, 6C11B or 6A7 antibody). Optionally, the humanized heavy and/or light chain variable region shares at least about 60%, 70% or 80% overall sequence identity with the respective heavy and/or light chain variable region of the nonhuman donor (e.g., a 5E7, 10B12, 6C11B or 6A7 antibody). Another method of making “humanized” monoclonal antibodies is to use a XenoMouse (Abgenix, Fremont, CA) as the mouse used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

Advantageously, antibodies of the disclosure can be used in processes for preparing an antibody-conjugate. In one embodiment, a process for preparing an antibody-conjugate comprises conjugating a cytotoxic agent (Z) to an anti-Nectin-4 antibody or antibody fragment of the disclosure. In one embodiment, cytotoxic agent (Z) can be specified as being conjugated to the antibody or fragment via a linker (X). X is a linker which connects the antibody or fragment (Ab) and cytotoxic agent (Z), e.g., upon conjugation X is the residue of a linker following covalent linkage to one or both of Ab and Z.

In embodiments herein, a process for preparing the antibody-drug conjugates comprises a step of contacting and/or reacting an anti-Nectin-4 antibody or antibody fragment of the disclosure (Ab) with a cytotoxic agent (Z). The contacting can be carried out under conditions suitable such that an antibody drug conjugate of an aspect of the disclosure is formed or obtained. Z may for example be comprised in a compound comprising a cytotoxic agent (Z) and a linker (X) or portion of linker (X), such that the step comprises contacting an anti-Nectin-4 antibody or antibody fragment of the disclosure with a compound comprising a cytotoxic agent (Z) and a linker (X) or portion of linker (X). A process can optionally specify a step of isolating or recovering the antibody drug conjugate that is formed, and, optionally, further processing the composition for use as a medicament, optionally formulating said antibody (e.g., with a pharmaceutical excipient) for administration to a human subject.

Optionally, a method of making an ADC comprises conjugating the antibody or antibody fragment to 2, 3, 4, 5, 6, 7 or 8 molecules of cytotoxic agent. Optionally, the composition obtained is characterized by a DAR of between 2 and 4, between 4 and 6, between 6 and 8. Optionally, the method comprises conjugating the antibody to 4 molecules of cytotoxic agent. Optionally, the method further comprises assessing the DAR, and if the DAR corresponds to a pre-determined specification (e.g. a DAR or DAR range as disclosed herein, a DAR of about 2, 4, 6, or 8, etc.), further processing the composition for use as a medicament, optionally formulating said antibody (e.g., with a pharmaceutical excipient) for administration to a human subject.

In some embodiments, the linker (X)-(Z) elements are prepared and isolated prior to contacting (and reacting) the compound comprising (X) and (Z) with the (Ab), thereby forming the antibody drug conjugate.

In some embodiments, the method comprises:
(a) contacting and/or reacting linker (X) or a portion of linker (X) with the (Ab) to form a Ab-X conjugate, and
(b) contacting and/or reacting Ab-X of step (a) with a cytotoxic agent (Z) or a compound comprising a second portion of linker (X) and (Z), thereby forming the antibody drug conjugate.

X can for example represent a molecule comprising a moiety that is cleavable, e.g., under physiological conditions, optionally under intracellular conditions. In one embodiment, X represents a molecule comprising (i) a spacer (Y), (ii) a cleavable moiety and (iii) an optional self-eliminating or non-self-eliminating spacer system (Y′). The cleavable moiety can for example be an oligopeptide (e.g. a di-, tri-, tetra- or penta-peptide). The spacer Y can be positioned between the Ab and the cleavable moiety, and the spacer system (Y′) can be positioned between the cleavable moiety and Z.

In some embodiments, linker X or spacer Y can optionally be specified as comprising a reactive group € capable of reacting (e.g. under suitable conditions, optionally after deprotection) with an amino acid of the antibody or with a complementary reactive group (R′) that is attached to an amino acid of the antibody. Optionally, R is a group reactive with a free amino, hydroxyl, sulfhydryl or carboxyl group on the antibody. For example R can be a maleimid-N-yl group having the structure below, which is reactive with a thiol group (also referred to a sulfhydryl group) on the antibody:

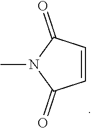

In some embodiments, linker X or spacer Y can optionally be specified as comprising the residue of the reaction of reactive group R with an amino acid of the antibody or with a complementary reactive group (R′) that is attached to an amino acid of the antibody. Optionally, R is the residue of the reaction of a group reactive with a free amino, hydroxyl, sulfhydryl or carboxyl group on the antibody and said free amino, hydroxyl, sulfhydryl or carboxyl group.

In any embodiment, prior to the step of contacting and/or reacting an anti-Nectin-4 antibody or antibody fragment with a compound (e.g. linker and/or cytotoxic agent), the method comprises a step of preparing, selecting or providing an anti-Nectin-4 antibody or antibody fragment. In one embodiment, the step comprise preparing, selecting or providing an anti-Nectin-4 antibody or antibody fragment and determining or testing whether the antibody or antibody fragment has feature(s) of an anti-Nectin-4 antibody or antibody fragment of the disclosure.

For example, an anti-Nectin-4 antibody or antibody fragment can be tested for the ability to bind to the VC1 bridging domain of Nectin-4. An antibody or antibody fragment that is determined to bind to the VC1 bridging domain of Nectin-4 is then contacted and/or reacted with the compound (e.g. linker (X) and/or cytotoxic agent (Z)). For example, an anti-Nectin-4 antibody or antibody fragment can be tested for the ability to bind to a mutant Nectin-4 polypeptide (e.g. a mutant Nectin-4 polypeptide comprising a substitution at residues Q234, K197 and/or S199). An antibody or antibody fragment that is determined to have decreased or loss of binding to the mutant Nectin-4 polypeptide (e.g. compared to binding a wild-type Nectin-4 polypeptide) is then contacted and/or reacted with the compound (e.g. linker (X) and/or cytotoxic agent (Z)).

In another example, an anti-Nectin-4 antibody or antibody fragment can be tested for the ability to reduce cell-cell adhesion between Nectin-4 expressing tumor cells (e.g. adherent tumor cells) and/or for the ability to reduce growth of Nectin-4 expressing tumor cells (e.g. in 3-dimensional cell culture, in a tumor spheroid formation assay). An antibody or antibody fragment that is determined to have the ability to reduce cell-cell adhesion between Nectin-4 expressing tumor cells and/or for the ability to reduce growth of Nectin-4 expressing tumor cells (e.g. adherent tumor cells) is then contacted and/or reacted with the compound (e.g. linker (X) and/or cytotoxic agent (Z)).

In another example, an anti-Nectin-4 antibody or antibody fragment can be tested for the ability to sensitize a tumor to a cytotoxic agent (e.g. a cytotoxic agent Z or of the same class of drugs as Z, for example a camptothecin agent). An antibody or antibody fragment that is determined to have the ability to sensitize a tumor to the cytotoxic agent is then contacted and/or reacted with the compound (e.g. linker (X) and/or cytotoxic agent (Z)).

Optionally, a method can comprise any two or more of the antibody testing steps prior to contacting the antibody with the compound (e.g. linker (X) and/or cytotoxic agent (Z)).

As further described herein, some well-known methods for conjugating cytotoxic agents to antibodies involve multiple reactions steps in which an antibody is first modified with a linker or part of a linker, followed by a reaction in which the cytotoxic agent is conjugated to the antibody-linker composition.

In one embodiment, provided is a process for preparing the antibody-conjugate comprising:
(i) obtaining anti-Nectin-4 antibodies by culturing an host cell transformed with an expression vector containing a polynucleotide encoding the antibody; collecting and purifying the antibody of interest from the cultures obtained in the preceding step,
(ii) contacting an anti-Nectin-4 antibody or antibody fragment with a compound (L) comprising (a) a first reactive group capable of reacting with an amino acid (e.g., a side chain or glycan of the amino acid, or a group attached to an amino acid or glycan of the amino acid) of the antibody and (b) a second reactive group (R′), to obtain a modified antibody comprising one or more amino acids functionalized with the compound (L); and
(iii) reacting the modified antibody of step (i) with a compound comprising (a) a reactive group € that is complementary to reactive group (R′), (b) an amino acid unit (e.g. a di-, tri-, tetra- or penta-peptide) that is cleaved by an intracellular peptidase or protease enzyme, (c) optionally a non-self-eliminating or a self-eliminating spacer (Y′), and (d) a cytotoxic agent (Z). Optionally, the compound of step (ii) further comprises a spacer (Y) placed between R and the amino acid unit.

Optionally, the step obtaining anti-Nectin-4 antibodies can further comprises steps of preparing antibody-producing cells by immunizing an animal by injection of the antigen, collecting the blood, assaying its antibody titer to determine when the spleen is excised; preparing myeloma cells; fusing the antibody-producing cells with the myeloma; screening a group of hybridomas producing a desired antibody; dividing the hybridomas into single cell clones (cloning); culturing the hybridoma or rearing an animal implanted with the hybridoma for producing a large amount of monoclonal antibody; and/or examining the thus produced monoclonal antibody for biological activity and binding specificity, or assaying the same for properties as a labeled reagent; and the like.

Optionally, the step obtaining anti-Nectin-4 antibodies can further comprise steps of preparing a cell library.

In one embodiment, R and R′ are capable of undergoing a click reaction or a cycloaddition, optionally wherein R comprises or is an alkyne moiety and R′ comprises or is an azide moiety, or wherein R′ comprises or is an alkyne moiety and R comprises or is an azide moiety, and wherein the reaction of step (ii) is a 1,3-dipolar cycloaddition.

In one embodiment, the reaction of step (i) is carried out in presence of a catalyst, optionally the catalyst is an enzyme (e.g., a transglutaminase).

In one embodiment, prior to contacting an anti-Nectin-4 antibody or antibody fragment with a compound (L), step (i) comprises a step of modifying the anti-Nectin-4 antibody or antibody fragment. For example, the antibody or antibody fragment can be modified by reacting or contacting it with an enzyme capable of modifying antibody glycosylation (e.g., at Kabat residue N297). In one example, the modification comprises the deglycosylation of an antibody glycan having a core N-acetylglucosamine, in the presence of an endoglycosidase, in order to obtain an antibody comprising a core N-acetylglucosamine substituent, wherein said core N-acetylglucosamine and said core N-acetylglucosamine substituent are optionally fucosylated. Examples of endoglycosidases include EndoS, EndoA, EndoE, Endo18A, EndoF, EndoM, EndoD, EndoH, EndoT and EndoSH and/or a combination thereof.

The antigen binding protein (e.g. antibody) molecule and cytotoxic agent (e.g. camptothecin derivative molecule) are connected by means of a linker. In such embodiments, the immunoconjugate can for example be represented by Formula (II):

Ab-(X—(Z)ₙ)ₘ Formula (II)
wherein,
Ab is an anti-Nectin-4 antibody or antibody fragment);
X is a linker which connects Ab and Z, e.g., the residue of a linker following covalent linkage to one or both of Ab and Z;
Z a cytotoxic agent, e.g. camptothecin analogue, optionally Z comprises a structure of Compounds 1 or 2 (exatecan or a SN-38 molecule);
n is 1 or 2; and
when n is 1, m is from among 1 to 8, or optionally m is an integer selected from among 1 to 8 or 1 to 6, optionally m is an integer selected from among 1 to 4, optionally m is 2 or 4; optionally, m is 2, 3, 4, 5, 6, 7 or 8; and when n is 2, m is from among 1 to 4, or optionally m is an integer selected from among 1 to 4 or 1 to 3, optionally m is an integer selected from among 1 to 4, optionally m is 2 or 4; optionally, m is 1, 2, 4 or 4. Optionally, “n” can be specified to represent the degree of branching or polymerization. “n” and “m” can be specified to represent the average in a composition comprising a plurality of antibodies.

In one embodiment, X represents a molecule comprising a moiety that is cleavable, e.g., under physiological conditions, optionally under intracellular conditions. In one embodiment, X represents a molecule comprising (i) a spacer (Y), (ii) a cleavable moiety and (iii) an optional self-eliminating or non-self-eliminating spacer system (Y′). The spacer Y can be positioned between the Ab and the cleavable moiety, and the spacer system (Y′) can be positioned between the cleavable moiety and Z. Molecule X or spacer Y can optionally be specified as comprising a reactive group € or the residue of the reaction of reactive group R with an amino acid of the antibody or with a complementary reactive group (R′) that is attached to an amino acid of the antibody.

The variable m represents the number of —X—(Z)ₙ moieties per antibody molecule in an immunoconjugate. In a composition comprising a plurality of anti-Nectin-4 ADCs, the number “m” of number of —X—Z moieties per antibody molecule may vary. Thus, in exemplary compositions comprising a plurality of immunoconjugates of the formulae herein, m is the average number of —X—(Z), moieties per Ab, in which case m can also be referred to as the average drug loading or drug:antibody ratio (DAR). Average drug loading or DAR may advantageously range from 1 to about 8 (—X—(Z) n) moieties per Ab. The number of Z moieties attached to a moiety X, “n”, can for example be 1 or 2. Typically, n is 1. In some embodiments, n is 1, and m represents the average drug loading, m is between 2 and 8. In some embodiments, n is 1, and m represents the average drug loading, m is between 2 and 6. In some embodiments, n is 1, and m represents the average drug loading, m is between 4 and 8. In some embodiments, n is 1, and m represents the average drug loading, m is between 6 and 8, optionally about 6, 7 or 8. In some embodiments, n is 1, and m represents the average drug loading, m is between 4 and 6, optionally about 4, 5 or 6.

The number of (—X—Z) moieties per Ab may be characterized by conventional means such as mass spectroscopy, ELISA assay, and HPLC. The quantitative distribution of immunoconjugates in terms of m may also be determined. In some instances, separation, purification, and characterization of homogeneous immunoconjugates where m is a certain value, as distinguished from immunoconjugates with other drug loadings, may be achieved by means such as reverse phase HPLC or electrophoresis.

In one embodiment, an anti-Nectin-4 composition used in the treatment methods of the disclosure is characterized as comprising a plurality immunoconjugates represented by Formula (I):

Ab-(X—(Z)ₙ)ₘ Formula (II)
wherein,
Ab is an anti-Nectin-4 antigen binding protein (e.g. an antibody or antibody fragment);
X is a molecule which connects Ab and Z, e.g., the residue of a linker following covalent linkage to one or both of Ab and Z;
Z is a cytotoxic agent, optionally a topoisomerase inhibitor, optionally a camptothecin analogue, optionally a camptothecin analogue comprising an exatecan or a SN-38 molecule, e.g., a molecule having the structure of Compounds 1 or 2;
n is 1 or 2; and
wherein at least 50%, 60%, 70%, 80%, 90%, 95%, 98% or 99% of immunoconjugates in an antibody sample have an m (the number of X—Z moieties) that is 2 or 4, at least 2, between 2 and 4, at least 4, between 4 and 6 or between 4 and 8, optionally wherein n is 1 and at least 50%, 60%, 70%, 80%, 90%, 95%, 98% or 99% of immunoconjugates in an antibody sample have an m (the number of X—Z moieties) that is 2 or 4, at least 2, between 2 and 4, at least 4, between 4 and 6 or between 4 and 8.

In one embodiment, an anti-Nectin-4 composition used in the treatment methods of the disclosure is characterized as comprising a plurality immunoconjugates represented by Formula (I):

Ab-(X—(Z)ₙ)ₘ Formula (II)
wherein,
Ab is an anti-Nectin-4 antigen binding protein (e.g. an antibody or antibody fragment);
X is a molecule which connects Ab and Z, e.g., the residue of a linker following covalent linkage to one or both of Ab and Z;
Z a camptothecin analogue comprising an exatecan or a SN-38 molecule, e.g., a molecule comprising the structure of Compounds 1 or 2;
n is 1; and
wherein at least 50%, 60%, 70%, 80%, 90%, 95%, 98% or 99% of immunoconjugates in an antibody sample have an m (the number of X—Z moieties) that is 6, at least 6, between 6 and 8, or 8.

It will be appreciated that a variety of methods can be used to covalently link the linker comprising the cytotoxic agent to the antibody or antigen binding protein, either non-specifically or specifically to a particular amino acid residue. The linker (X) can comprise a moiety that is cleavable, e.g., under physiological conditions, optionally as shown in the Examples under intracellular conditions, such that cleavage of the linker releases the cytotoxic agent (e.g. Compound 1, Compound 2, Compound 13, etc.) in the intracellular environment. The linker can be bonded to a chemically reactive group on the antibody molecule, e.g., to a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues), to a carbohydrate, or generally to any reactive group introduced or engineered into an antibody. The site to which the linker is bound can be a natural residue in the amino acid sequence of the antibody molecule or it can be introduced into the antibody molecule, e.g., by DNA recombinant technology (e.g., by introducing a cysteine or protease cleavage site in the amino acid sequence, by introducing a non-natural amino acid residue) or by protein biochemistry (e.g., reduction, pH adjustment or proteolysis, by glycoengineering, enzymatic modification of an amino acid-bound glycan).

In some embodiments, the linker (X) comprises peptide residues comprising amino acid selected from phenylalanine, glycine, valine, lysine, citrulline, serine, glutamic acid, and aspartic acid, optionally the linker (X) comprises a tetrapeptide residue.

In certain embodiments, an intermediate, which is the precursor of the linker (X), is reacted with the cytotoxic agent (Z) under appropriate conditions. In certain embodiments, reactive groups are used on the cytotoxic agent and/or the intermediate. In some embodiments, the product of the reaction between the cytotoxic agent and the intermediate, or the derivatized cytotoxic agent, is subsequently reacted with the antibody molecule under appropriate conditions. In other embodiments, a precursor of the linker (X) is first reacted with the antibody molecule under appropriate conditions so at to yield an antibody bound to the precursor of the linker (X), and the antibody is subsequently reacted with a molecule comprising the cytotoxic agent (Z).

In some embodiments, the linker (X) is cleavable by a cleaving agent that is present in the intracellular environment (e.g., within a lysosome or endosome or caveolea). The linker can comprise for example a peptidyl linker or amino acid unit that is cleaved by an intracellular peptidase or protease enzyme, including, but not limited to, a lysosomal or endosomal protease. In some embodiments, a peptidyl linker moiety is at least two amino acids long or at least three amino acids long. Cleaving agents can include cathepsins B and D and plasmin, all of which are known to hydrolyze dipeptide drug derivatives resulting in the release of active drug inside target cells. Most typical are peptidyl linkers that are cleavable by enzymes that are present in cells. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the valine-citrulline linker). A valine-citrulline (Val-Cit) element can have the structure shown below:

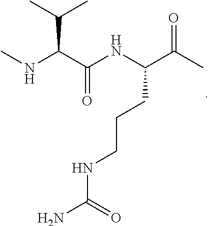 [SMILES: CN[C@H](C(=O)N[C@@H](CCCNC(N)=O)C(C)=O)C(C)C]

In another specific embodiment, the peptidyl linker cleavable by an intracellular protease is a valine-alanine (Val-Ala) linker. A val-ala element can have the structure is shown below:

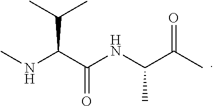

In another specific embodiment, the peptidyl linker cleavable by an intracellular protease is a glycine-containing oligopeptide linker, for example glycine- and phenylalanine-containing oligopeptide linker, optionally a GGF, DGGF, (D-) D-GGF, EGGF, SGGF, KGGF, DGGFG, DDGGFG, KDGGFG, GGFGGGF GGFG, GGFGG or GGFGGG linker (see, e.g., U.S. Pat. No. 6,835,807, the disclosure of which is incorporated herein by reference), wherein “(D-) D represents D-aspartic acid.

In some embodiments, a linker can function to act as a spacer or stretcher to distance an antibody from Z in order to avoid interference with the ability of the antibody to bind Nectin-4 and/or inhibit cell-cell interactions mediated by Nectin-4. A linker may comprise a spacer unit (Y) and/or a spacer or spacer system (Y′). The spacer Y can thus be positioned between the Ab and the cleavable moiety. The spacer system (Y′) can be positioned between the cleavable moiety and Z. Molecule X or spacer Y can optionally be specified as comprising a reactive group € or the residue of the reaction of reactive group R with an amino acid of the antibody or with a complementary reactive group (R′) that is attached to an amino acid of the antibody. The spacer Y can for example be a molecule that forms a bond (e.g. via its reactive group R) with an amino acid of the antibody, e.g., a sulfur atom, a primary or secondary amino group or a carbohydrate group of the antibody, and which spacer or stretcher (Y) links the antibody to the cytotoxic agent (Z) or to a cleavable amino acid unit (e.g. the peptidyl linker, a cleavable di-, tri-, tetra- or penta-peptide, optionally further with a self-eliminating and/or non-self-eliminating spacer (Y′) which is in turn linked to Z. Thus, when the spacer (Y) is linked at one end to an amino acid unit (e.g. a cleavable di-, tri-, tetra- or penta-peptide), the cleavable amino acid unit can in turn be directly linked to Z or can comprise a further spacer (Y′) such as a non-self-eliminating or a self-eliminating spacer which links the amino acid unit and Z.

Spacer (Y) can optionally be specified as being or comprising a substituted or unsubstituted alkyl or heteroalkyl chain, optionally wherein Y has a chain length of 2-100 atoms, optionally 2-40, 2-30, 2-20, 4-40, 4-30 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide.

Spacer (Y) can optionally be specified as comprising a stability-enhancing moiety. For example, spacer Y can orthogonal poly-ethyleneglycol (PEG) moieties or polysarcosine (poly-N-methylglycine or PSAR) moieties in the linker design (see, e.g., WO2019/081455, WO2015/057699 and WO2016/059377, the disclosure of which are incorporated herein by reference).

In some specific embodiments, spacer (Y) may comprise one or more ethylene oxide monomers, optionally Y comprises a polyethylene oxide moiety, optionally Y comprises between 1 and 24, optionally 1 and 12, optionally 1 and 8, optionally 1 and 6 polyethylene oxide moieties, optionally Y comprises a structure —(CH₂CH₂O)ₓ— where x is 1 to 12, optionally 1 to 8, optionally 1 to 6.

An example of a suitable stability-enhancing moiety, spacer chain Y can comprise a stability-enhancing moiety disclosed in PCT publication nos. WO2015/057699 or WO2019/081455. For example, spacer chain Y can comprise an orthogonal connector moiety and stability-enhancing moiety. The stability-enhancing moiety can be a PEG homopolymer, or generally any single molecular weight homopolymer (e.g. a PEG or polysarcosine homopolymer) bound to the orthogonal connector moiety. The homopolymer can have for example 1-4, 1-6, 1-8, 1-10, 1-12, at least 6, 8 or 10, or 6-12, 6-24, 6-72 units of the PEG or other monomer. The term orthogonal connector refers to a branched linker unit component that connects a linker moiety (e.g. the chain of spacer Y) to a homopolymer unit and via a linker (e.g. a cleavable oligopeptide (Pep) and spacer Y′) to a cytotoxic agent (Z) so that the homopolymer unit is in a parallel configuration (as opposed to a series configuration) in relation to the cytotoxic agent (the homopolymer is in parallel to the Pep-Y′—Z moiety). The orthogonal connector moiety can for example be one or more natural or non-natural amino acids optionally selected from glutamic acid, lysine and glycine. Optionally, the amino acid orthogonal connector moiety is placed at the end of spacer chain Y such that the orthogonal connector moiety amino acid residue is connected, via a peptide bond between the α-carboxyl group of one amino acid to the α-amino group of the other amino acid, to an amino acid residue of the peptidyl linker (e.g. (Pep) in Formula V or VI). Y can for example comprise the result of the reaction of the orthogonal connector moiety with a moiety of Formula D:

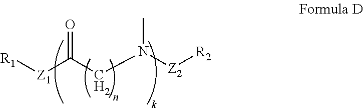

wherein R₁ and R₂ are different, and
one of R₁ and R₂ is H or an inert group, the other one of R₁ and R₂ being functionalized reactive group, said group being reactive for covalently binding to a bindable group of the orthogonal connector moiety, in such reaction conditions that the inert group is non-reactive, Z₁ and Z₂, identical or different, are optional spacers, and n is 1 or more and k is 2 or more.

In another example, spacer Y comprises a group disclosed in US patent publication no. US2017/0072068A1, the disclosures of which are incorporated herein by reference, for example a group according to formula € or a salt thereof:

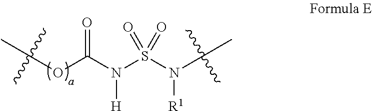

wherein
a is 0 or 1; and
R¹ is selected from the group consisting of hydrogen, C₁-C₂₄ alkyl groups, C₃-C₂₄ cycloalkyl groups, C₂-C₂₄ (hetero) aryl groups, C₃-C₂₄ alkyl(hetero) aryl groups and C₃-C₂₄ (hetero) arylalkyl groups, the C₁-C₂₄ alkyl groups, C₃-C₂₄ cycloalkyl groups, C₂-C₂₄ (hetero) aryl groups, C₃-C₂₄ alkyl(hetero) aryl groups and C₃-C₂₄ (hetero) arylalkyl groups optionally substituted and optionally interrupted by one or more heteroatoms selected from O, S and NR³ wherein R³ is independently selected from the group consisting of hydrogen and C₁-C₄ alkyl groups; and wherein the group according to formula E, or the salt thereof, is situated in between said the first end and the second end of the spacer chain Y.
   According to this aspect, spacer Y can comprise-(Succinimid-3-yl-N)—CH2CH2-C(—O)—, (Succinimid-3-yl-N)—CH2CH2CH2-C(═O)—, -(Succinimid-3-yl-N)—CH2CH2CH2CH2-C(═O)—, -(Succinimid-3-yl-N)—CH2CH2CH2CH2CH2-C(═O)—.

Optionally, such spacer Y can further comprise the following structure: —NH—(CH2CH2-O)n-CH2CH2-C(═O)—, wherein n is an integer of 1 to 6, preferably 2 to 4. Such structure includes for example —NH—CH₂CH₂—O—CH₂CH₂—C(═O)—, —NH—CH₂CH₂—O—CH₂CH₂—O—CH₂CH₂—C(═O)—, —NH—CH₂CH₂—O—CH₂CH₂—O—CH₂CH₂—O—CH₂CH₂—C(═O)—, —NH—CH₂CH₂—O—CH₂CH₂—O—CH₂CH₂—O—CH₂CH₂—O—CH₂CH₂—C(═O)—, —NH—CH₂CH₂—O—CH₂CH₂—O—CH₂CH₂—O—CH₂CH₂—O—CH₂CH₂—O—CH₂CH₂—C(═O)—, —NH—CH₂CH₂—O—CH₂CH₂—O—CH₂CH₂—O—CH₂CH₂—O—CH₂CH₂—O—CH₂CH₂—O—CH₂CH₂—O—CH₂CH₂—C(═O)—.

The spacer or spacer system (Y′) placed between the amino acid unit (e.g. a cleavable di-, tri-, tetra- or penta-peptide) and Z may be self-eliminating or non-self-eliminating. A spacer Y′ may for example comprise a substituted or unsubstituted alkyl or heteroalkyl chain, optionally wherein Y has a chain length of 2-30 atoms, optionally 2-20, 4-20, 2-10 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide. In one embodiment, Y′ comprises a p-aminobenzyloxycarbonyl group. In one embodiment, Y′ is a non-self-eliminating spacer and comprises a structure of —(CH2)n-C(═O)—, wherein n is an integer of 0 to 5, said structure being connected to the cleavable moiety by a —O— or a single bound. For example, Y′ can be or can comprise a —C(═O)—, —O—C(═O)—, —O—CH2-C(═O)—, —O—CH2CH2-C(═O)—, —O—CH2CH2CH2-C(═O)—, —O—CH2CH2CH2CH2-C(═O)—, —O—CH2CH2CH2CH2CH2-C(═O)—, HO—O—CH2-C(═O)—, —CH2-C(═O)—, —CH2CH2-C(═O)—, —CH2CH2CH2-C(═O)—, —CH2CH2CH2CH2-C(═O)—, —CH2CH2CH2CH2CH2-C(═O)—, —CH2-O—CH2-C(═O)— or a —CH2CH2-O—CH2-C(═O)— group.

A “self-eliminating” spacer unit allows for release of the drug moiety without a separate hydrolysis step. When a self-eliminating spacer is used, after cleavage or transformation of the amino acid unit, the side of the spacer linked to the amino acid unit becomes unblocked, which results in eventual release of one or more moieties Z. The self-elimination spacer systems may for example be those described in WO02/083180 and WO2004/043493, the disclosures of which are incorporated herein by reference in their entirety, as well as other self-elimination spacers known to a person skilled in the art. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. In one embodiment, the spacer unit is a p-aminobenzyloxycarbonyl (PAB) for example having the structure:

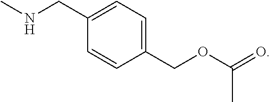

Examples of self-eliminating spacer units further include, but are not limited to, aromatic compounds that are electronically similar to p-aminobenzyl alcohol (see, e.g . . . . US 2005/0256030 A1), such as 2-aminoimidazol-5-methanoi derivatives (Hay et al. (1999) Bioorg. Med. Chem. Lett. 9:2237) and ortho- or para-aminobenzylacetals. Spacers can be used mat undergo cyclization upon amide bond hydrolysis, such as substituted and unsubstituted 4-aminobutyric acid amides (Rodrigues et al. Chemistry Biology, 1995, 2, 223) and 2-aminophenylpropionic acid amides (Amsberry, et al., J. Org. Chem., 1990, 55. 5867). Elimination of amine-containing drugs that are substituted at the α-position of glycine (Kingsbury, et al., J. Med. Chem., 1984, 27, 1447) are also examples of self-eliminating spacers. A p-aminobenzyl self-eliminating spacer (e.g. PAB) is particularly suited for use together with a Phe-Lys, Val-Ala or Val-Cit cleavable dipeptide unit (the PAB is placed between the dipeptide and the camptothecin derivative (Z).

A “non-self-eliminating” spacer unit is one in which part or all of the spacer unit remains bound to the moiety Z upon enzymatic (e.g., proteolytic) cleavage of the antibody-moiety-of-interest conjugate. Examples of non-self-eliminating spacer units adapted for use as a spacer between a Gly-Gly-Phe-Gly amino acid unit and an exatecan molecule include, but are not limited to, include —O—CH₂—C(═O)—, HO—O—CH₂—C(═O)—, —CH₂CH₂—C(═O)—, —CH₂CH₂CH₂—C(═O)—, —CH₂—O—CH₂—C(═O)— and —CH₂CH₂—O—CH₂—C(═O)— (e.g., to form a GGFG-CH₂CH₂—O—CH₂—C(═O)— exatecan unit). Use of such a spacer between the GGFG amino acid unit and exatecan results in the release of a molecule having the structure of Compound 13. Other examples of non-self-eliminating spacer units include, but are not limited to, a glycine spacer unit and a glycine-glycine spacer unit. Other known combinations of peptidic spacers susceptible to sequence-specific enzymatic cleavage can be used in a similar manner. For example, enzymatic cleavage of an antibody-moiety-of-interest conjugate containing a glycine-glycine spacer unit by a tumor cell associated protease would result in release of a glycine-glycine-drug moiety from the remainder of the antibody-moiety-of-interest conjugate. In one such embodiment, the glycine-glycine-drug moiety is then subjected to a separate hydrolysis step in the tumor cell, thus cleaving the glycine-glycine spacer unit from the drug moiety.

An exemplary linker-cytotoxic drug moiety (X—Z) can comprise any of the structures shown below in Formulae III and IV.

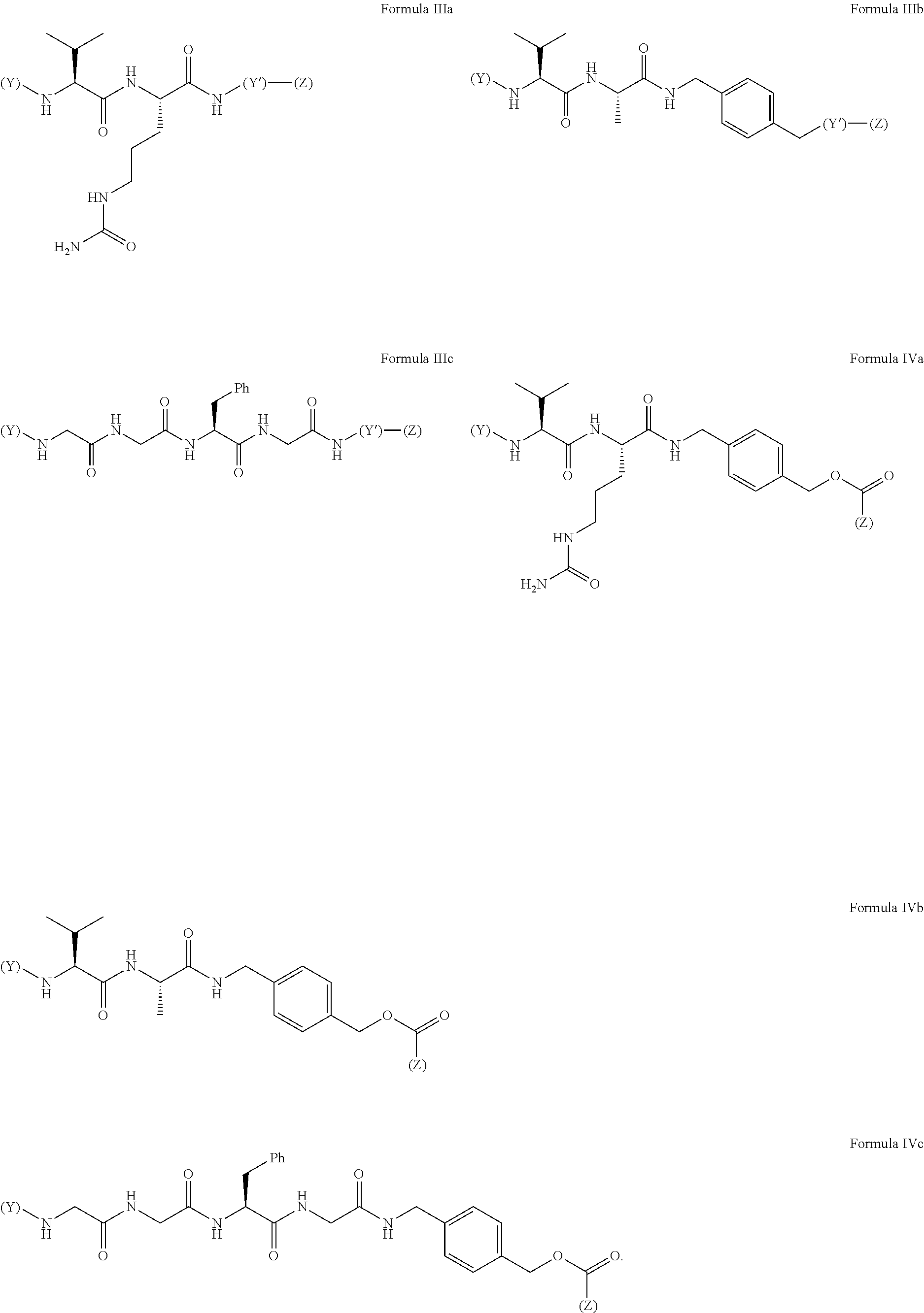

Spacers (Y) and (Y′) can optionally be specified as being are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR1, wherein R¹ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Spacer (Y) and (Y′) can optionally be specified as being or comprising a C₁-C₁₀ alkylene-, —C₁-C₁₀ heteroalkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₅ heterocyclo)-, —(C₃-C₈heterocyclo)-C₁-C₁₀ alkylene-, —C₁-C₁₀ alkylene-C(═O)—, —C₁-C₁₀ heteroalkylene-C(═O)—, —C₃-C₈carbocyclo-C(═O)—, —O—(C₁-C₈alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈carbocyclo)-C(═O)—, —(C₃-C₈carbocyclo)-C₁-C₁₀ alkylene-C(═O)—, —C₃-C₈heterocyclo-C(═O)—, —C₁-C₁₀ alkylene-(C₃-C₈heterocyclo)-C(═O)—, —(C₃-C₈heterocyclo)-C₁-C₁₀ alkylene-C(═O)—, —C₁-C₁₀ alkylene-NH—, —C₁-C₁₀ heteroalkylene-NH—, —C₃-C₈carbocyclo-NH—, —O—(C₁-C₈alkyl)—NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈carbocyclo)-C₁-C₁₀ alkylene-NH—, —C₃-C₈heterocyclo-NH—, —C₁-C₁₀ alkylene-(C₃-C₈heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈carbocyclo-S—, —O—(C₁-C₈ alkyl)-)—S—, -arylene-S—, —C₁-C₁₀ alkylene-arylene-S—, -arylene-C₁-C₁₀ alkylene-S—, —C₁-C₁₀ alkylene-(C₃-C₈carbocyclo)-S—, —(C₃-C₈carbocyclo)-C₁-C₁₀ alkylene-S—, —C₃-C₈ heterocyclo-S—, —C₁-C₁₀ alkylene-(C₃-C₅ heterocyclo)-S—, —(C₃-C₈ heterocyclo)-C₁-C₁₀ alkylene-S—, —C₁-C₁₀ alkylene-O—C(═O)—, —C₃-C₈carbocyclo-O—C(═O)—, —O—(C₁-C₈alkyl)-O—C(═O)—, -arylene-O—C(═O)—, —C₁-C₁₀ alkylene-arylene-O—C(═O)—, -arylene-C₁-C₁₀ alkylene-O—C(═O)—, —C₁-C₁₀ alkylene-(C₃-C₈carbocyclo)-O—C(═O)—, —(C₃-C₈carbocyclo)-C₁-C₁₀ alkylene-O—C(═O)—, —C₃-C₈heterocyclo-O—C(═O)—, —C₁-C₁₀ alkylene-(C₃-C₈heterocyclo)-O—C(═O)—, —(C₃-C₈heterocyclo)-C₁-C₁₀ alkylene-O—C(═O)—, in each case optionally substituted with one or more of the substituents selected from: —X, —R′, —O, —OR′, ═O, —SR′, —S—, —NR′₂, —NR′₃⁺, ═NR′, —CX₃, —CN, —OCN, —SCN, —N═C═O, —NCS, —NO, —NO₂, ═N₂, —N₃, —NR′C(═O) R′, —C(═O) R′, —C(═O) NR′2, —SO₃, —SO₃H, —S(═O)₂R′, —OS(═O)₂OR′, —S(═O)₂NR′, —S(═O)R′, —OP(═O)(OR′)₂, —P(═O)(OR′)₂, —PO₃, —PO₃H₂, —C(═O) X, —C(═S) R′, —CO₂R′, —CO₂, —C(═S) OR′, C(═O) SR′, C(═S) SR′, C(═O) NR′₂, C(═S)NR′₂, and C(═NR′)NR′₂, where each X is independently a halogen: —F, —Cl, —Br, or —I; and each R′ is independently —H, —C₁-C₂₀ alkyl, —C₆-C₂₀ aryl, or —C₃-C₁₄heterocycle.

In one embodiment, spacer (Y) can optionally be specified as comprising, e.g., at one end of the chain, a reactive group (R) that is reactive with a free amino, hydroxyl, sulfhydryl or carboxyl group, or carbohydrate, on the antibody. In some aspects, spacer Y comprises the R group-(Succinimid-3-yl-N)—(CH₂)ₙ³—C(═O), wherein n³ is an integer of 2 to 8, and -(Succinimid-3-yl-N) has a structure represented by the formula F hereinafter:

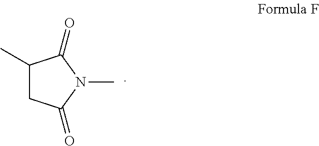

Position 3 of the above structure can be the connecting position to the antibody. The bond to the antibody at position 3 is characterized by bonding with thioether formation.

In one embodiment, spacer (Y) can optionally be specified as comprising, e.g., at one end of the chain, a reactive group (R) that is reactive with a complementary reactive group (R′) that is attached to an amino acid (e.g., via a free amino, hydroxyl, sulfhydryl or carboxyl group, or carbohydrate) of the antibody, or, upon conjugation to an anti-Nectin-4 antibody, the residue of the reaction of a reactive group (R) with a free amino, hydroxyl, sulfhydryl or carboxyl group on the antibody or with a complementary reactive group (R′) that is attached to an amino acid of the antibody. Examples of reactive group pairs R and R′ include a range of groups capable of biorthogonal reaction, preferably a cycloaddition, for example a Diels-Alder reaction or a 1,3-dipolar cycloaddition, for example between azides and cyclooctynes (copper-free click chemistry), between nitrones and cyclooctynes, oxime/hydrazone formation from aldehydes and ketones and the tetrazine ligation (see also WO2013/092983 or US2017/0072068A1, the disclosures of which are incorporated herein by reference). For example, R can be an alkyne and R′ can be an azide, or R can be an azide and R′ an alkyne. The resulting linker and functionalized antibody, or the Y element thereof, can thus in any embodiment comprise a group (RR′) resulting from the reaction of R and R′, for example RR′ can be or comprise a triazole resulting from the reaction of an alkyne and an azide.

In one embodiment, the reactive groups R and R′ are complementary reagents together capable of undergoing a “click” reaction (i.e., a Click Chemistry reagent or reactive group). For example, a 1,3-dipole-functional compound can react with an alkyne in a cyclization reaction to form a heterocyclic compound, preferably in the substantial absence of added catalyst (e.g., Cu(I)). A variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

Examples include o-phosphenearomatic ester, an azide, a fulminate, an alkyne (including any strained cycloalkyne), a cyanide, an anthracene, a 1,2,4,5-tetrazine, or a norbornene (or other strained cycloalkene).

In one embodiment, R is a moiety having a terminal alkyne or azide; such moieties are described for example in U.S. Pat. No. 7,763,736, the disclosure of which is incorporated herein by reference. Suitable reaction conditions for use of copper (and other metal salt) as catalysts of click-reactions between terminal alkynes and azides are provided in U.S. Pat. No. 7,763,736.

In one embodiment, R is a substituted or unsubstituted cycloalkyne. Cycloalkynes, including specific compounds, are described for example in U.S. Pat. No. 7,807,619, the disclosure of which is incorporated herein by reference.

In some embodiments a cycloalkyne may be a compound of Formula A:

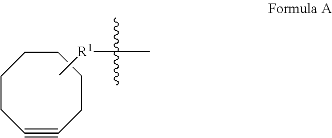

R¹ is selected from a carbonyl, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone, and a halosulfonyl;
R¹ can be at any position on the cyclooctyne group other than at the two carbons joined by the triple bond.

In some embodiments, the modified cycloalkyne is of Formula A, wherein one or more of the carbon atoms in the cyclooctyne ring, other than the two carbon atoms joined by a triple bond, is substituted with one or more electron-withdrawing groups, e.g., a halo (bromo, chloro, fluoro, iodo), a nitro group, a cyano group, a sulfone group, or a sulfonic acid group. Thus, e.g., in some embodiments, a subject modified cycloalkyne is of Formula B:

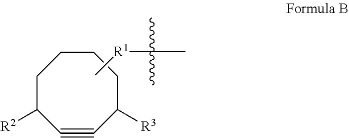

where:
each of R² and R³ is independently: (a) H; (b) a halogen atom (e.g., bromo, chloro, fluoro, iodo); (c) —W—(CH₂)ₙ—Z (where: n is an integer from 1-4 (e.g., n=1, 2, 3, or 4); W, if present, is O, N, or S; and Z is nitro, cyano, sulfonic acid, or a halogen); (d)-(CH₂)ₙ—W—(CH₂)ₘ−R⁴ (where: n and m are each independently 1 or 2; W is O, N, S, or sulfonyl; if W is O, N, or S, then R⁴ is nitro, cyano, or halogen; and if W is sulfonyl, then R⁴ is H); or (e) —CH₂)ₙ—R⁴ (where: n is an integer from 1-4 (e.g., n=1, 2, 3, or 4); and R⁴ is nitro, cyano, sulfonic acid, or a halogen); and

R¹ is selected from a carbonyl, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone and a halosulfonyl. R¹ can be at any position on the cyclooctyne group other than at the two carbons linked by the triple bond.

In one embodiment, R is a substituted or unsubstituted heterocyclic strained alkyne. Cycloalkynes, including specific compounds, are described for example in U.S. Pat. No. 8,133,515, the disclosure of which is incorporated herein by reference. In one embodiment, the alkyne is of the Formula C:

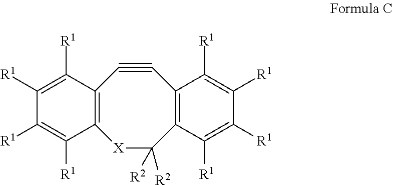

wherein:
each R¹ is independently selected from the group consisting of hydrogen, halogen, hydroxy, alkoxy, nitrate, nitrite, sulfate, and a C₁-C₁₀ alkyl or heteroalkyl;
each R₂ is independently selected from the group consisting of hydrogen, halogen, hydroxy, alkoxy, nitrate, nitrite, sulfate, and a C₁-C₁₀ organic group; X represents N—R³R⁴, NH—R⁴, CH—N—OR⁴, C—N—NR³R⁴, CHOR₄, or CHNHR₄; and each R³ represents hydrogen or an organic group and R⁴ represents linking moiety C of a linker. In one embodiment, R or R′ is a DBCO (dibenzycyclooctyl) group below:

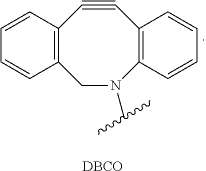 [SMILES: *N1Cc2ccccc2C#Cc2ccccc21 ; "DBCO"]

Alkynes such as those described herein above can be reacted with at least one 1,3-dipole-functional compound in a cyclization reaction to form a heterocyclic compound, preferably in the substantial absence of added catalyst (e.g., Cu(I)). A wide variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

In the Formulae herein, Y′ can be optionally absent or can be a spacer, optionally a self-eliminating spacer, for example comprising p-aminobenzyl unit, or a non-self-eliminating spacer. Optionally, Y′ is or comprises a substituted or unsubstituted alkyl or heteroalkyl chain, optionally wherein Y′ has a chain length of 2-40 atoms, optionally 2-30, 2-20, 4-40, 4-30 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide.

An exemplary linker-cytotoxic agent molecule (e.g. an X—Z moiety of Formulae I to XI) that can be conjugated to an anti-Nectin-4 antibody can optionally be represented by Formula V:

(R)—(Y)-(Pep)-(Y′)—(Z) Formula (VI)
wherein,
R is a group reactive with a free amino, hydroxyl, sulfhydryl or carboxyl group on the antibody, or reactive with a complementary reactive group (R′) that is attached to an amino acid of the antibody, or, upon conjugation to the anti-Nectin-4 binding protein R is the residue of the reaction of a reactive group (R) with a free amino, hydroxyl, sulfhydryl or carboxyl group on the antibody or with a complementary reactive group (R′) that is attached to an amino acid of the antibody;
Y is optionally absent or is a spacer;
Pep is or comprises a peptidyl linker that is cleaved by an intracellular peptidase or protease enzyme, for example a valine-citrulline, valine-alanine or phenylalanine-lysine dipeptide;
Y′ is optionally absent or is a spacer, optionally a self-eliminating spacer or a non-self-eliminating spacer; and
Z is a cytotoxic agent, optionally a camptothecin analogue, optionally an exatecan, Dxd or SN-38 molecule.

A resulting Nectin-4 binding immunoconjugate according to the invention can for example be represented by Formula (VI):

Ab-(Y)-(Pep)-(Y′)-(Z) Formula (VI)
wherein,
Ab is an anti-Nectin-4 an antibody;
Y is optionally absent or is a spacer. Optionally Formula VI comprises, between (Ab) and (Y) the residue of the reaction of a reactive group (e.g. a maleimide, a primary amine) with the side chain or carbohydrate of an amino acid of anti-Nectin-4 antigen binding protein (Ab). Alternatively, the residue of the reaction of a reactive group (e.g. a maleimide, a primary amine) with the side chain of an amino acid of anti-Nectin-4 antigen binding protein (Ab) can be specified as being comprised in Y;
Pep is or comprises an amino acid unit (e.g. peptidyl linker) that is cleaved by an intracellular peptidase or protease enzyme, (e.g., (Pep) is a protease-cleavable di-, tri-, tetra- or penta-peptide, for example a valine-citrulline, valine-alanine or phenylalanine-lysine unit);
Y′ is optionally absent or is a spacer, optionally a self-eliminating spacer or a non-self-eliminating spacer; and
Z is a cytotoxic agent, optionally a camptothecin derivative, optionally an exatecan, Dxd or SN-38 molecule.

Optionally, the formula comprises can be specified as comprising (e.g. between (Ab) and the end of Y (or (Pep or X) if Y is absent)) the residue (RR′) of the reaction of a reactive group (R) with a free amino, hydroxyl, sulfhydryl or carboxyl group on the antibody or with a complementary reactive group (R′) that is attached to an amino acid of the antibody.

In one example, where (RR′) is the residue of the reaction of a reactive group (R) with a complementary reactive group (R′) that is attached to the antibody (e.g. R′ is attached to a side chain or glycan of an amino acid of the antibody), the Nectin-4 binding immunoconjugate according to the invention can for example be represented by Formula (VI_{bis}):

Ab-(RR′)-(Y)-(Pep)-(Y′)-(Z) Formula (VI_{bis})

wherein, Ab, Y, Pep, Y′ and Z are as defined in Formula VI, and RR′ is the result of biorthogonal reaction, preferably a cycloaddition, for example a Diels-Alder reaction or a 1,3-dipolar cycloaddition. In one embodiment, RR′ has a structure selected from the group consisting of:

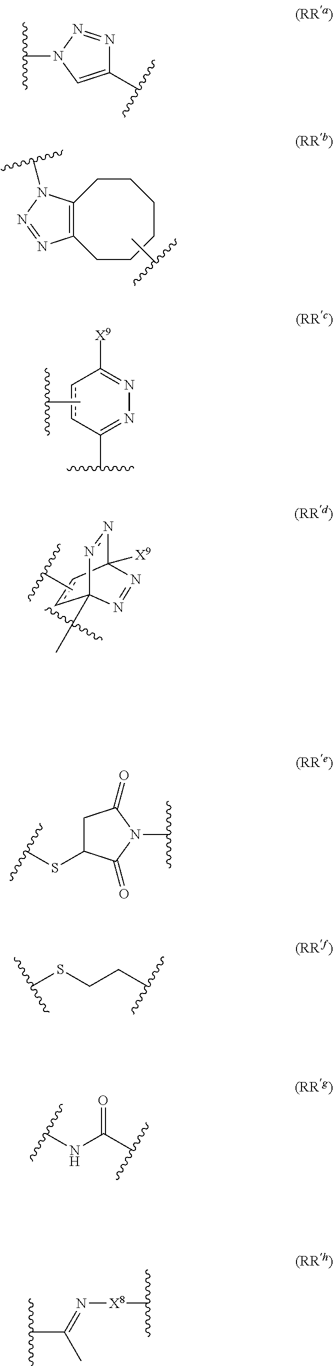

wherein X⁸ is O or NH, X⁹ is selected from H, methyl and pyridyl, and in structure (RR′^{c}) and (RR′^{d}), and the [symbol] bond represents either a single or a double bond.

In any embodiment, an exatecan molecule (or other 6-ring camptothecin) can be specified as being bound to Y′ (or (Pep) if Y′ is absent) via the amine at position 1 of exatecan.

In any embodiment, a SN-38 molecule (or other 5-ring camptothecin) can be specified as being bound to Y′ (or (Pep) if Y′ is absent) via the amine at position 9 of SN-38.

The cytotoxic agent, also referred to aa the (Z) moiety, includes, for example, cytotoxic agents such as antineoplastic agents. Examples of cytotoxic agents are known in the art. For example, Z can be an alkylating agent, preferably a DNA alkylating agent. An alkylation agent is a compound that can replace a hydrogen atom with an alkyl group under physiological conditions (e.g. pH 7.4, 37 C, aqueous solution). Alkylation reactions are typically described in terms of substitution reactions by N, O and S heteroatomic nucleophiles with the electrophilic alkylating agent, although Michael addition reactions are also important. Examples of alkylating agents include nitrogen and sulfur mustards, ethylenimines, methanosulfonates, CC-1065 and duocarmycins, nitrosoureas, platinum-containing agents, agents that effectuate Topoisomerase II-mediated site dependent alkylation of DNA (e.g. psorospermin and related bisfuranoxanthones), ecteinascidin and other or related DNA minor groove alkylation agents.

In one embodiment, Z is a chelated metal, such as chelates of di- or tripositive metals having a coordination number from 2 to 8 inclusive. Particular examples of such metals include technetium (Tc), rhenium (Re), cobalt (Co), copper (Cu), gold (Au), silver (Ag), lead (Pb), bismuth (Bi), indium (In), gallium (Ga), yttrium (Y), terbium (Tb), gadolinium (Gd), and scandium (Sc). In general the metal is preferably a radionuclide. Particular radionuclides include 99mTc, 186Re, 188Re, 58Co, 60Co, 67Cu, 195Au, 199Au, 110Ag, 203Pb, 206Bi, 207Bi, 111 In, 67Ga, 68Ga, 88Y, 90Y, 160Tb, 153Gd and 47Sc. The chelated metal may be for example one of the above types of metal chelated with any suitable polydentate chelating agent, for example acyclic or cyclic polyamines, polyethers, (e.g. crown ethers and derivatives thereof); polyamides; porphyrins; and carbocyclic derivatives.

An anti-Nectin-4 antibody or antibody fragment can also be used in diagnostics, e.g., to detect Nectin-4 tumor cells. In such embodiments, an antibody or antibody fragment can be conjugated to an effector molecules such as detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerytbrin; suitable luminescent materials include luminol; suitable bioluminescent materials include luciferase, luciferin, and aequorin; and suitable radioactive nuclides include 1251, 1311, 111 In and 99Tc.

In one embodiment, cytotoxic agent (Z) is a DNA minor groove binding and/or alkylating agent, e.g., a pyrrolobenzodiazepine, a duocarmycin, or derivatives thereof.

In a further embodiment, the cytotoxic agent is selected from the group consisting of taxanes, anthracyclines, camptothecins, epothilones, mytomycins, combretastatins, vinca alkaloids, nitrogen mustards, maytansinoids, calicheamycins, duocarmycins, tubulysins, dolastatins and auristatins, enediynes, amatoxins, pyrrolobenzodiazepines, ethylenimines, radioisotopes, therapeutic proteins and peptides, and toxins or fragments thereof.

In a further embodiment, the cytotoxic agent is selected from cyclophosphamide, ifosfamide, chlorambucil, 4-(bis(2-chloroethyl)amino) phenol, 4-(bis(2-fluoroethyl) ammo) phenol, N, N-bis(2-chloroethyl)-p-phenylenediamine, N, N-bis(2-fluoro-ethyl)-p-phenylenediamine, carmustine, lomustine, treosulfan, dacarbazine, cisplatin, carboplatin, vincristine, vinblastine, vindesine, vinorelbine, paclitaxel, docetaxel, etoposide, teniposide, topotecan, inirotecan, 9-aminocamptothecin, 9-nitrocamptothecin, 10-hydroxycamptothecin, lurtotecan, camptothecin, crisnatol, mitomycin C, mitomycin A, methotrexate, trimetrexate, mycophenolic acid, tiazofurin, ribavirin, hydroxyurea, deferoxamine, 5-fluorouracil, floxuridine, doxifluridine, raltitrexed, cytarabine, cytosine arabinoside, fludarabine, 6-mercaptopurine, thioguanine, raloxifen, megestrol, goserelin, leuprolide acetate, flutamide, bicalutamide, vertoporfin, phthalocyanine, photosensitizer Pc4, demethoxy-hypocrellin A, interferon-alpha, interferon-gamma, tumor necrosis factor, lovastatin, staurosporine, actinomycin D, bleomycin A2, bleomycin B2, peplomycin, daunorubicin, doxorubicin, N-(5,5-diacetoxypentyl) doxorubicin, morpholino doxorubicin, idarubicin, epirubicin, pirarubicin, zorubicin, mitoxantrone, thapsigargin, N8-acetylspermidine, tallysomycin, esperamycin, butyric acid, retinoic acid, 1,8-dihydroxybicyclo [7.3.1] trideca-4-ene-2,6-diyne-13-one, anguidine, podophyllotoxin, combretastatin A-4, pancratistatin, tubulysin A, tubulysin D, carminomycin, streptonigrin, elliptmium acetate, maytansine, maytansinol, calicheamycin, mertansine (DM1), N-acetyl-γ₁^{I}-calicheamycin, calicheamycin-γ₁^{I}, calicheamycin-α₂^{I}, calicheamycin-α₃^{I}, duocarmycin SA, duocarmycin A, CC-1065, CBI-TMI, duocarmycin C2, duocarmycin centanamycin, dolastatin, auristatin E, monomethylauristatin E (MMAE), monomethylauristatin F (MMAF), α-amanitin, β-amanitin, γ-amanitin, ε-amanitin, amanin, amaninamide, amanullin, and amanullinic acid and derivatives thereof.

Exemplary auristatin embodiments include the N-terminus linked monomethylauristatin drug moieties comprising a structure of any of Formulas (a1) and (a2) below:

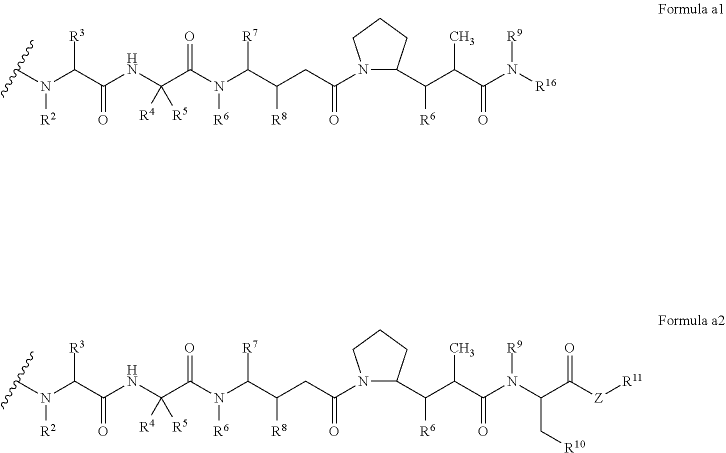

wherein the wavy line of (a1) and (a2) indicates the covalent attachment site to linker (e.g. linker X, or moiety Y, Pep or Y′), and independently at each location:
R² is selected from H and C₁-C₈ alkyl;
R³ is selected from H, C₁-C₈ alkyl, C₁-C₈ carbocycle, aryl, C₁-C₈ alkyl-aryl, C₁-C₈ alkyl-(C₃-C₈ carbocycle), C₃-C₅ heterocycle and C₁-C₈ alkyl-(C₃-C₅ heterocycle);
R⁴ is selected from H, C₁-C₈ alkyl, C₃-C₈ carbocycle. aryl, C₁-C₈ alkyl-aryl, C₁-C₈ alkyl-(C₃-C₅ carbocycle), C₃-C₅ heterocycle and C₁-C₈ alkyl-(C₃-C₈ heterocycle);
R⁵ is selected from H and methyl;
or R⁴ and R⁵ jointly form a carbocyclic ring and have the formula —(CRₐR_{b}), wherein Rₐ and R^{b} are independently selected from H, C₁-C₈ alkyl and C₃-C₈ carbocycle and n is selected from 2, 3, 4, 5 and 6;
R⁶ is selected from H and C₁-C₈ alkyl;
R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
each R⁸ is independently selected from H, OH, C₁-C₈ alkyl, C₃-C₈ carbocycle and O—(C₁-C₈ alkyl);
R⁹ is selected from H and C₁-C₈ alkyl;
R¹⁰ is selected from aryl or C₃-C₈ heterocycle;
Z is O, S, NH, or NR¹² wherein R¹² is C₁-C₈ alkyl;
R¹¹ is selected from H, C₁-C₂₀ alkyl, aryl, C₃-C₈ heterocycle, —(R^{13O})ₘ—R¹⁴, or —(R¹³O)ₘ-CH(R¹⁵)₂; m is an integer ranging from 1-1000;
R¹³ is C₂-C₈ alkyl;
R¹⁴ is H or C₁-C₈ alkyl;
each occurrence of R¹⁵ is independently H, COOH, —(CH₂)ₙ—N(R¹⁶)₂, —(CH₂)ₙ—SO₃-C₁-C₈ alkyl;
each occurrence of R¹⁶ is independently H, C₁-C₈ alkyl, or —(CH₂)ₙ—COOH;
R¹⁸ is selected from —C(R⁸)₂—C(R⁸)₂-aryl, —C(R⁸)₂—C(R⁸)₂—(C₃-C₈ heterocycle), and —C(R⁸)₂-C(R⁸)₂—(C₃-C₈ carbocycle); and
n is an integer ranging from 0 to 6.

In one embodiment, R³, R⁴ and R⁷ are independently isopropyl or sec-butyl and R⁵ is —H or methyl. In an exemplary embodiment. R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is-phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ-N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH²)ₙ—SO₃H.

One exemplary auristatin embodiment of formula (a1) is MMAE, wherein the wavy line indicates the covalent attachment to a linker:

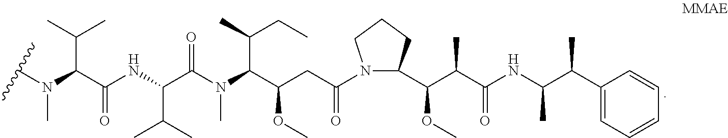 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](C)c1ccccc1)OC)C(C)C)C(C)C]

An exemplary auristatin embodiment of formula (a2) is MMAF, wherein the wavy line indicates the covalent attachment to a linker (L) of an antibody-drug conjugate (see US 2005/0238649 and Doronina et al. (2006) Bioconjugate Cfiem. 17:1 14-124):

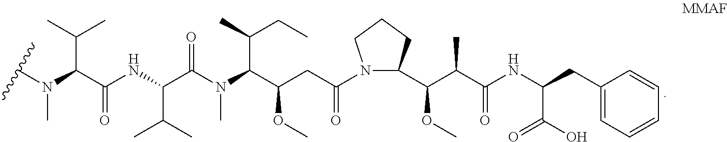 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Other exemplary Z embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Other drug moieties include the following MMAF derivatives, wherein the wavy line indicates the covalent attachment to a linker:

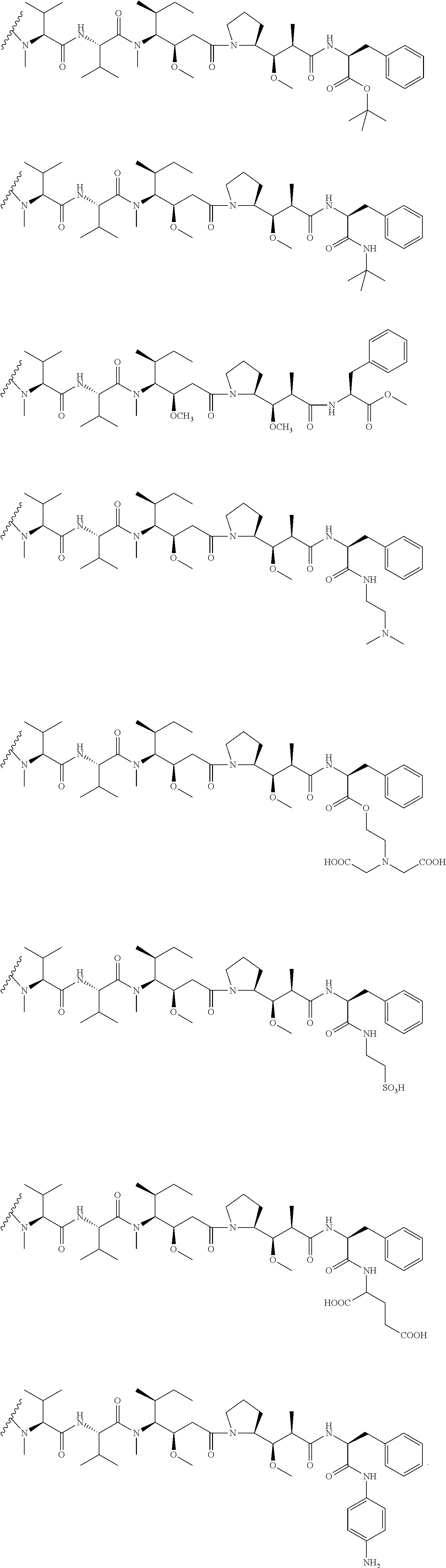

An example of a linker comprising a spacer (Y) comprising a primary amine, a valine-citrulline as the (Pep) moiety, a PAB as the (Y′) moiety together with a MMAF as the (Z) moiety is shown below:

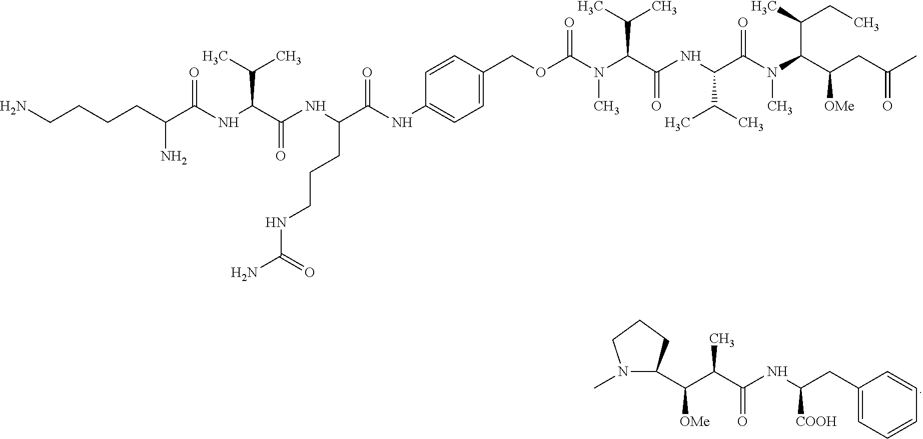

In one embodiment, the Z moiety is a DNA minor groove binding agent, optionally Z comprises a pyrrolobenzodiazepine (PBD). In one embodiment, Z is a pyrrolobenzodiazepine monomer. In one embodiment, Z is a pyrrolobenzodiazepine dimer comprising two pyrrolobenzodiazepine units. In one embodiment, Z is a pyrrolobenzodiazepine trimer comprising three pyrrolobenzodiazepine units. In one embodiment, Z is a pyrrolobenzodiazepine multimer comprising more than three pyrrolobenzodiazepine units. Structures of PBDs, as well as formulas and methods of producing them are described for example in PCT publications Nos: WO 2013/177481, WO 2011/130616, WO 2004/043880, WO 2005/085251, WO2012/112687 and WO 2011/023883, the disclosures of each of which are incorporated herein by reference.

The pyrrolo[2,1-c][1,4] benzodiazepines are a family of sequence-selective, minor-groove binding DNA-interactive agents that covalently attach to guanine residues. It has been reported that the(S)-chirality at the C₁₁a-position of PBDs provides them with the appropriate 3-dimensional shape to fit perfectly into the DNA minor groove. PBDs can have different effects and modes of action. PBDs can be DNA-binders or DNA-alkylators that do not cause crosslinking of DNA, or PBDs can be DNA cross-linkers.

The pyrrolobenzodiazepine unit or monomer can have a general structure as follows:

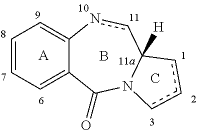

wherein the PBD can have different number, type and position of substituents, in both the aromatic A rings and pyrrolo C rings, and can vary in the degree of saturation of the C ring. In the B-ring there is either an imine (N═C), a carbinolamine (NH—CH(OH)), or a carbinolamine methyl ether (NH—CH(OMe)) at the N¹⁰—C¹¹ position which is the electrophilic centre responsible for alkylating DNA.

The biological activity of PBDs can be potentiated by joining two PBD monomers or units together, typically through their C8/C8′-hydroxyl functionalities via a flexible alkylene linker.

In one aspect of the any of the embodiments herein, a pyrrolobenzodiazepine monomer or unit is a pyrrolo[2,1-c][1,4] benzodiazepine. In one aspect of the any of the embodiments herein, a pyrrolobenzodiazepine dimer is a C8/C8′-linked pyrrolo[2,1-c][1,4]benzodiazepine dimer.

A PBD can be attached to a linker through any suitable position. For example, the PBD can be connected to a linker, via any of the positions in a PBD unit indicated below.

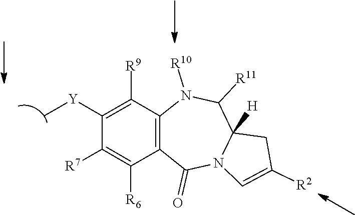

In one embodiment, a PBD dimer comprises the structure of the general formula below, with exemplary attachments points to other substituents or functionalities within a compound indicated by arrows:

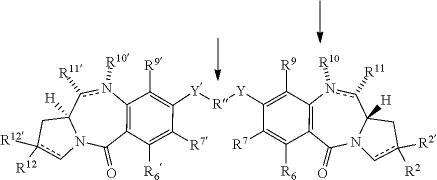

wherein:
R¹² and R^{12′}, and/or R² and R² together respectively form a double bond ═CH₂ or ═CH—CH₃; or
R^{2′} and R^{12′} are absent and R² and R¹² are independently selected from:
(iia) C₁₋₅ saturated aliphatic alkyl;
(iib) C₃₋₆ saturated cycloalkyl;
(iic)

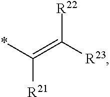

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

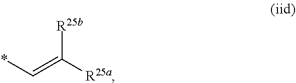

wherein one of R^{25a} and R^{25b} is H and the other is selected from:
phenyl, which phenyl is optionally substituted by a group selected from halo, methyl, methoxy; pyridyl; and thiophenyl; and

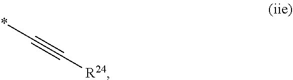

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
R⁶ and R⁹ are independently selected from H, R, OH, OR, SH, SR, NH₂, NHR, NRR′, nitro, Me₃Sn and halo; where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo; either:
(a) R¹⁰ is H, and R¹¹ is OH, OR^{A}, where R^{A} is alkyl;
(b) R¹⁰ and R¹¹ form a nitrogen-carbon double bond between the nitrogen and carbon atoms to which they are bound; or
(c) R¹⁰ is H and R¹¹ is SO_{z}M, where z is 2 or 3 and M is a monovalent pharmaceutically acceptable cation;
R″ is a C₃₋₁₂ alkylene group, which chain may be interrupted by one or more heteroatoms, e.g., O, S, NR^{N2} (where R^{N2} is H or C₁₋₄ alkyl), and/or aromatic rings, e.g., benzene or pyridine;
Y and Y′ are selected from O, S, or NH; and
R^{6′}, R^{7′}, R^{9′} are selected from the same groups as R⁶, R⁷ and R⁹ respectively and R^{10′} and R^{11′} are the same as R¹⁰ and R¹¹, wherein if R¹¹ and R¹¹ are SO_{z}M, M may represent a divalent pharmaceutically acceptable cation.

In another example, a PBD dimer comprises the structure of the general formula below:

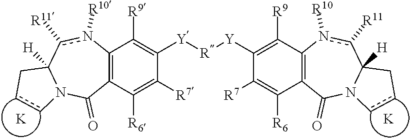

wherein R⁶, R⁷, R⁹, R^{6′}, R^{7′}, R^{9′}, R¹⁰, R¹¹, R^{10′} and R^{11′} are as defined above, and wherein the “K” ring is a substituted or unsubstituted aromatic or non-aromatic ring, optionally a 6-member ring, optionally a phenyl.

In one embodiment, the cytotoxic agent (Z) is a camptothecin, e.g. it has or comprises the structure of camptothecin or of a camptothecin analog. Camptothecin is well known, as are a wide range of camptothecin analogues that share the core ring system with various substitutions, but preferably have modifications or substitutions in rings A and/or B of the basic camptothecin structure below:

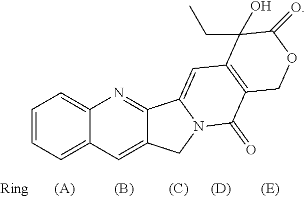

Many camptothecin analogues have been reported including, topotecan, inirotecan, exatecan, DXd, 9-aminocamptothecin, 9-nitrocamptothecin, 10-hydroxycamptothecin, lurtotecan, camptothecin, gimatecan, belotecan, and rubitecan. Further camptothecin analogues are disclosed in Li et al., ACS Med. Chem. Lett. 2019, 10, 10, 1386-1392, Jpn. J. Cancer Res. 86:776-782 and in Takiguchi et al. 1997 Jpn. J. Cancer Res. 88:760-769, the disclosures of which are incorporated herein by reference. The four analogues topotecan, irinotecan, belotecan, and DXd (as part of trastuzumab deruxtecan) have been approved by the FDA. In one embodiment, the camptothecin analogue is a five-ring compound (e.g., the camptothecin lacks an F ring). In one embodiment, the camptothecin analogue is a six-ring compound, e.g., comprising the F ring.

Some examples such as the basic camptothecin structure, the SN-38 molecule (7-Ethyl-10-hydroxycamptothecin; active metabolite of irinotecan) and the camptothecin analogues disclosed in Li et al., ACS Med. Chem. Lett. 2019, 10, 10, 1386-139 have five rings (A, B, C, D and E rings) and can for example be attached to the linker (e.g. spacer Y or Y′, or linker X) via a substituent on the B ring.

SN-38:

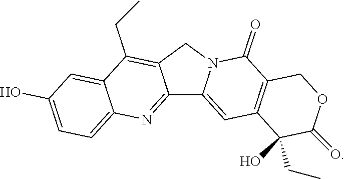 [SMILES: CCc1c2c(nc3ccc(O)cc13)-c1cc3c(c(=O)n1C2)COC(=O)[C@]3(O)CC]

Compounds of Li et al., ACS Med. Chem. Lett. 2019, 10, 10, 1386-139:

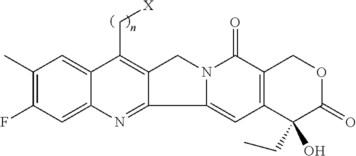

Optionally a camptothecin analogue is a six-ring compound (additionally an F ring), where the compound is attached to the linker through a substituent on such F ring. Examples of such six rings compounds include but are not limited to DXd (CAS No.: 1599440-33-1) and exatecan.

Camptothecin analogues thus include exatecan, SN-38, and any of a range of molecules that comprise such a moiety, for example an exatecan can unsubstituted or can be substituted at the position 1 amine, for example wherein the substituent is or comprises a —C(═O)—, —O—C(═O)—, —O—CH₂—C(═O)—, HO—O—CH₂—C(═O)—, —CH₂CH₂—C(═O)—, —CH₂CH₂CH₂—C(═O)—, —CH₂—O—CH₂—C(═O)—, —CH₂CH₂—O—CH₂—C(═O)— group or other group shown in U.S. Pat. No. 6,835,807, the disclosure of which is incorporated herein by reference.

In one embodiment, the antibody of the disclosure releases, in vivo or in vitro in presence of Nectin-4 expressing tumor cells (e.g. upon enzymatic cleavage of the cleavable moiety followed by self-elimination of the spacer Y′) an exatecan molecule having the structure of Compound 1.

The camptothecin derivative or analogue exatecan is described in Mitsui et al. 1995 Jpn. J. Cancer Res. 86:776-782 and in Takiguchi et al. 1997 Jpn. J. Cancer Res. 88:760-769, the disclosures of which are incorporated herein by reference. The structure of exatecan is shown below in Compound 1a:

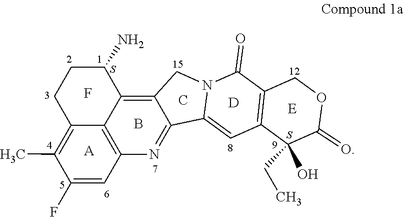

Exatecan can be coupled to a linker via the nitrogen atom of the amino group at position 1, such that the exatecan moiety, when bound to a linker or present within a linker-exatecan molecule (an (X—Z) molecule), for example as conjugated to an antibody, exatecan will have the structure of Compound 1b:

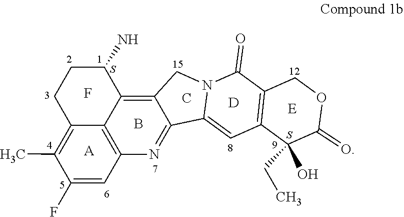

Thus, it will be understood that when exatecan of Compound 1a is attached to a linker (and, for example, when the linker is in turn attached to the antibody) via the amine at position 1, exatecan will be understood to be modified group at position 1 (i.e. the NH2 group at position 1 is replaced by an NH, or alternatively an OH or O group). For example, exatecan can be coupled to the antibody via a linker comprising a cleavable oligopeptide. Examples include di-, tri-, tetra- and penta-peptides such as the glycine and phenylalanine-containing peptides shown in U.S. Pat. No. 6,835,807, or the dipeptides valine-citrulline or valine-alanine attached to a PAB molecule, or the disclosures of which are incorporated herein by reference. Various suitable linker-Z structures are known that can liberate active exatecan or exatecan derivatives at the amino at position 1. Exatecan can be linked to a cleavable oligopeptide via a p-aminobenzyloxycarbonyl group (PAB) group attached to the position 1 amine, as shown in Formulae VII, VIII and IX, or in the (PEG (8U)-Val-Ala-PAB-Exatecan) linker of Example 7, which upon cleavage results in the liberation of the exatecan having the structure of Compound 1a. In one example, exatecan can be linked to a cleavable oligopeptide via a (CH₂—C(═O)) group attached to the position 1 amine, as shown in Formula III and Compound 13 (Dxd) as well as in the ggfg-Dxd linker of Example 7, which upon cleavage results in the liberation of the exatecan-containing Compound 13 (Dxd). Examples of substituents at the NH or NH2 of position 1 of exatecan of Compound 1 include —C(═O)—, —O—C(═O)—, or a (CH₂—C(═O)) comprising group such as —O—CH₂—C(═O)—, HO—O—CH₂—C(═O)—, —CH₂CH₂—C(═O)—, —CH₂CH₂CH₂—C(═O)—, —CH₂—O—CH₂—C(═O)— and —CH₂CH₂—O—CH₂—C(═O)—.

In one embodiment, a substituted exatecan derivative (e.g. derived at position 1) has the structure of compound 13.

The structure of Dxd is shown below:

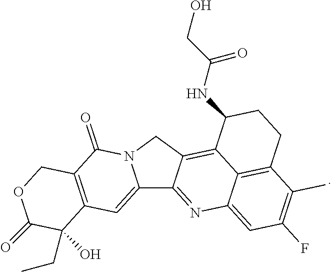 [SMILES: CC[C@@]1(O)C(=O)OCc2c1cc1n(c2=O)Cc2c-1nc1cc(F)c(C)c3c1c2[C@@H](NC(=O)CO)CC3]

Dxd can be coupled to a linker via the oxygen atom at the terminus/of the carbon chain, such that the Dxd moiety, when bound to a linker or present within a linker-Dxd molecule (an (X—Z) molecule), for example as conjugated to an antibody, the carbon-chain-terminal OH will be replaced by an O.

In one embodiment, the linker moiety (X—Z) is or comprises the structure shown in Formula VII, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues). An anti-Nectin-4 binding protein functionalized with a linker comprising the structure of Formula VII or Compounds 3 or 4 will release or yield (e.g. intracellularly, in the presence of Nectin-4 expressing tumor cells) a compound having the structure of Compound 1a.

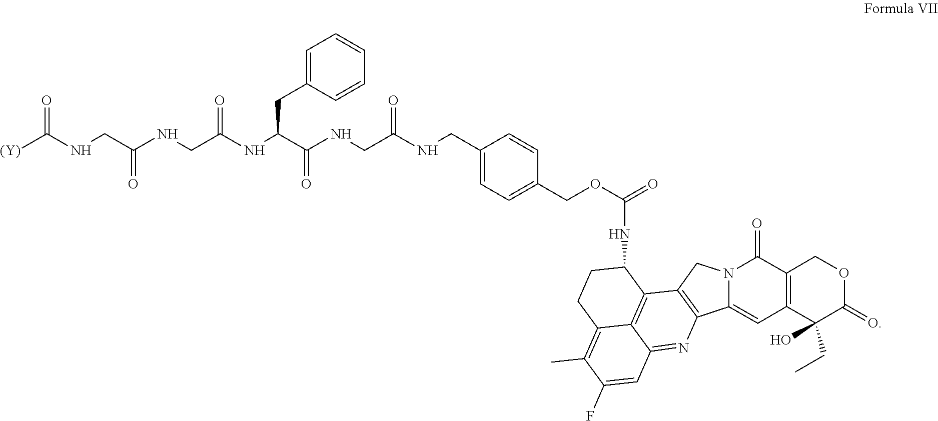

An exemplary linker having a maleimide as R group can have the structure of Compound 3, below. Such linker can be conjugated to an antibody via cysteine residues in the antibody after the interchain disulfide bounds are reduced with a reducing agent, e.g. tris(2-carboxyethyl) phosphine hydrochloride.

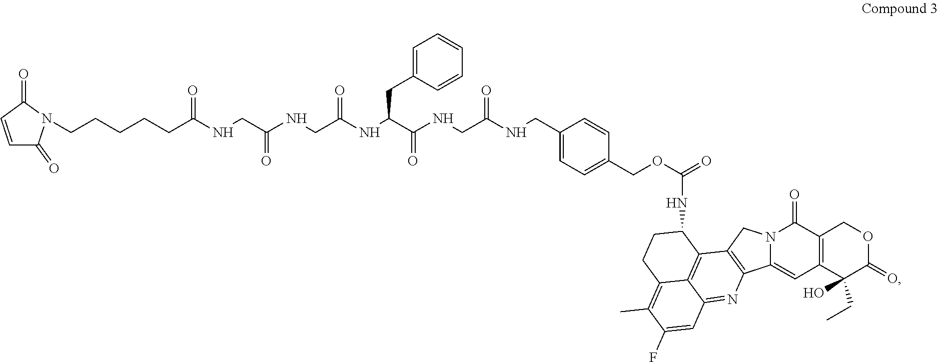 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)CNC(=O)[C@H](Cc2ccccc2)NC(=O)CNC(=O)CNC(=O)CCCCCN2C(=O)C=CC2=O)cc1)CC3]

The resulting antibody-drug conjugate will comprise an antibody comprising one or a plurality of cysteine residues functionalized with a compound having the structure of Compound 3 (wherein Compound 3 is bound via the S atom of the cysteine residue).

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X—Z), or an antibody functionalized therewith, has or comprises the structure shown as Compound 4, below:

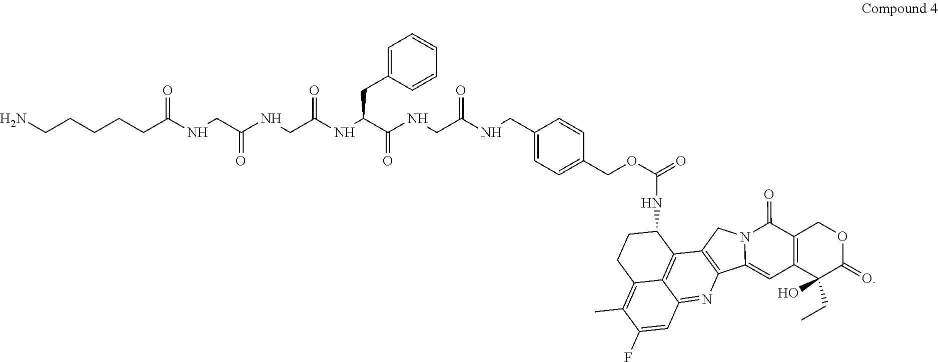 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)CNC(=O)[C@H](Cc2ccccc2)NC(=O)CNC(=O)CNC(=O)CCCCCN)cc1)CC3]

In one embodiment, the linker moiety (X—Z) is or comprises the structure shown in Formula VIII, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody). An anti-Nectin-4 binding protein functionalized with a linker comprising the structure of Formula VIII or Compounds 5, 6 or 7 will release or yield (e.g. intracellularly, in the presence of Nectin-4 expressing tumor cells) a compound having the structure of Compound 1a.

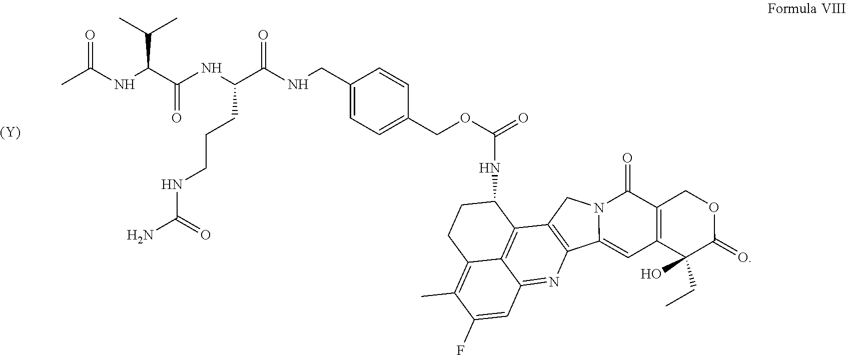 [SMILES: "(Y)" ; CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(C)=O)C(C)C)cc1)CC3]

An exemplary linker having a maleimide as R group can have the structure of Compounds 5 or 6, below. Such linker can be conjugated to an antibody via cysteine residues in the antibody after the interchain disulfide bounds are reduced with a reducing agent.

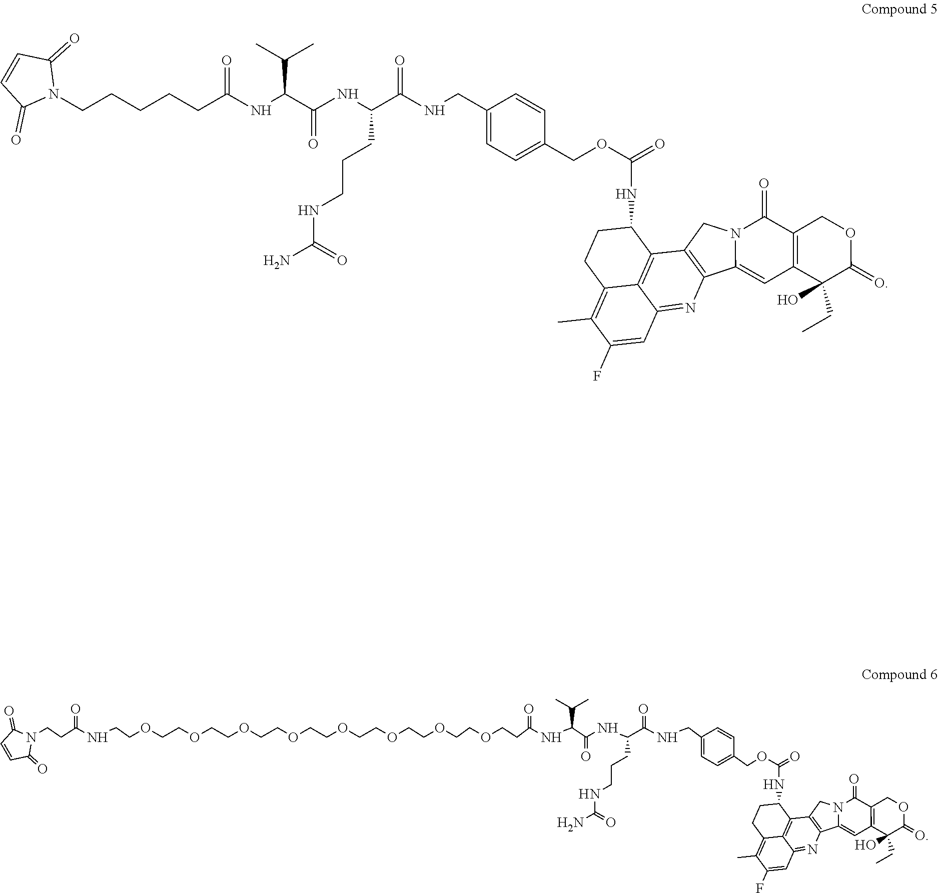 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(=O)CCCCCN2C(=O)C=CC2=O)C(C)C)cc1)CC3 ; CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(=O)CCOCCOCCOCCOCCOCCOCCOCCOCCNC(=O)CCN2C(=O)C=CC2=O)C(C)C)cc1)CC3]

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X—Z), or an antibody functionalized therewith, has or comprises the structure shown as Compound 7, below:

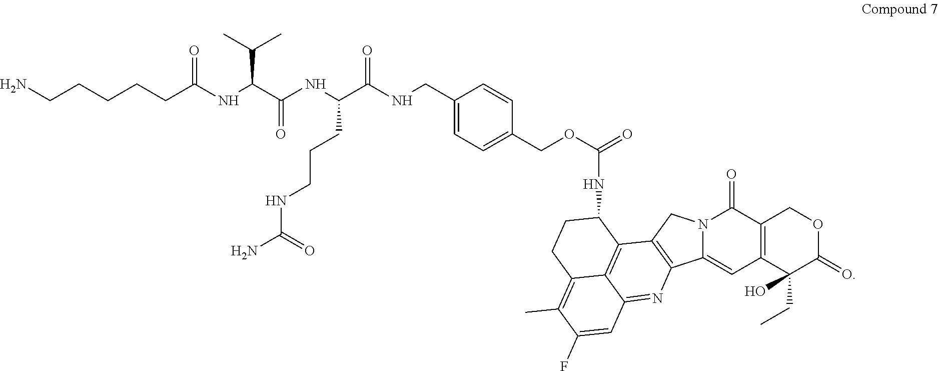 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(=O)CCCCCN)C(C)C)cc1)CC3]

In one embodiment, the linker moiety (X—Z) is or comprises the structure shown in Formula IX, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody). An anti-Nectin-4 binding protein functionalized with a linker comprising the structure of Formula IX will release (e.g. intracellularly, in the presence of Nectin-4 expressing tumor cells) a compound having the structure of Compound Ia.

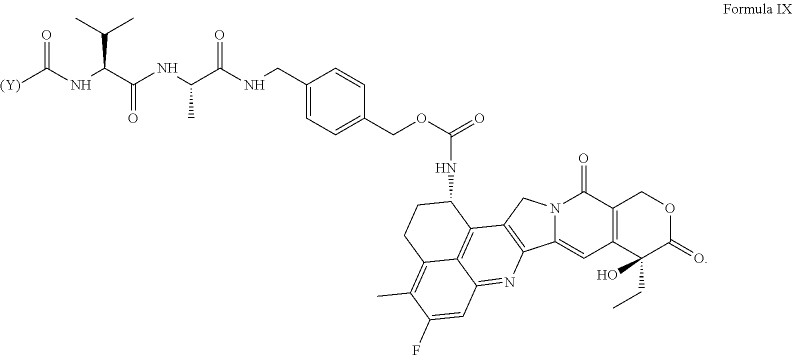

An exemplary linker having a maleimide as R group can have the structure of Compound 8, below. Such linker can be conjugated to an antibody via cysteine residues in the antibody after the interchain disulfide bounds are reduced with a reducing agent.

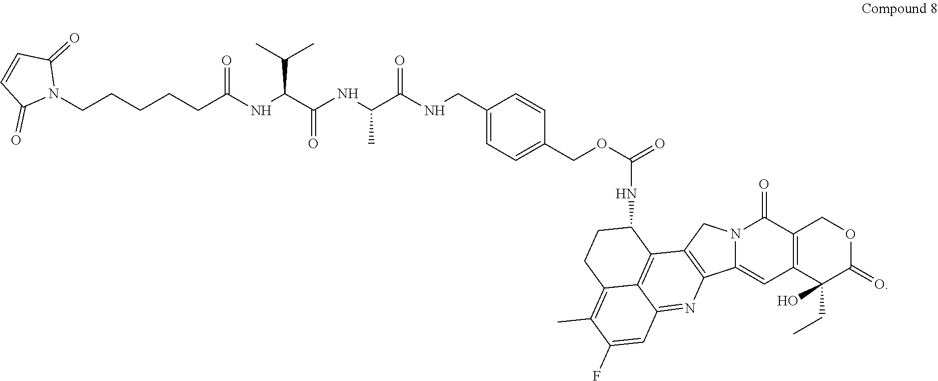 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](C)NC(=O)[C@@H](NC(=O)CCCCCN2C(=O)C=CC2=O)C(C)C)cc1)CC3]

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X—Z), or an antibody functionalized therewith, has or comprises the structure shown as Compound 9, below:

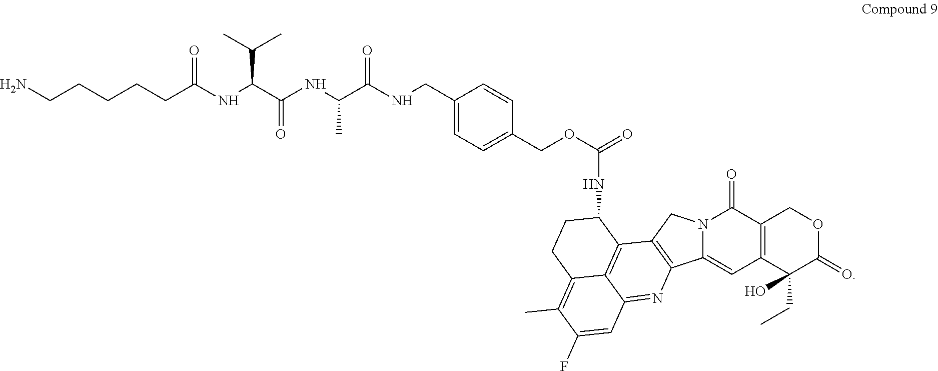 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](C)NC(=O)[C@@H](NC(=O)CCCCCN)C(C)C)cc1)CC3]

In one embodiment, the linker moiety (X—Z) is or comprises the structure shown in Formula X, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody).

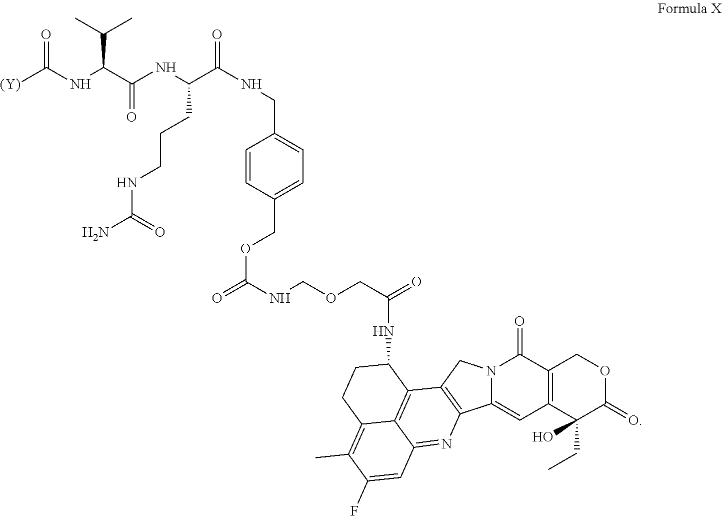

An exemplary linker having a maleimide as R group can have the structure of Compound 10, below. Such linker can be conjugated to an antibody via cysteine residues in the antibody after the interchain disulfide bounds are reduced with a reducing agent, e.g. tris (2-carboxyethyl) phosphine hydrochloride.

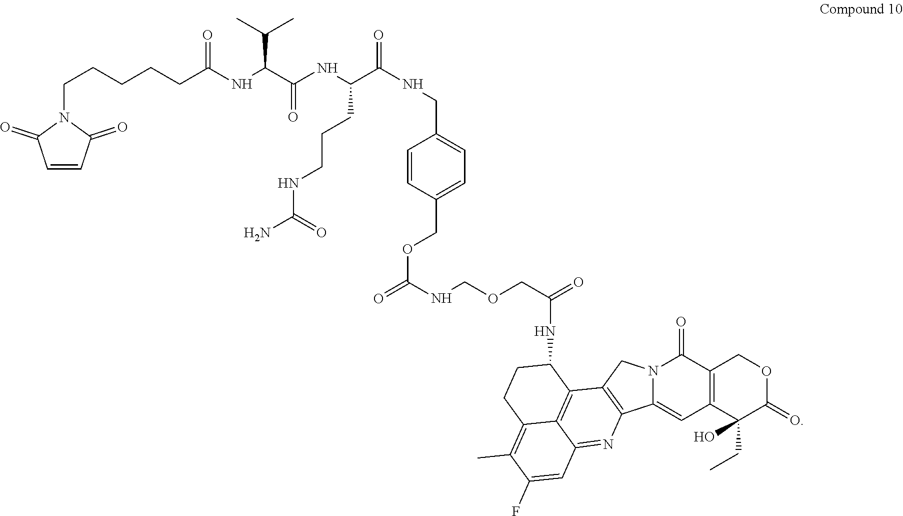 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(CNC(=O)[C@H](CCCNC(N)=O)NC(=O)[C@@H](NC(=O)CCCCCN2C(=O)C=CC2=O)C(C)C)cc1)CC3]

In one embodiment, the linker moiety (X—Z) is or comprises the structure shown in Formula XI, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody).

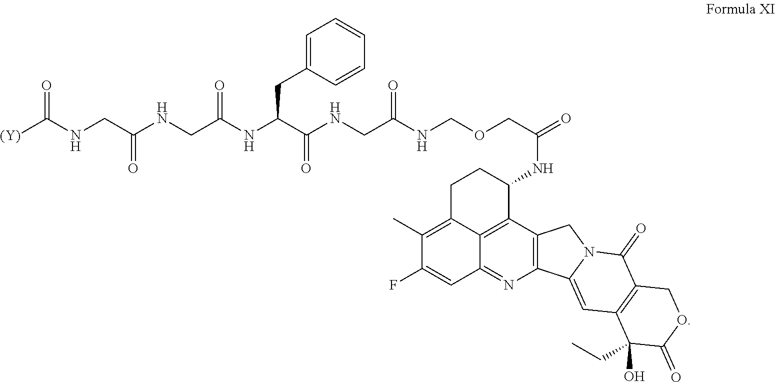

An exemplary linker having a maleimide as R group can have the structure of Compound 11, below. Such linker can be conjugated to an antibody via cysteine residues in the antibody after the interchain disulfide bounds are reduced with a reducing agent.

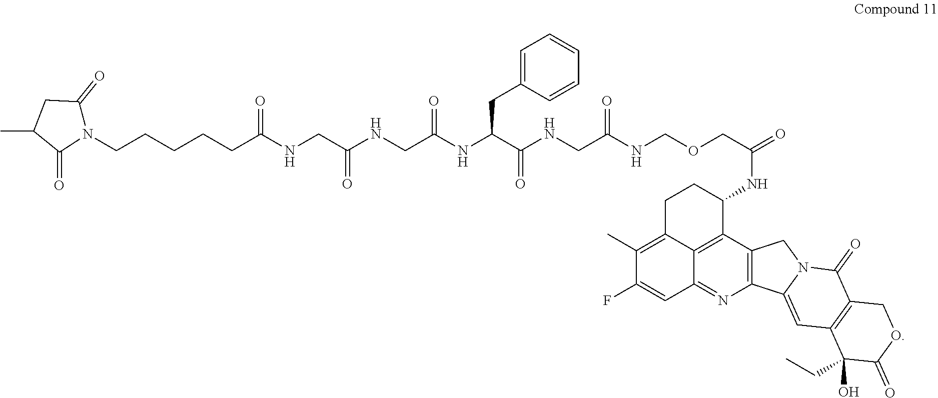 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X—Z), or an antibody functionalized therewith, has or comprises the structure shown as Compound 12, below:

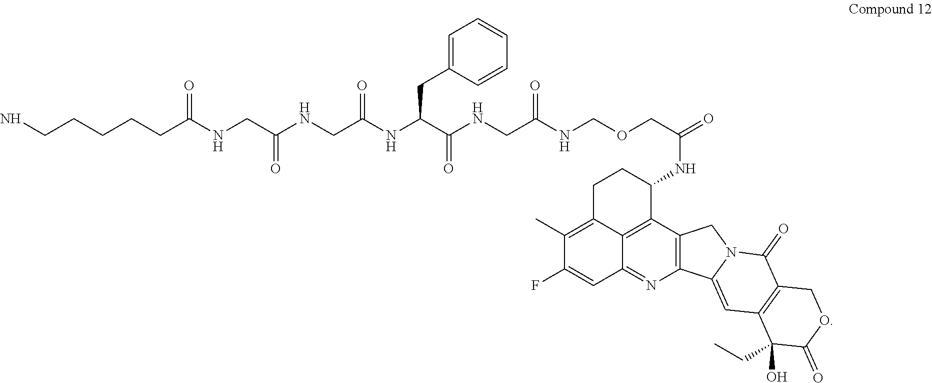 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN)CC3]

An anti-Nectin-4 binding protein functionalized with an oligopeptide-containing linker of Formulae XI or Compounds 11 and 12 result in the release (e.g. intracellularly, in the presence of Nectin-4 expressing tumor cells) of a substituted exatecan having a OH—CH2—C(═O) substituent present at position 1 amine, as shown in the structure below:

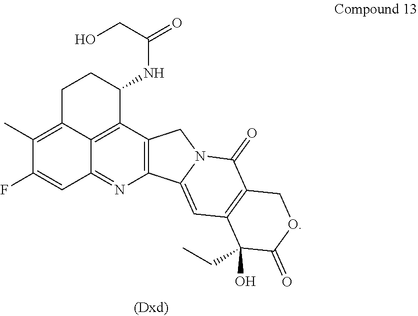 [SMILES: CC[C@@]1(O)C(=O)OCc2c1cc1n(c2=O)Cc2c-1nc1cc(F)c(C)c3c1c2[C@@H](NC(=O)CO)CC3]

The exemplary linkers of Formulae III, IV, V, VI, VII, VIII, IX, X or XI when prepared as a structure having a primary amine can reacted with antibody in the presence of a transglutamine enzyme (e.g. Bacterial Transglutaminase, BTG) such that the transglutaminase enzyme catalyzes the conjugation of the linker to an acceptor glutamine residue within the primary structure of the antibody, for example within an immunoglobulin constant domain or within a TGase recognition tag inserted or appended to (e.g., fused to) a constant region. Methods and linkers for use in BTG-mediated conjugation to antibodies is described in PCT publication no. WO2014/202773, the disclosure of which is incorporated by reference. Conjugation catalyzed by BTG permits precise control of the average drug: antibody ratio in a composition. The term “transglutaminase”, used interchangeably with “TGase” or “TG”, refers to an enzyme capable of cross-linking proteins through an acyl-transfer reaction between the γ-carboxamide group of peptide-bound glutamine and the E-amino group of a lysine or a structurally related primary amine such as amino pentyl group, e.g. a peptide-bound lysine, resulting in a Σ-(γ-glutamyl) lysine isopeptide bond. TGases include, inter alia, bacterial transglutaminase (BTG) such as the enzyme having EC reference EC 2.3.2.13 (protein-glutamine-γ-glutamyltransferase). The term “acceptor glutamine” residue, when referring to a glutamine residue of an antibody, means a glutamine residue that is recognized by a TGase and can be cross-linked by a TGase through a reaction between the glutamine and a lysine or a structurally related primary amine such as amino pentyl group. Preferably the acceptor glutamine residue is a surface-exposed glutamine residue. The term “TGase recognition tag” refers to a sequence of amino acids comprising an acceptor glutamine residue and that when incorporated into (e.g. appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the enzyme recognition tag. Examples of TGase recognition tags include the amino acid sequences: LLQ, LLQG, LSQG, GLLQ, SLLQG, GGGQGGL, LLQGG, LLQGA, LLQGG and LLQGA, or EQKLISEEDL or a variant having one or more (e.g., 2, 3, 4, 5, 6, 7, 8 or 9) sequence modifications.

As exemplified in WO2013/092983 and WO2020/188061, the disclosures of which are incorporate herein by reference, the linker-drug moiety (X—Z) can be conjugated to glutamine residues in an antibody (acceptor glutamines) in two-step process comprising a first step in which a moiety comprising a primary amine and a first reactive group (R) is conjugated to the antibody in the presence of BTG, followed by a step of reacting the antibody-linker conjugate with a molecule comprising (i) a second reactive group (R′) that is reactive with the first reactive group and (ii) the cytotoxic agent (Z). Examples of reactive group pairs R and R′ include a range of groups capable of biorthogonal reaction, for example 1,3-dipolar cycloaddition between azides and cyclooctynes (copper-free click chemistry), between nitrones and cyclooctynes, oxime/hydrazone formation from aldehydes and ketones and the tetrazine ligation (see also WO2013/092983). The resulting linker and functionalized antibody, or the Y element thereof, can thus comprise a RR′ group resulting from the reaction of R and R′, for example a triazole.

An anti-Nectin-4 immunoconjugate can be incorporated in a pharmaceutical formulation in a concentration from 1 mg/ml to 500 mg/ml, wherein said formulation has a pH from 2.0 to 10.0. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19th edition, 1995.

It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Administration of pharmaceutical compositions according to the invention may be through several routes of administration, for example, intravenous. Suitable antibody formulations can also be determined by examining experiences with other already developed therapeutic ADCs.

In any embodiment, a composition can be characterized as comprising a plurality of Nectin-4 binding immunoconjugates of the disclosure, wherein at least 70%, 80%, 90%, 95%, 98% or 99% of the immunoconjugates in a sample have at least 4, 6 or 8 amino acid residues per antibody that are functionalized with a linker disclosed herein. In any embodiment, a composition can be characterized as comprising a plurality of Nectin-4 binding immunoconjugates of the disclosure, wherein at least 70%, 80%, 90%, 95%, 98% or 99% of the immunoconjugates in a sample have at least 2, 4, 6 or 8 amino acid residues per antibody that are functionalized with the linker-camptothecin moiety, e.g., the (X—Z) unit or the (—(Y)-(Pep)-(Y′)—(Z)) unit of the formulae herein. In any embodiment, a composition can be characterized as comprising a plurality of Nectin-4 binding immunoconjugates of the disclosure, wherein at least 70%, 80%, 90%, 95%, 98% or 99% of the immunoconjugates in a sample have the same number of functionalized amino acid per antibody, optionally wherein the number is 4, 6 or 8.

### Diagnostics, Prognostics, and Treatment of Malignancies

In some aspects, described are methods as well as antibodies, antibody fragments and immunoconjugates useful in the diagnosis, prognosis, monitoring and treatment of a cancer characterized by tumor cells that express at their surface Nectin-4. In therapeutic methods, the treatment comprises administering to a human subject or individual an antibody of the disclosure. In some embodiments, provided are improved methods for improved delivery of cytotoxic agents to Nectin-4-positive tumors, particular camptothecin analogues. The improved delivery of the cytotoxic agent may be the result of antibody-mediated inhibition of cluster formation and/or anchorage-independent growth of Nectin-4 expressing tumor cells, resulting in improved tumor penetration of the cytotoxic agent and/or an ADC comprising such agent. Treatment with an ADC of the disclosure is particularly advantageous for the treatment of disease with lower or heterogeneous Nectin-4 expression, for patients having particularly resistant disease or for whom other ADCs are not suitable, and/or for use in combination with additional therapeutic agents that mediate toxicity as single agents (e.g., chemotherapeutic agents).

In one embodiment, an anti-Nectin-4 antibody or antibody fragment may be useful to sensitize a tumor to a cytotoxic or chemotherapeutic agent. The anti-Nectin-4 antibody may be useful in the reinforcement or enhancement of toxicity of an anticancer agent against cancer, as compared to the anticancer agent itself, by reducing the chemoresistance of cancerous cells. A cancer-sensitizing composition comprising anti-Nectin-4 antibody or antibody fragment may be useful to sensitize a tumor (e.g. a Pgp-expressing tumor) to an anticancer agent, to reduce the chemoresistance and to improve the therapeutic effect of the anticancer agent.

In one embodiment, an anti-Nectin-4 antibody or antibody fragment of the disclosure can be specified as being for use in treating a tumor in a human individual in combination with a chemotherapeutic agent, wherein the anti-Nectin-4 antibody, antibody fragment and the chemotherapeutic agent are formulated for separate administration and are administered concurrently or sequentially. For example, the treatment can comprising administering to the individual an effective amount of each of: (a) an anti-Nectin-4 antibody or antibody fragment of the disclosure, and (b) an anticancer agent, optionally a chemotherapeutic agent, optionally a chemotherapeutic agent known to be capable of being transported by P-glycoprotein (Pgp), optionally an anthracycline, a vinca alkaloid, an etoposide, a taxane, a platinum compound or optionally a camptothecin.

In one embodiment, the antibody or antibody fragment is conjugated to a cytotoxic agent, e.g., a camptothecin, an exatecan or SN-38 molecule. The antibody or antibody fragment will typically be conjugated to a plurality of molecules of a cytotoxic agent, e.g. a camptothecin analogue, an exatecan. A cytotoxic agent, for example a camptothecin or exatecan, can be conjugated to the antibody via a linker comprising a protease-cleavable oligopeptide linker. An exemplary pharmaceutical composition can comprise on average from 1 to 8 cytotoxic agent (e.g. camptothecin derivative, exatecan) molecules per antibody molecule, optionally from 2-8, from 4-8, from 6-8 cytotoxic agent molecules per antibody molecule, or for example about 2, 4, 5, 6, 7 or 8 cytotoxic agent molecules per antibody molecule. When the antibody or antibody fragment is conjugated to exatecan (i.e. via a linker), such immunoconjugate is particularly advantageous to treat a Pgp-expressing tumor (e.g. a tumor characterized by expression of Pgp (MDR1). Tumor characterized by expression of Pgp include tumors following treatment with chemotherapeutic agents and ADCs, for example it has been shown that treatment with ADCs comprising auristatin (MMAE) payloads induce Pgp expression on tumors. Additionally, a range of tumors characterized by natural Pgp/MDR1 expression are known, for example significant levels of MDR1 are expressed by kidney cancer, adrenocortical carcinoma, cholangiocarcinoma, liver cancer, rectal cancer, colon cancer, brain cancer, pancreatic cancer, prostate cancer, mesothelioma, stomach cancer, AML, thyroid cancer, DLBC, esophageal cancer, breast cancer, sarcoma, testical cancer, uterine cancer, thymoma, cervical cancer, lung cancer, bladder cancer (e.g., urothelial carcinoma), and head and neck squamous cell carcinoma.

The Nectin-4 binding agent (e.g. anti-Nectin-4 antibody or antibody fragment) conjugated to a cytotoxic agent can be used advantageously to treat an individual having a Nectin-4-expressing cancer characterized by tumor cells that express Nectin-4 (e.g. at the tumor cell membrane or cell surface). Example of such cancers are urothelial cancer, breast cancer (e.g. triple-negative breast cancer; HER2-positive breast cancer), non-small cell lung cancer, pancreatic cancer, ovarian cancer, gastric cancer, colorectal cancer (e.g. colon cancer), head and neck squamous cell carcinoma and esophageal cancer.

The Nectin-4 binding agent conjugated to a cytotoxic agent can be used in Nectin-4 high-expressing tumors.

The Nectin-4 binding agent conjugated to a cytotoxic agent can also be used in heterogeneous and/or low Nectin-4-expressing tumors. In such tumors, the immunoconjugates of the disclosure can provide advantageous efficacy, optionally via avoidance of MDR1-mediated resistance and/or bystander anti-tumor effects.

The Nectin-4 binding agent conjugated to a cytotoxic agent can be used advantageously to treat an individual regardless of Nectin-4 expression levels, regardless of heterogeneity of Nectin-4 expression levels on tumor cells within an individual, and/or regardless of whether or not the individual has been previously treated with enfortumab verdotin. Where the cytotoxic agent is an exatecan, the Nectin-4 binding agent conjugated to a cytotoxic agent can also be used advantageously to treat an individual regardless of tumor Pgp expression and/or regardless of whether or not the individual has been previously treated with an ADC comprising a cytotoxic agent that is capable of being transported by Pgp (for example an ADC comprising an anti-Nectin-4 antibody conjugated to a camptothecin analogue other than exatecan, an ADC comprising an anti-Nectin-4 antibody conjugated to Dxd (e.g. via a GGFG-containing linker), or an anti-Nectin-4 antibody conjugated to an auristatin, an anthracycline, a vinca alkaloid, an etoposide, a taxane or a platinum compound).

In one embodiment, the Nectin-4 binding agent conjugated to a camptothecin derivative molecule can be used advantageously to treat an individual who has been previously treated with enfortumab verdotin. Such an individual may optionally have a cancer characterized by heterogeneous and/or low Nectin4-expressing tumors following enfortumab verdotin treatment. Such an individual may optionally have a cancer characterized by Pgp-expressing tumors following enfortumab verdotin treatment. An individual may have a cancer that is resistant, has not responded, has relapsed and/or progressed despite (e.g. during or following) treatment with an antibody conjugated to an auristatin or MMAE molecule (e.g., enfortumab vedotin). For example, the individual may have a locally advanced or metastatic urothelial cancer and has previously received treatment with an antibody conjugated to an auristatin or MMAE molecule (e.g., enfortumab vedotin).

In one embodiment, an immunoconjugate comprising a Nectin-4 binding agent conjugated via a linker to an exatecan is used advantageously to treat an individual who has been previously treated with an immunoconjugate comprising a Nectin-4 binding agent conjugated via a linker to a cytotoxic agent that is capable of being transported by Pgp. In one embodiment, an immunoconjugate comprising an Nectin-4 binding agent conjugated via a linker to an exatecan is used advantageously to treat an individual having a cancer characterized by heterogeneous and/or low Nectin4-expressing tumors following treatment with an immunoconjugate comprising a Nectin-4 binding agent conjugated via a linker to a cytotoxic agent that is capable of being transported by Pgp. In one embodiment, an immunoconjugate comprising a Nectin-4 binding agent conjugated via a linker to an exatecan is used advantageously to treat an individual having a cancer that is resistant, has not responded, has relapsed and/or progressed despite (e.g. during or following) treatment with an Nectin-4 binding agent conjugated via a linker to an cytotoxic agent that is capable of being transported by Pgp. Optionally, the Nectin-4 binding agent conjugated via a linker to a cytotoxic agent that is capable of being transported by Pgp comprises an anti-Nectin-4 antibody conjugated via a linker to a camptothecin analogue so as to release, upon cleavage of the linker, a camptothecin analogue other than exatecan (e.g. a Dxd or deruxtecan). Optionally, the Nectin-4 binding agent conjugated via a linker to a cytotoxic agent that is capable of being transported by Pgp comprises an anti-Nectin-4 antibody conjugated to Dxd (e.g. via a GGFG-containing linker). Optionally, the Nectin-4 binding agent conjugated via a linker to a cytotoxic agent that is capable of being transported by Pgp comprises an anti-Nectin-4 antibody conjugated to an auristatin, an anthracycline, a vinca alkaloid, an etoposide, a taxane or a platinum compound). Optionally, the Nectin-4 binding agent conjugated via a linker to a cytotoxic agent that is capable of being transported by Pgp comprises an anti-Nectin-4 antibody that binds to the IgV domain of Nectin-4. Optionally, the Nectin-4 binding agent conjugated via a linker to a cytotoxic agent that is capable of being transported by Pgp comprises an anti-Nectin-4 antibody that binds to the VC1 bridging domain of Nectin-4. In one embodiment, the immunoconjugate comprising an Nectin-4 binding agent conjugated via a linker to an exatecan comprises a linker having an enzymatically cleavable moiety (and optionally a self-immolating spacer) that results in the release of exatecan upon cleavage.

In advanced recurrent or metastatic urothelial cancer, a significant proportion of individuals will express high levels of Nectin-4 on tumor cells, e.g. H-score of at least 290 (See EV-201 clinical trial Cohort 1 Nectin-4 expression). However, a subset of patients have an H-score of less than 250, and some less than 200. A minority of patient had an H-score of less than 150, with some having an H-score of less than 100. In triple negative breast cancer (TNBC), it has been reported that 62% of patients have high Nectin-4 expression on tumor cells and 38% have low Nectin-4 expression on tumor cells (Rabat et al., 2017 Annals Onc. 28:769-776). In other cancer types, median H-score values for Nectin-4 expression have typically been lower than that observed in UC, notably in non-small cell lung cancer, pancreatic cancer, ovarian cancer, head and neck squamous cell carcinoma and esophageal cancer.

In one embodiment, an individual treated according to the disclosure has an advanced recurrent or metastatic cancer, optionally an advanced recurrent or metastatic urothelial cancer.

In one embodiment, an individual treated according to the disclosure has a cancer (e.g., a breast cancer) that tests positive for estrogen receptors and/or progesterone receptors, and tests negative for epidermal growth factor receptor 2 (HER2) or excess HER2 protein, optionally the cancer test positive for HER2 but HER2 is expressed at low levels.

In one embodiment, an individual treated according to the disclosure has a triple-negative breast cancer (TNBC), e.g., a breast cancer that tests negative for estrogen receptors, progesterone receptors, and excess HER2 protein.

In one embodiment, an individual treated according to the disclosure has a cancer (e.g. a breast cancer) that tests positive for HER2 protein, optionally wherein the cancer is expresses excess HER2 protein (HER2 over-expression), optionally the cancer is expresses low levels of HER2 protein (lower than excess HER2 expression). In one embodiment, the individual is treated with an anti-Nectin-4 ADC, in combination with an agent (e.g. antibody) that binds a HER2 polypeptide (e.g. trastuzumab, pertuzumab); optionally wherein the agent that binds HER2 is an ADC; optionally wherein the antibody that binds HER2 is conjugated to a cytotoxic agent, optionally an auristatin, a maytansinoid (e.g. DM1) or a camptothecin analogue (e.g. Compound 1, 2 or 13); optionally wherein the antibody that binds HER2 is trastuzumab emtansine or trastuzumab deruxtecan (DS-8201a; Enhertu™).

In one embodiment, an individual treated according to the disclosure has a non-small cell lung cancer, optionally a lung adenocarcinoma.

In one embodiment, an individual treated according to the disclosure has a pancreatic cancer.

In one embodiment, an individual treated according to the disclosure has an ovarian cancer.

In one embodiment, an individual treated according to the disclosure has a head and neck squamous cell carcinoma.

In one embodiment, an individual treated according to the disclosure has an oesophageal cancer.

In one embodiment, an individual treated according to the disclosure has a colorectal cancer. Colorectal cancer (CRC) as used herein refers to colon cancer, rectal cancer, and colorectal cancer (cancer of both the colon and rectal areas).

In one embodiment, an individual treated according to the disclosure has a NSCLC or lung adenocarcinoma, a gastric cancer, a colorectal carcinoma, a pancreatic cancer, a urothelial carcinoma or bladder cancer that tests positive for HER2 protein, optionally wherein the cancer expresses excess HER2 protein (HER2 over-expression), optionally the cancer is expresses low levels of HER2 protein (lower than excess HER2 expression). In one embodiment, the individual is treated with an anti-Nectin-4 ADC according to disclosure, in combination with an agent (e.g. antibody) that binds HER2 polypeptides (e.g. an antibody comprising the heavy and light chains CDRs or variable regions of trastuzumab or pertuzumab); optionally wherein the agent that binds HER2 is an ADC; optionally wherein the antibody that binds HER2 is conjugated to a cytotoxic agent, optionally an auristatin, a maytansinoid (e.g. DM1) or a camptothecin analogue (e.g. Compound 1, 2 or 13); optionally wherein the antibody that binds Her2 is trastuzumab emtansine or trastuzumab deruxtecan (DS-8201a).

As shown herein, an ADC that releases exatecan upon cleavage (e.g. upon cleavage of the intracellularly cleavable linker that comprises the cytotoxic agent Z) is particularly advantageous in treatment of tumors that are resistant to ADCs with linkers that upon cleavage release cytotoxic agents (other than exatecan) that are transported by Pgp, for example auristatins or Dxd. Accordingly, in one embodiment, where the HER2 positive cancer is treated with a combination of anti-Nectin-4 binding agent (e.g. anti-Nectin-4 ADC) and anti-HER2 ADC, the anti-HER2 ADC can be designed to release exatecan upon cleavage (e.g. upon cleavage of the intracellularly cleavable linker that comprises the cytotoxic agent Z). The anti-Nectin-4 binding agent (e.g. anti-Nectin-4 ADC) can be designed to release exatecan upon cleavage (e.g. upon cleavage of the intracellularly cleavable linker that comprises the cytotoxic agent Z, or can release any other cytotoxic agent (Z), for example where Z is a taxane, anthracycline, camptothecin, epothilone, mytomycin, combretastatin, vinca alkaloid, nitrogen mustard, maytansinoid, duocarmycin, tubulysin, dolastatin, auristatin, enediyne, pyrrolobenzodiazepine, amatoxin or ethylenimine.

Accordingly, in one aspect, the present disclosure provides an immunoconjugate that binds a human Nectin-4 polypeptide, for use in the treatment of cancer (e.g., a HER2 positive Nectin-4-positive cancer), wherein the immunoconjugate that binds a human Nectin-4 polypeptide is represented by the formula:

Ab_{N4}-(X_{N4}-(Z_{N4}))
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent;
wherein the immunoconjugate that binds a human Nectin-4 polypeptide is for use in combination with an immunoconjugate that binds a human HER2 polypeptide represented by the formula:

Ab_{HER2}-(X_{HER2}-(Z_{HER2}))
wherein,
Ab_{HER2} is a polypeptide, peptide or antibody that specifically binds to a human HER2 polypeptide, optionally wherein the Ab HER2 comprises the heavy and light chain CDRs or variable regions of trastuzumab or pertuzumab;
X_{HER2} is a molecule which connects Ab_{HER2} and Z_{HER2}, wherein X_{HER2} comprises a cleavable moiety, e.g., under physiological conditions, optionally under intracellular conditions, optionally a protease-cleavable di-, tri-, tetra- or penta-peptide; and
Z_{HER2} is an exatecan. The immunoconjugate that binds a human HER2 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

Also, in one aspect, the present disclosure provides an immunoconjugate that binds a human HER2 polypeptide, for use in the treatment of cancer e.g., a HER2 positive Nectin-4-positive cancer), wherein the immunoconjugate that binds a human HER2 polypeptide is represented by the formula:

Ab_{HER2}-(X_{HER2}-(Z_{HER2}))
wherein
Ab_{HER2} is a polypeptide, peptide or antibody that specifically binds to a human HER2 polypeptide, optionally wherein the Ab HER2 comprises the heavy and light chain CDRs or variable regions of trastuzumab or pertuzumab;
X_{HER2} is a molecule which connects Ab_{HER2} and Z_{HER2}, wherein X_{HER2} comprises a cleavable moiety, e.g., under physiological conditions, optionally under intracellular conditions, optionally a protease-cleavable di-, tri-, tetra- or penta-peptide; and
Z_{HER2} is an exatecan;
wherein the immunoconjugate that binds a human HER2 polypeptide is for use in combination with an immunoconjugate that binds a human Nectin-4 polypeptide represented by the formula:

Ab_{N4}-(X_{N4}—(Z_{N4}))
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent. The immunoconjugate that binds a human HER2 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

In one aspect, the treatment methods of the disclosure are independent of the assessment or detection of Nectin-4 expression in tumor tissue and/or independent of the expression level of Nectin-4 on tumor cells and/or the frequency or number of Nectin-4-expressing tumor cells in a tissue sample from said individual. In one aspect, the treatment methods of the disclosure are independent of the assessment or detection of tumor Pgp (MDR1) expression.

In one aspect, the present invention provides methods of treating a cancer and/or eliciting an anti-tumor immune response in an individual in need thereof, wherein said individual has advanced recurrent or metastatic urothelial cancer or breast cancer (e.g. TNBC), wherein said methods do not necessitate the pre-determination of whether the individual has tumor tissue comprising cells (e.g. tumor cells) that express Nectin-4 or not.

In one aspect, the present invention provides methods of treating a cancer and/or killing tumor cells in an individual in need thereof, wherein said individual has advanced recurrent or metastatic urothelial cancer or breast cancer (e.g. TNBC), wherein said methods do not necessitate the pre-determination of whether or not the individual has tumor tissue comprising cells (e.g. tumor cells) that express high-levels of Nectin-4, e.g. as defined by an immunohistochemistry assessment (e.g. an H-score or other appropriate IHC scoring method).

In one aspect, the methods of treating a cancer and/or killing tumor cells in an individual do not necessitate the pre-determination of the level of Nectin-4 expression of tumor cells.

In one aspect, the methods of treating a cancer in an individual, optionally an advanced recurrent or metastatic urothelial cancer or a breast cancer (e.g. TNBC, HER2 positive cancer), comprise treating an individual having a cancer characterized by an H-score for Nectin-4 expression of no more than, or less than, 290, 250, 200, 150 or 100.

In any embodiment for treating or preventing a cancer in an individual, the method can be specified as comprising the steps of: (i) identifying an individual whose tumor cells express Nectin-4 (e.g. as determined by immunohistochemistry), and (ii) administering to the individual an effective amount of an anti-Nectin-4 antibody drug conjugate of the disclosure.

In any embodiment for treating or preventing a cancer in an individual, the method can be specified as comprising the steps of (i) identifying an individual whose tumor cells express (a) Nectin-4 (e.g. as determined by immunohistochemistry), and (b) HER2, optionally wherein the tumor cells express low levels of HER2 (e.g. as determined by immunohistochemistry; as determined by Herceptest™) and (ii) administering to the individual an effective amount of an anti-Nectin-4 antibody drug conjugate of the disclosure, optionally in combination with an agent (e.g. antibody) that binds Her2 polypeptides (e.g. trastuzumab, pertuzumab); optionally wherein the antibody that binds Her2 is an ADC; optionally wherein the antibody that binds Her2 is conjugated to a cytotoxic agent, optionally an auristatin, a maytansinoid (e.g. DM1) or a camptothecin derivative (e.g. Compound 1, 2 or 13); optionally wherein the antibody that binds Her2 is trastuzumab emtansine or trastuzumab deruxtecan (DS-8201a).

In any embodiment for treating or preventing a cancer in an individual, the method can be specified as comprising the steps of: (i) identifying an individual whose tumor cells have a low or moderate level of Nectin-4 expression (e.g. as determined by immunohistochemistry), and (ii) administering, to the individual identified in step (i), an effective amount of an anti-Nectin-4 antibody drug conjugate of the disclosure.

In any embodiment for treating or preventing a cancer (e.g. a Nectin-4 positive cancer) in an individual, the method can be specified as comprising the steps of: (i) identifying an individual whose cancer is characterized by a low level of Nectin-4 expression (e.g. as determined by immunohistochemistry), and (ii) administering, to the individual identified in step (i), an effective amount of an anti-Nectin-4 antibody drug conjugate of the disclosure. In one embodiment, the individual has a cancer characterized by an H-score for Nectin-4 expression of no more than, or less than, 150 or 100.

In any embodiment for treating or preventing a cancer (e.g. a Nectin-4 positive cancer) in an individual, the method can be specified as comprising the steps of: (i) identifying an individual whose cancer is characterized by a moderate level of tumor Nectin-4 expression (e.g. as determined by immunohistochemistry), and (ii) administering to the individual an effective amount of an anti-Nectin-4 antibody drug conjugate of the disclosure. In one embodiment, the individual has a cancer characterized by an H-score by an H-score for Nectin-4 expression of no more than, or less than, 290, 250, 200, 150, optionally further wherein the cancer is characterized by an H-score for Nectin-4 expression of at least 100.

In a still further embodiment, provided is a method for treating or preventing a cancer (e.g. a Nectin-4 positive cancer) in an individual comprising: (i) identifying an individual whose cancer is characterized by an H-score for tumor Nectin-4 expression of no more than, or less than, 290, 250, 200, 150, 120 or 100, and (ii) administering to the individual an effective amount of an anti-Nectin-4 antibody drug conjugate of the disclosure. Optionally, step (i) may be specified as comprising a step of assessing Nectin-4 expression on tumor cells by histochemistry (e.g. IHC).

In a still further embodiment, provided is a method for treating or preventing a cancer (e.g. a Nectin-4 positive cancer; a breast cancer) in an individual comprising: (i) identifying an individual whose cancer is characterized by a QS-score for tumor Nectin-4 expression of no more than, or less than, 200, 150, 120 or 100, and (ii) administering to the individual an effective amount of an anti-Nectin-4 antibody drug conjugate of the disclosure. Optionally, step (i) may be specified as comprising a step of assessing Nectin-4 expression on tumor cells by histochemistry (e.g. IHC).

A biological sample from an individual, for example from a biopsy, can be obtained and assessed. Optionally, the sample is preserved as formaldehyde (e.g. formalin)-fixed paraffin embedded (FFPE) samples. Following deparaffination, the slides are amenable to methods to detect the expression of Nectin-4 (and/or HER2).

Expression of Nectin-4 and/or HER2 in tumor cells can be determined by any methods known in the art. In certain embodiments, assays include immunohistochemistry (IHC) assays, fluorescence activated cell sorting (FACS) assays, for example quantitative FACS, ELISA, immunoblotting (e.g. western blotting, dot blotting, or in-cell western blotting), and other immunoassays.

IHC staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, in some embodiments, polyclonal antisera, and in some embodiments, monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In some embodiments, the IHC assay is a direct assay, wherein binding of antibody to the target antigen is determined directly. This direct assay uses a labeled reagent, such as a fluorescent tag or an enzyme-labeled primary antibody, which can be visualized without further antibody interaction. In some embodiments, the IHC assay is an indirect assay. In a typical indirect assay, unconjugated primary antibody binds to the antigen and then a labeled secondary antibody binds to the primary antibody. Where the secondary antibody is conjugated to an enzymatic label, a chromagenic or fluorogenic substrate is added to provide visualization of the antigen. Signal amplification occurs because several secondary antibodies may react with different epitopes on the primary antibody. The primary and/or secondary antibody used for immunohistochemistry typically will be labeled with a detectable molecule. Numerous labels are available, including radioisotopes, colloidal gold particles, fluorescent labels, and enzyme-substrate labels.

Strongly staining, moderately staining, and weakly staining are descriptions well known to those in the art. In some aspects strongly staining, moderately staining, and weakly staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

Control cell lines (e.g., centrifuged into a pellet and formalin fixed and paraffin embedded, e.g., and prepared as a tissue microarray, and e.g., stained with anti-Nectin-4 antibodies) with various staining intensities (e.g., when stained with anti-Nectin-4 antibodies) may be utilized as controls for IHC analysis. One of ordinary skill understands that other control cell pellets with negative, weak, moderate and high c-met staining intensity may readily be identified using the teachings of the present application and methods well known in the art and disclosed herein.

In some embodiments, a cancer or tumor is considered to be a Nectin-4-expressing cancer tumor when it is (e.g. is determined to be using an IHC assay) Nectin-4 positive.

In some embodiments, an individual's cancer or tumor is Nectin-4 positive when 5% or more of the tumor cells in the sample express Nectin-4 protein (e.g., express Nectin-4 protein at any intensity). In some embodiments, an individual's cancer or tumor is Nectin-4 positive when 10% or more of the tumor cells in the sample express Nectin-4 protein (e.g., express Nectin-4 protein at any intensity). In some embodiments, an individual's cancer or tumor is Nectin-4 positive when 20% or more of the tumor cells in the sample express Nectin-4 protein (e.g., express Nectin-4 protein at any intensity). In some embodiments, an individual's cancer or tumor is Nectin-4 positive when 30% or more of the tumor cells in the sample express Nectin-4 protein (e.g., express Nectin-4 protein at any intensity). In some embodiments, an individual's cancer or tumor is Nectin-4 positive when 40% or more of the tumor cells in the sample express Nectin-4 protein (e.g., express Nectin-4 protein at any intensity). In some embodiments, an individual's cancer or tumor is Nectin-4 positive when 50% or more of the tumor cells in the sample express Nectin-4 protein (e.g., express Nectin-4 protein at any intensity). In some embodiments, an individual's cancer or tumor is Nectin-4 positive when 60% or more of the tumor cells in the sample express Nectin-4 protein (e.g., express Nectin-4 protein at any intensity). In some embodiments, an individual's cancer or tumor is Nectin-4 positive when 70% or more of the tumor cells in the sample express Nectin-4 protein (e.g., express Nectin-4 protein at any intensity). In some embodiments, an individual's cancer or tumor is Nectin-4 positive when 80% or more of the tumor cells in the sample express Nectin-4 protein (e.g., express Nectin-4 protein at any intensity). In some embodiments, an individual's cancer or tumor is Nectin-4 positive when 90% or more of the tumor cells in the sample express Nectin-4 protein (e.g., express Nectin-4 protein at any intensity).

In some embodiments, an individual's cancer or tumor is Nectin-4 positive when 5% or more of the tumor cells in the sample express Nectin-4 protein with a moderate and/or strong staining intensity. In some embodiments, an individual's cancer or tumor is Nectin-4 positive when 10% or more of the tumor cells in the sample express Nectin-4 protein with a moderate and/or strong staining intensity. In some embodiments, an individual's cancer or tumor is Nectin-4 positive when 20% or more of the tumor cells in the sample express Nectin-4 protein with a moderate and/or strong staining intensity. In some embodiments, an individual's cancer or tumor is Nectin-4 positive when 30% or more of the tumor cells in the sample express Nectin-4 protein with a moderate and/or strong staining intensity. In some embodiments, an individual's cancer or tumor is Nectin-4 positive when 40% or more of the tumor cells in the sample express Nectin-4 protein with a moderate and/or strong staining intensity. In some embodiments, an individual's cancer or tumor is Nectin-4 positive when 50% or more of the tumor cells in the sample express Nectin-4 protein with a moderate and/or strong staining intensity. In some embodiments, an individual's cancer or tumor is Nectin-4 positive when 60% or more of the tumor cells in the sample express Nectin-4 protein with a moderate and/or strong staining intensity. In some embodiments, an individual's cancer or tumor is Nectin-4 positive when 70% or more of the tumor cells in the sample express Nectin-4 protein with a moderate and/or strong staining intensity. In some embodiments, an individual's cancer or tumor is Nectin-4 positive when 80% or more of the tumor cells in the sample express Nectin-4 protein with a moderate and/or strong staining intensity. In some embodiments, an individual's cancer or tumor is Nectin-4 positive when 90% or more of the tumor cells in the sample express Nectin-4 protein with a moderate and/or strong staining intensity.

Assessing immunohistochemistry assays in order to determine whether an individual's cancer or tumor is characterized by high Nectin-4 expression or not (e.g. low or moderate Nectin-4 expression) will typically involve application of a known scoring method.

Low, moderate and high tumor Nectin-4 expression can be determined based on an “H-score” as described in US Pat. Pub. No. 2013/0005678. An H-score is obtained by the formula: (3×percentage of strongly staining cells)+ (2×percentage of moderately staining cells)+ (percentage of weakly staining cells), giving a range of 0 to 300. H-score has been used in particular in UC.

In some embodiments of any of the methods herein, low or moderate Nectin-4 expression (e.g., tumors or tumor cells having a low or moderate level of Nectin-4 expression) corresponds to an H-score of about 250 or lower, about 220 or lower, about 200 or lower, about 180 or lower, about 160 or lower, about 150 or lower, about 140 or lower, about 130 or lower, about 120 or lower, about 110 or lower or about 100 or lower.

In some embodiments of any of the methods herein, low Nectin-4 expression (e.g., tumors or tumor cells having a low level of Nectin-4 expression) corresponds to an H-score of 200 or lower, about 180 or lower, about 160 or lower, about 150 or lower, about 140 or lower, about 130 or lower, about 120 or lower, about 110 or lower or about 100 or lower.

In some embodiments of any of the methods herein, high Nectin-4 expression (e.g. tumors or tumor cells having a high level of Nectin-4 expression) corresponds to an H-score of about 290 or higher.

In another example, Nectin-4 staining can be scored according to the Quick score (QS) by using the following formula: QS=P (percentage of positive cells)×I (intensity), the maximum score being 300. QS has been used for example in breast cancer. For example, in TNBC some research groups have defined low Nectin-4 expression group as a QS=or <100. In some embodiments of any of the methods herein, low or moderate Nectin-4 expression (e.g., tumors or tumor cells having a low or moderate level of Nectin-4 expression) corresponds to QS-score of about 200 or lower, about 180 or lower, about 160 or lower, about 150 or lower, about 140 or lower, about 130 or lower, about 120 or lower, about 110 or lower or about 100 or lower.

Assays for assessing tumor cell expression of HER2 are well-known in the art. For example, assays such as the FDA-approved SPOT-Light HER2 CISH can be used to detect HER2 over-expression. Chromogenic in situ hybridization (CISH) detects HER2 gene amplification. This technique, also referred to as Subtraction Probe Technology Chromogenic

In Situ Hybridization, is a test used see if breast cancer cells overexpress HER2 receptor proteins at the cell surface.

Another widely used assay for HER2 is the HercepTest™ (Dako North America, Inc.), a semiquantitative immunohistochemical assay used to determine HER2 protein overexpression in in formalin-fixed, paraffin-embedded cancer tissue. For example, tumors expressing low levels of HER2 can be identified by a score of +1 to +2 via HercepTest™.

In one aspect, the treatment is used in an individual who has existing neuropathy, diabetes or hyperglycemia, cardiac insufficiency, an ocular pathology. Such conditions can render the individual unsuitable for treatment with anti-Nectin-4 ADCs such as enfortumab verdotin having higher toxicity or a more narrow therapeutic window than the anti-Nectin-4 antibody drug conjugates of the disclosure.

In any embodiment, the method of treatment may optionally comprise the steps of (a) assessing the cancer stage and/or disease progression in the individual; and (b) if the individual has recurrent, metastatic and/or progressing cancer, administering to the individual an effective amount of an anti-Nectin-4 antibody drug conjugate of the disclosure.

In some embodiments, the invention includes a method of treating a tumor in an individual having a urothelial cancer, comprising: (a) assessing the cancer stage and/or disease progression in the individual; and (b) if the individual has recurrent, metastatic and/or progressing cancer, administering to the individual an effective amount of an anti-Nectin-4 antibody drug conjugate of the disclosure.

Optionally, an individual treated with an anti-Nectin-4 antibody drug conjugate of the disclosure may have a cancer (e.g., a urothelial cancer, breast cancer (e.g. triple-negative breast cancer; HER2-positive cancer), non-small cell lung cancer, pancreatic cancer, ovarian cancer, gastric cancer, colorectal cancer, head and neck squamous cell carcinoma or esophageal cancer) that is resistant, has not responded, or has relapsed and/or progressed despite (e.g. during or following) surgery and/or treatment with a therapeutic agent, e.g. a chemotherapeutic agent, an antibody, an ADC or radiotherapy.

In any embodiment herein, treatment response can be defined and/or assessed according to well-known criteria, e.g. Response Evaluation Criteria In Solid Tumors (RECIST), such as version 1.1, see Eisenhauer et al. (2009) Eur. J. Cancer 45:228-247, or Immune-Related Response Criteria (irRC), see Wolchock et al. (2009) Clinical Cancer Research 15:7412-7420.

Optionally, an individual treated with an anti-Nectin-4 antibody drug conjugate of the disclosure has a tumor or cancer that displays resistance, that is not responsive to or that has progressed following treatment with a chemotherapeutic agent (e.g. a chemotherapeutic agent known to be capable of being transported by P-glycoprotein (Pgp), for example anthracyclines (doxorubicin, daunorubicin, taxanes (paclitaxel, docetaxel), Vinca alkaloids (vincristine, vinblastine, vindesine), and etoposides. Compounds recognized by Pgp are typically characterized as modestly hydrophobic (octanol-to-water partitioning coefficient, log P>1), often contain titratable protons with a net cationic charge under physiological conditions, and are predominately “natural products” with an aromatic moiety.

In some embodiments, an ADC comprising an anti-Nectin-4 antibody, antibody fragment is used or administered in the absence of combined administration of a chemotherapeutic agent.

In other embodiments, the anti-Nectin-4 antibody, antibody fragment or ADC comprising such is optionally used or administered in combination with a chemotherapeutic agent. Exemplary chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxy carbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. In one embodiment, the anti-Nectin-4 antibody, antibody fragment (or ADC comprising such) and the chemotherapeutic agent are formulated for separate administration and are administered concurrently or sequentially.

Optionally, the individual can be characterized as having cancer which has progressed, relapsed or not responded to prior treatment with a prior therapy, optionally further wherein the prior therapy comprises administration of enfortumab verdotin and/or administration of a PD-1 neutralizing agent (e.g., pembrolizumab, atezolizumab, nivolumab), optionally wherein the prior therapy is a chemotherapeutic agent.

Optionally, in any embodiment, the individual can be characterized being ineligible for treatment with enfortumab verdotin, and/or as having cancer which is not suitable or indicated for treatment with enfortumab verdotin.

Exemplary treatment protocols for treating a human with an anti-Nectin-4 antibody conjugated to an camptothecin derivative molecule include, for example, administering to the patient an effective amount of an anti-Nectin-4 antibody drug conjugate of the disclosure, wherein the method comprises at least one administration cycle in which at least one dose of the anti-Nectin-4 antibody conjugated to a camptothecin derivative molecule is administered at a dose of 0.1-10 mg/kg body weight, 0.1-5 mg/kg body weight, 0.1-1 mg/kg body weight, 1-10 mg/kg body weight or 1-5 mg/kg body weight. In one embodiment, a plurality of doses are administered, e.g. at least 2, 3, 4, 5, 6, 8, 10 doses. In one embodiment, administration of doses are separated by at least 2, 3 or 4 weeks. In one embodiment, the administration cycle is between 2 weeks and 8 weeks, or is at least 4, 6, 8 or 16 weeks.

In one embodiment the anti-Nectin-4 antibody drug conjugate of the disclosure is administered by i.v.

### EXAMPLES

### Example 1: Human Tumor Cells Co-Expressing Her2 and Nectin-4

A study of HER2 and Nectin-4 gene expression was carried out using The Cancer Genome Atlas (a collaboration between the National Cancer Institute and National Human Genome Research Institute) based on multi-dimensional maps of the key genomic changes in different types of cancer. Significant correlations of HER2 and Nectin-4 expression was observed in particular in samples from pancreatic cancer, lung adenocarcinoma patients, breast cancer and bladder cancer patients. The highest correlation observed was pancreatic cancer, with correlation values: Spearman 0.71 and Pearson 0.78.

HER2 and Nectin-4 expression on SUM185 and SUM190 human breast cancer tumor cell lines (Biovit Inc.) was determined by flow cytometry. SUM185 originates from a pleural effusion of a patient with ER negative, PR negative and HER2 positive anaplastic carcinoma of the breast. The cell line over expresses Her2. SUM190 originates from a primary tumor from a patient with ER negative, PR negative and HER2 positive (amplified) breast cancer. Tumor cells were stained with anti-Nectin-4 antibody (ASG-22ME modified as a human IgG1 isotype containing a N297Q mutation having reduced Fc gamma receptor binding) or Anti-Her2 antibody (trastuzumab modified as human IgG1 isotype containing a N297Q mutation reduced Fc gamma receptor binding), as well as isotype control, at 10 μg/ml (at 4° C.), followed by PE conjugated polyclonal goat anti human antibodies at a dilution of 1:200. Samples were analyzed by cytofluorometric analysis with Canto II (HTS).

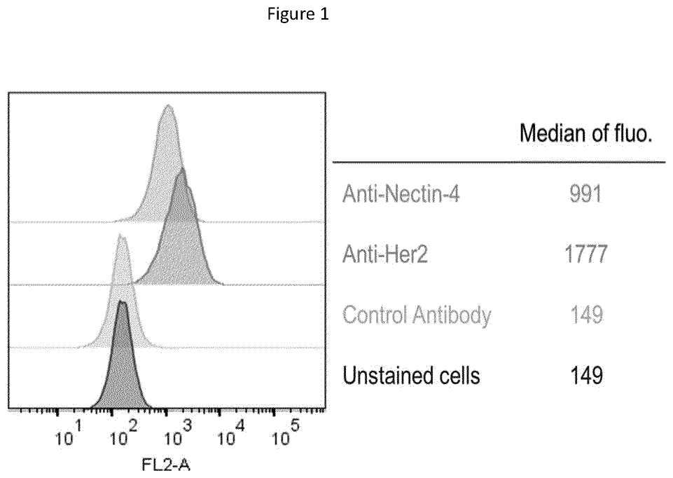

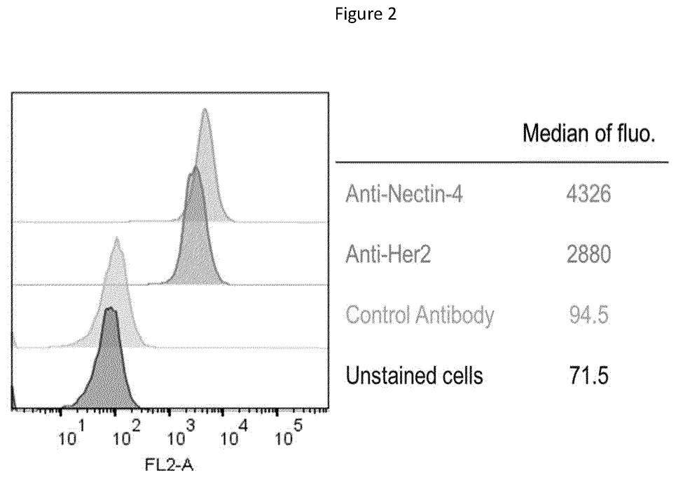

Representative results are shown in FIG. 1 for SUM 190 human breast cancer tumor cells and in FIG. 2 for SUM185 human breast cancer tumor cells. MFI: Median of fluorescence intensity. The SUM190 tumor cells expressed HER2 at low to moderate levels (median fluorescence units 1777) as well as Nectin-4 at lower levels (median 991 fluorescence units). The SUM185 cells expressed HER2 at moderate to high levels (median fluorescence units 2880) as well as Nectin-4 at higher levels (median 4326 fluorescence units).

| | HER2 (MFI) | Nectin-4 (MFI) |
|---|---|---|
| SUM185 | 2880 | 4326 |
| SUM190 | 1777 | 991 |

### Example 2: Efficacy of Anti-Nectin-4 ADCs Functionalized with Camptothecin Derivatives on HER2+ Nectin4+ Human Tumor Cells

Anti-Nectin-4 antibody-drug conjugates were prepared and compared to trastuzumab antibody-drug conjugates for efficacy on HER2+Nectin4+human tumor cells. The anti-Nectin-4 antibody-drug conjugates were prepared having the V_{H} and V_{L} of SEQ ID NOS: 28 and 29 as human IgG1 isotype.

N41 VH:
(SEQ ID NO: 28)
QVQLKQSGPGLVQPSQSLSITCTVSGFSLTNYGVHWVRQSPGKGLEWLGV
IWSGGSTDYNAAFISRLSISKDTSKSQVFFKMNSLQADDTAIYYCARELI
HAMDNWGQGTSVTVSS.
N41 VL:
(SEQ ID NO: 29)
DIQMTQSPASLSVSVGETVTITCRASENIYSNLAWYQQKQGNSPQLLVFA
ATNLADGVPSRFSGSGSGTQYSLKINSLQSEDEGTYYCQHFWGTPTFGGG
TKLEIK.

Anti-HER2 antibody-drug conjugates were prepared having the heavy and light chains of trastuzumab (human IgG1 isotype). Both the anti-Nectin-4 and the anti-HER2 antibodies were each conjugated stochastically to a linker-camptothecin derivative via cysteine residues in the antibody after partial reduction of interchain disulfide. A range of 2-10 molar equivalents of reducing agent tris (2-carboxyethyl) phosphine hydrochloride was incubated with antibody (3 mg/mL) for 2 h under agitation (350-400 rpm, +37° C.) to reduce disulfides. Conjugation of the linker-toxin was carried out by addition of a molar excess of linker-toxin at 9.2 or 12 molar equivalents incubated overnight on a stirring wheel at +37° C. The resulting ADCs had an average drug loading (drug:antibody ratio) of about 8. In a further example, anti-Nectin-4 antibodies were also conjugated to a second camptothecin (SN-38) containing linker using the same methods. The ADCs used in this Example are as follows.

N4 ADC1: Anti-Nectin-4 conjugated to a linker having the structure:

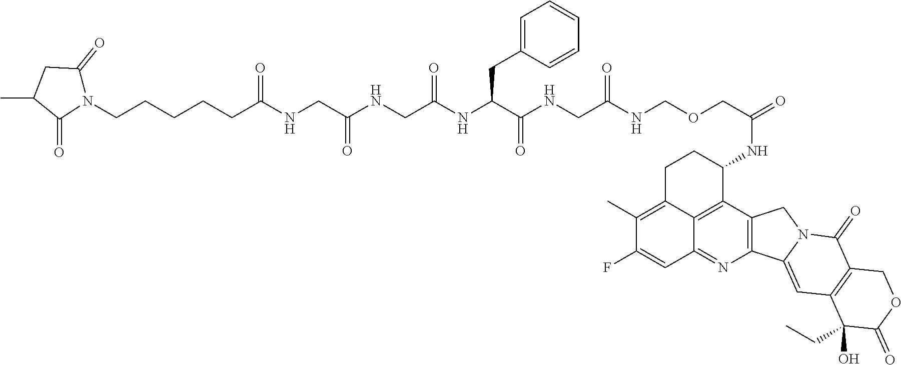 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

Her2 ADC1: Anti-HER2 conjugated to a linker having the structure:

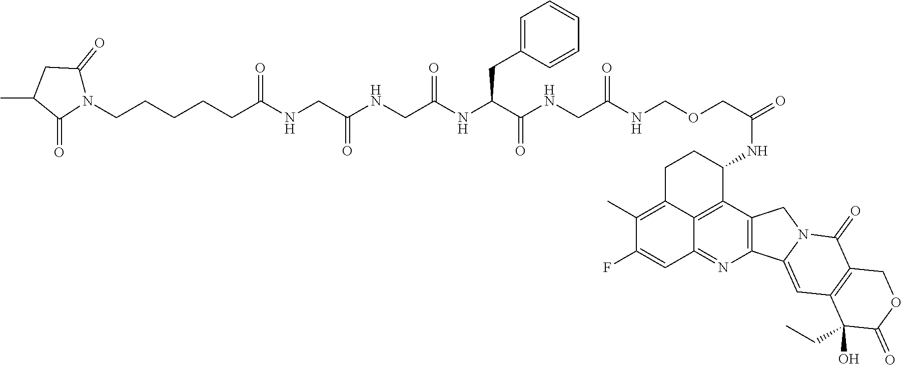 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

N4 ADC2: Anti-Nectin-4 conjugated to a linker having the structure:

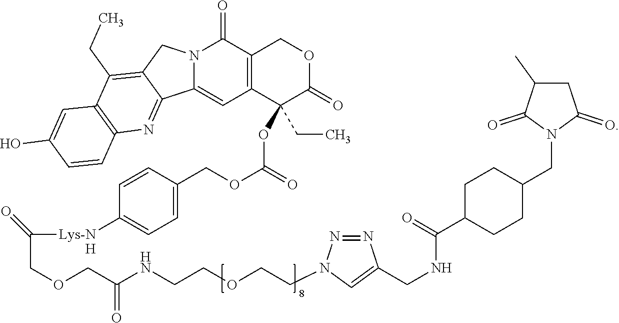

The resulting ADCs were tested for their ability to induce death of Nectin-4/HER2 expressing SUM190, MCF-7 tumor cells of Example 1. Briefly, cells were plated cells in 96 well plates (V=80 μl). N4 ADC1 and HER2 ADC1 or human IgG1-isotype control (IC)-linker-toxin or medium (conc. 5×) were tested in 1:2 serial dilution starting from (530 nM to 30 nM) and in a 1:5 serial dilution (7 nM to 7×10−2 nM) for the N4 ADC1 and isotype control. N4 ADC2 and isotype control were tested in 1:10 serial dilution starting from (530 nM to 5.3 10-2). The ADCs' ability to cause cell death was determined by assessing confluence using Incucyte S3-2 apparatus; viability at day 6 after treatment was determined using the Cell Titer Glo™ (CTG) assay with an Enspire2 apparatus. IC₅₀ values for each ADC was determined using Luminescent Cell Viability at day 6 data with GraphPad Prism8. Experiments were repeated twice.

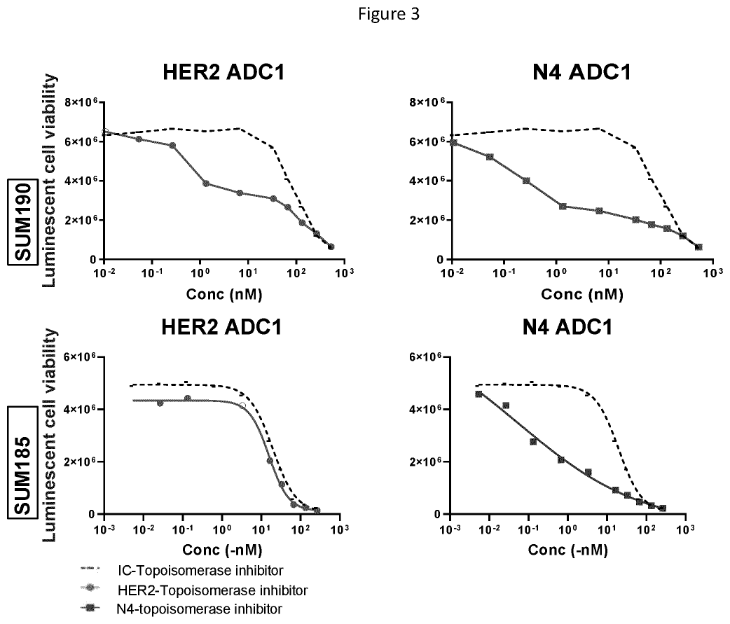

Representative results are shown in FIG. 3. The two right hand panels of FIG. 3 shows that the N4 ADC1 (anti-Nectin-4) was effective in causing the death of tumor cells, with N4 ADC1 shown as solid line with squares and the isotype control as dashed line. The two left hand panels of FIG. 3 shows the efficacy of the HER2 ADC1 (anti-HER2) in the same respective cells, with HER2 ADC1 shown as solid lines with dots and the isotype control as dashed line. IC₅₀ values are summarized in the table, below. N4 ADC1 was particularly potent even in the SUM185 cells characterized by much lower Nectin-4 surface expression (about 4-fold lower surface Nectin-4 in SUM185 than in SUM190). The N4 ADC2 (anti-Nectin-4) bearing the camptothecin derivative SN38 showed good potency as well (see IC₅₀ Table, below).

TABLE
IC₅₀ values of ADCs

| | SUM 190 | SUM 185 |
|---|---|---|
| ADC | IC₅₀ (nM) | IC₅₀ (nM) |
| Anti-Nectin-4 ADC (N4 ADC1) | 0.1 | 0.05 |
| Anti-Nectin-4 SN-38 ADC (N4 ADC2) | 0.4 | Not tested |
| Anti-HER2 ADC (Her2 ADC1) | 0.6 | 2 |

The results show that anti-Nectin-4 ADC (N4 ADC1) was considerably more potent than the anti-HER2 ADC (Her2 ADC1) in the SUM185 that express Nectin-4, with an IC₅₀ that was 40 times lower for the Nectin-4 ADCs. Of note, the SUM185 cells express Nectin-4 at relatively high levels, with Nectin-4 expression levels about twice that of HER2 in these cells (see Example 1). Interestingly, however, when the anti-Nectin-4 (N4 ADC1) and anti-HER2 (HER-2 ADC1) ADCs were tested in the SUM190 cells that have far lower levels of Nectin-4 surface expression, the anti-Nectin-4 ADC (N4 ADC1) remained highly potent. In these SUM190 cells the Nectin-4 surface expression was only half that of the HER2, yet the anti-Nectin-4 camptothecin ADCs remained at least as potent, or possibly more potent, than the anti-HER2 ADCs, with an IC₅₀ that was 6-fold lower for the anti-Nectin-4 ADCs than for the anti-anti-Her2 ADCs.

The combination of intracellularly cleavable peptide linker together with the camptothecin derivative compounds may therefore represent an effective means for eliminating Nectin-4-expressing tumor cells, including those characterized by lower Nectin-4 expression levels, without improved off-target toxicity compared to the most widely used cytotoxic agents such as pyrrolobenzodiazepines and auristatins. The anti-Nectin-4 ADCs may also provide a valuable approach to treating HER2-positive cancers, including but not limited to HER2-low and/or HER2-moderate expressing cancers.

### Example 3: Efficacy of Anti-Nectin-4 Camptothecin-Derivative ADCs in Combination with Anti-HER2 ADCs

Anti-Nectin-4 ADCs (N4 ADC1) and anti-Her2 ADCs (Her2 ADC1) were tested to evaluate their ability to cause tumor cell death when used in combination.

The anti-Nectin-4 camptothecin-derivative ADCs used were the N4 ADC1 as shown in Example 2. The anti-Her2 ADCs were also those used in Example 2 (Her2 ADC1). The ADC's ability to cause cell death was determined by assessing confluence and determining viability of SUM190 cells at day 6 as described in Example 2. Results showed that the combination of anti-Nectin-4 ADCs and anti-Her2 ADCs had an improved potency (lower IC₅₀) in causing the death of the Nectin-4+ Her2+ tumor cells compared to either ADC used alone.

### Example 4: Generation of a Panel of New Anti-Nectin-4 Antibodies

### Immunization

Balb/c mice were immunized with recombinant human Nectin-4 protein having the Nectin-4 amino acid sequence fragment below fused to a C-terminal 6-His tag:

(SEQ ID NO: 2)
GRCPAGELETSDVVTVVLGQDAKLPCFYRGDSGEQVGQVAWARVDAGEGA
QELALLHSKYGLHVSPAYEGRVEQPPPPRNPLDGSVLLRNAVQADEGEYE
CRVSTFPAGSFQARLRLRVLVPPLPSLNPGPALEEGQGLTLAASCTAEGS
PAPSVTWDTEVKGTTSSRSFKHSRSAAVTSEFHLVPSRSMNGQPLTCVVS
HPGLLQDQRITHILHVSFLAEASVRGLEDQNLWHIGREGAMLKCLSEGQP
PPSYNWTRLDGPLPSGVRVDGDTLGFPPLTTEHSGIYVCHVSNEFSSRDS
QVTVDVLDPQEDSGKQVDLVSASVV

After the second IP injection (IP2), sera of immunized mice were recovered in order to perform titrations. These titrations allowed to determine the titers of each sera and to select the best responder mice for fusion. Serum titrations were done on a CHO cell line made to express at its surface full-length human Nectin-4 (the huNectin4 CI.3C₈ cell line described below).

No background signal was observed with pre-immune sera. All immunized mice responded weakly to the immunization after two IP injections and the EC₅₀ was not calculable as the titration curves did not reach the plateau phase. According to the curves, the mice #2, #3 and #4 exhibited the higher reactivity to huNectin4 and were used for the fusion process.

The hybridomas generated by the fusion process were seeded in methylcellulose semi-solid medium-containing one well plates and incubated for 11-13 days at 37° C., 10% CO2. The growing colonies were picked and distributed into flat bottom 96 well plates. After 5-7 days the hybridoma supernatants were harvested for the flow cytometry screening #1.

Flow Cytometry Screening #1

plates of hybridoma supernatants were tested for binding on the wild-type human Nectin-4 expressing CHO cell line by flow cytometry. Briefly, Nectin-4 expressing cells were used at 0.2×106 cells by well in a buffer of PBS, BSA 0.5%, EDTA 5 mM. The first antibody was incubated with cells for 1h at +5±3° C. in 50 μL of supernatant. Control antibody included isotype Enfortumab T1-1. Cells were washed three times in staining buffer and bound anti-Nectin-4 antibodies were revealed with secondary antibody incubated at 30 min at +5±3° C., using goat anti-mouse IgG Fc specific for hybridoma supernatants and goat anti-human IgG Fc specific-PE for the control antibody. Acquisition HTFC flow cytometer (Intellicyt), with data analysis on ForeCyt™ software.

All hybridomas giving a positive signal, including at least a weak positive signal, were selected for further characterization. In order to rank the hybridomas after this 1st screening test, their profile were noted from +/− to +++according to the binding level. 35 clones were selected and transferred in a new plate for the 2nd flow cytometry screening assay.

Flow Cytometry Screening #2

In order to validate the results and the selection of the first screen, it was necessary to perform a second screen on the 35 clones selected based on the same method of screening #1. A total of 19 clones were confirmed to be positive on human Nectin-4 expressing cell line by flow cytometry. Four clones with lower MFI were also kept. These hybridomas were amplified up to 1 mL in a 24 well plate. The supernatants were harvested, and cells were frozen in RA1 lysis buffer for preservation of RNAs.

In summary, almost 1000 hybridoma supernatants binding properties were evaluated by flow cytometry on a human Nectin-4-expressing CHO cell line. After 2 rounds of screening, 23 hybridomas were selected for further characterization and K_{off} determination.

K_{off} Screening

A K_{off} screening was performed on the 23 selected clones. This test allows determination of the dissociation rate for each antibody present in the supernatant of hybridomas. The protein used for this test was the monomeric huNectin4-his-BirA protein having the amino acid sequence as follows:

(SEQ ID NO: 30)
GELETSDVVTVVLGQDAKLPCFYRGDSGEQVGQVAWARVDAGEGAQELALLHSKYGLHVS
PAYEGRVEQPPPPRNPLDGSVLLRNAVQADEGEYECRVSTFPAGSFQARLRLRVLVPPLPS
LNPGPALEEGQGLTLAASCTAEGSPAPSVTWDTEVKGTTSSRSFKHSRSAAVTSEFHLVPS
RSMNGQPLTCVVSHPGLLQDQRITHILHVSFLAEASVRGLEDQNLWHIGREGAMLKCLSEG
QPPPSYNWTRLDGPLPSGVRVDGDTLGFPPLTTEHSGIYVCHVSNEFSSRDSQVTVDVLDP
QEDSGKQVDLVSASHHHHHHLHHILDAQKMVWNHR

Briefly, AMC anti-mouse Fc Biosensors (Fortebio Corp.) were used, with equilibration with 200 μL/well by dipping in Kinetic Buffer 1×(KB1X) for 10 minutes then in RPMI medium for 10 minutes, dilution of selected antibody supernatant ⅓ in KB1X in a final volume of 200 μL, and dilution at 100 nM of recombinant protein huNectin4-BirA protein in KB1X. The steps are described in the table below, where for each step a 96 well plate was prepared (200 μL of each buffer). Results were acquired on Fortebio Data Analysis.

| Step | Time (s) | Buffer |
|---|---|---|
| 1. Baseline | 60 | KB1X |
| 2. Load | 300 | Diluted SN |
| 3. Baseline | 60 | KB1X |
| 4. Association | 180 | Diluted protein |
| 5. Dissociation | 300 | KB1X |
| 6. Regeneration | 5 (x3) | Glycine 10 Mm pH 1.8 and PBS1X. |

The screening #1, #2 resulted in the selection of nine antibodies for the cloning process. The K_{off} screening showed that all tested Abs had a high K_{off} (range from 3.75E-3 to 5.88E-3 s-1) indicating that the dissociation of the antibody/protein interaction is fast for these antibodies.

Binding to Anchored Nectin-4 Domain-Deletion Proteins

The new antibodies were produced recombinantly as human IgG1 isotypes (used in further testing hereinafter), and tested together with known comparator antibodies enfortumab and N4 were assessed for their ability to bind different domains on human Nectin-4. Enfortumab (ASG-22ME), the antibody component of FDA-approved ADC enfortumab verdotin, has been demonstrated to have high internalization ability and potent anti-tumor activity as an ADC. Antibody N41 has high internalization ability and potent anti-tumor activity as an ADC. Both enfortumab and N41 are believed to bind the membrane distal the Ig-like V type domain of Nectin-4.

The wild-type huNectin4 protein is composed of three extracellular Ig-like domains (V, C₁ and C₂) summarized in Table 1 below.

TABLE 1

| Designations | Positions | Description | Amino acid sequence |
|---|---|---|---|
| V domain | 32-144 | Ig-like V | GELETSDVVTVVLGQDAKLPCFYRGDSGEQVG |
| | | type | QVAWARVDAGEGAQELALLHSKYGLHVSPAYE |
| | | | GRVEQPPPPRNPLDGSVLLRNAVQADEGEYEC |
| | | | RVSTFPAGSFQARLRLR (SEQ ID NO: 3) |
| C1 domain | 148-237 | Ig-like C2 | PPLPSLNPGPALEEGQGLTLAASCTAEGSPAP |
| | | type 1 | SVTWDTEVKGTTSSRSFKHSRSAAVTSEFHLV |
| | | | PSRSMNGQPLTCVVSHPGLLQDQRIT |
| | | | (SEQ ID NO: 4) |
| C2 domain | 248-331 | Ig-like C2 | ASVRGLEDQNLWHIGREGAMLKCLSEGQPPPS |
| | | type 2 | YNWTRLDGPLPSGVRVDGDTLGFPPLTTEHSG |
| | | | IYVCHVSNEFSSRDSQVTVDVLDPQEDSGKQ |
| | | | VDLVSASV (SEQ ID NO: 5) |

The characterization of the binding of the antibodies on Nectin-4 domains was performed by flow cytometry using cells made to express the wild-type human Nectin-4 protein and cells made to express a modified Nectin-4 having the Ig-like C₂ type 1 and the Ig-like C₂ type 2 domains and lacking the Ig-like V domain (the C1C2 construct). The latter protein additionally bears a V5 tag for flow cytometry cell sorting, and cells were used as Nectin4-C1C2-V5 sorted cells.

Wild-type Nectin-4 (the CI.3C8 cell line), a C2 construct (containing the Ig-like C2 type 2 domain and lacking both the V and the C1 domains) and the C1C2 construct allow the determination of whether test antibodies bind to the V or different C domains. Briefly, nucleic acid sequences encoding different human Nectin-4 domains were amplified by PCR. The PCR products were inserted into an expression vector at appropriate restriction sites. A leader peptide, and for C1C2 and C2 an N-terminal V5 tag having the amino acid sequence GKPIPNPLLGLDST (SEQ ID NO: 41), were added, and expression at the surface of cells was confirmed by flow cytometry. The amino acid sequences of the resulting different human Nectin-4 domain fragment-containing proteins are shown below (V5 tag underlined). The vectors were then transfected into the CHO cell line to obtain stable clones expressing the different Nectin-4 domain proteins at the cell surface.

Nectin-4 amino acid sequence in the huNectin4 Cl.3C8
cell line (wild type Nectin-4):
(SEQ ID NO: 31)
GELETSDVVTVVLGQDAKLPCFYRGDSGEQVGQVAWARVDAGEGAQELALLHSKYGLHVS
PAYEGRVEQPPPPRNPLDGSVLLRNAVQADEGEYECRVSTFPAGSFQARLRLRVLVPPLPS
LNPGPALEEGQGLTLAASCTAEGSPAPSVTWDTEVKGTTSSRSFKHSRSAAVTSEFHLVPS
RSMNGQPLTCVVSHPGLLQDQRITHILHVSFLAEASVRGLEDQNLWHIGREGAMLKCLSEG
QPPPSYNWTRLDGPLPSGVRVDGDTLGFPPLTTEHSGIYVCHVSNEFSSRDSQVTVDVLDP
QEDSGKQVDLVSASVVVVGVIAALLFCLLVVVVVLMSRYHRRKAQQMTQKYEEELTLTREN
SIRRLHSHHTDPRSQPEESVGLRAEGHPDSLKDNSSCSVMSEEPEGRSYSTLTTVREIETQ
TELLSPGSGRAEEEEDQDEGIKQAMNHFVQENGTLRAKPTGNGIYINGRGHLV.
Nectin-4 amino acid sequence in the huNectin4-C1C2-V5
cell line (lacking V domain):
(SEQ ID NO: 32)
PPLPSLNPGPALEEGQGLTLAASCTAEGSPAPSVTWDTEVKGTTSSRSFKHSRSAAVTSEF
HLVPSRSMNGQPLTCVVSHPGLLQDQRITHILHVSFLAEASVRGLEDQNLWHIGREGAMLK
CLSEGQPPPSYNWTRLDGPLPSGVRVDGDTLGFPPLTTEHSGIYVCHVSNEFSSRDSQVT
VDVLDPQEDSGKQVDLVSASVVVVGVIAALLFCLLVVVVVLMSRYHRRKAQQMTQKYEEEL
TLTRENSIRRLHSHHTDPRSQPEESVGLRAEGHPDSLKDNSSCSVMSEEPEGRSYSTLTTV
REIETQTELLSPGSGRAEEEEDQDEGIKQAMNHFVQENGTLRAKPTGNGIYINGRGHLV.
Nectin-4 amino acid sequence in the huNectin4-C2-V5 cell
line (lacking V and C1 domains):
(SEQ ID NO: 33)
ASVRGLEDQNLWHIGREGAMLKCLSEGQPPPSYNWTRLDGPLPSGVRVDGDTLGFPPLTT
EHSGIYVCHVSNEFSSRDSQVTVDVLDPQEDSGKQVDLVSASVVVVGVIAALLFCLLVVVVV
LMSRYHRRKAQQMTQKYEEELTLTRENSIRRLHSHHTDPRSQPEESVGLRAEGHPDSLKD
NSSCSVMSEEPEGRSYSTLTTVREIETQTELLSPGSGRAEEEEDQDEGIKQAMNHFVQENG
TLRAKPTGNGIYINGRGHLV.

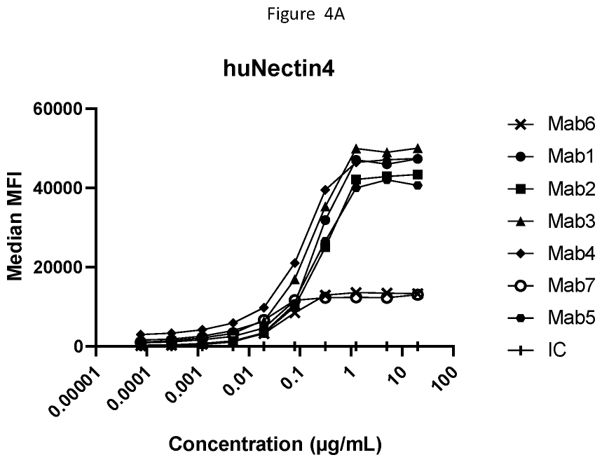

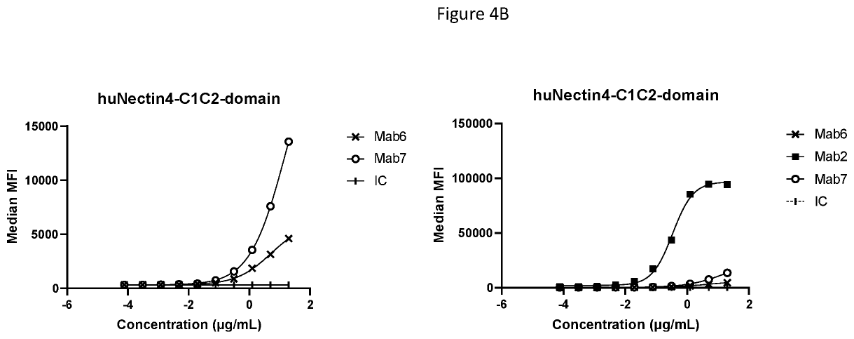

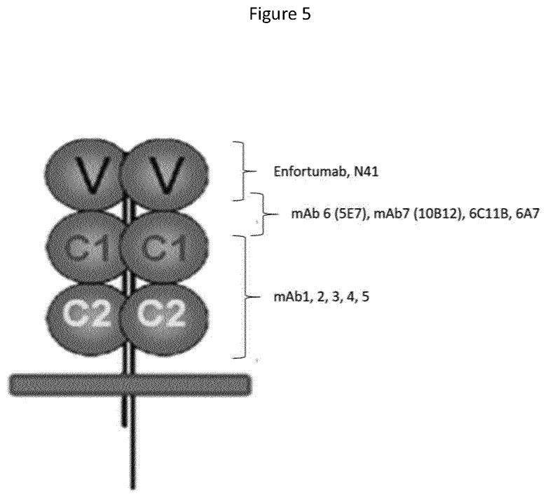

Three of the nine selected antibodies did not bind to the whole Nectin-4 protein. This result was in accordance with the K_{off} experiment showing that these three antibodies were not sufficiently concentrated in the sample to generate reliable results (Capture signal <0.3 nm). Results are shown in FIG. 4A for binding to whole (wild-type) Nectin-4 protein and in FIG. 4B for binding to C1C2 construct. For the six remaining selected antibodies, four antibodies retained full binding to the whole Nectin-4 protein as well as to the C1C2 construct lacking the Ig-like V type domain, showing that they bind within the C1 and/or C2 domains. In comparison, the gold-standard internalizing antibodies N41 and enfortumab bound to the whole Nectin-4 protein and lost all binding to the C1C2 construct. N41 and enfortumab thus bind to Nectin-4 within the V domain. Two antibodies, 10B12 (also referred to as mAb7) and 5E7 (also referred to as mAb6), showed a profile which corresponded neither to an anti-Ig-like V domain antibody nor to an anti-Ig-like C2 type 1 and/or type 2 domain antibody. At a concentration of 1 μg/mL, mAbs 1-5 displayed fluorescence (MFI) values in the range of 100000, whereas the MFI for mAbs 6 and 7 were between 5000 and 15000. Antibodies 5E7 and 10B12 thus showed a partial loss of binding to the C1C2 construct, suggesting that they bind Nectin-4 partially on the V domain and partially on the Ig-like C2 type 1 domain, with the loss of the binding to the C1C2 construct confirmed to be a decrease but not full loss of binding when repeated with cloned and purified antibodies. In view of structural analysis of the Nectin-4 protein together with antibody binding profiles to human, cynomolgus, rat and mouse Nectin-4, the antibodies 10B12 (mAb7) and 5E7 (mAb6) are believed to bind Nectin-4 at the junction of domain C1 and V. The domains of Nectin-4 and the antibodies are represented in FIG. 5.

EC₅₀ values for binding to Hu-Nectin4 IgC1C2-V5 sorted cells

| | Hu-Nectin4 IgC1C2 | | | | | | | |
|---|---|---|---|---|---|---|---|---|
| | Mab1 | Mab2 | Mab3 | Mab4 | Mab5 | Mab6 | Mab7 | IC |
| IC₅₀ (μg/ml) | 0.1895 | 0.2357 | 0.1564 | 0.1042 | 0.1981 | 0.05236 | 0.01969 | NC |

In conclusion, more than 35 antibodies were obtained and characterized, and the antibodies obtained in this immunization were essentially all antibodies that bound the Ig-like C2 type 1 and/or type 2 domains. No antibodies were obtained that were specific to the Ig-like V set domain which is associated with highly-internalizing gold-standard antibodies used in ADCs.

### Example 5: Characterization of Binding to Nectin-4 by Flow Cytometry Titration Assay

Despite the fact that no enfortumab- or N41-like anti-Ig like V set domain antibodies were obtained in Example 4, the antibodies that bound the Ig-like C2 type 1 and/or type 2 domains were not discarded and nevertheless further characterized in a flow cytometry titration assay, in an assay for their internalization capacity, and in a direct cytotoxicity assay conjugated to a topoisomerase inhibitor (the campthothecin analogue deruxtecan).

In this experiment, the ability of anti-Nectin-4 Abs to bind to Nectin-4 on SUM185 cells (expressing relatively high levels of Nectin-4) was assessed by flow cytometry. A concentration range of the antibody was incubated on top of the cells and bound anti-nectin-4 antibodies were revealed by addition of the secondary antibody “Goat anti-human H+L PE” (fluorescence was proportional to the binding of the anti-nectin-4 Abs to the cells). Briefly, SUM185 cells were detached from the flask with trypsin (37° C. for 5 min) and then washed once with media. 50 000 cells were incubated with a concentration range of test antibodies starting from 20 μg/ml (8 points, dilution factor 4) for 30 minutes at 4° C. Cells were washed three times with ice-cold SB and bound anti-nectin-4 Abs were revealed by addition of the secondary antibody “Goat anti-human H+L PE” (Jackson Immunoresearch) at 1/200 for 30 minutes on ice. Cells were washed twice with ice-cold SB. Acquisition was done on a flow cytometer.

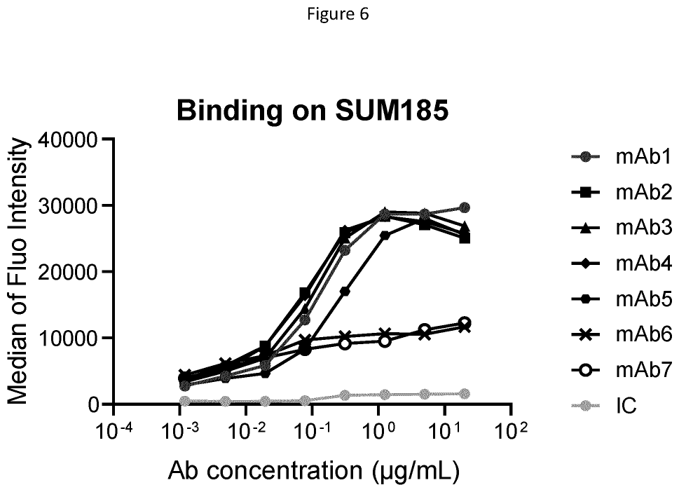

Results are shown in FIG. 6. It was observed that there were two different groups of anti-Nectin-4 Abs according to their binding profile. Generally, with the exception of two antibodies, the new antibodies of Example 4 had a similar profile to Enfortumab, showing a similarly high plateau for binding to the SUM185 cells. The exceptions were two antibodies of Example 4, 5E7 (mAb6) and 10B12 (mAb7) which had a different profile characterized by a lower plateau for binding to the SUM185 cells. FIG. 6 shows the binding profile of the different antibodies of Example 4 and isotype control (IC) on SUM185 cells. From this experiment, the C₁ and/or C₂ specific antibodies (mAbs1-5) generally appear to have a binding profile comparable to enfortumab.

### Example 6: Intracellular Internalization

The ability of the anti-Nectin-4 antibodies to induce nectin-4 internalization was assessed on SUM190 cell lines expressing lower levels of nectin-4 and in SUM185 cells expressing higher levels of Nectin-4. The internalization was indirectly determined by using the Fab-ZAP human Internalization Kit (Advanced Targeting Systems) to allow the anti-Nectin-4 antibodies to target and eliminate Nectin-4 expressing cells, with measurement of cell viability using the CTG substrate (CellTiter-Glo® Luminescent Cell Viability Assay (Promega). Fab-ZAP is a chemical conjugate of goat anti-human monovalent antibody and the ribosome-inactivating protein, saporin. The antibodies used are affinity-purified polyclonal antibodies against both the heavy and light chain of human IgG. This secondary conjugate is used to evaluate the potential of a primary antibody to internalize.

Briefly, a concentration range of the anti-Nectin-4 antibodies or the controls were incubated with SUM190 or SUM185 cells. After 45-minute incubation at 4° C., the non-bound antibodies were washed away and Fab-ZAP was added on top of the cells. The newly formed complexes (anti-nectin-4 antibody+Fab-ZAP) internalized into the cells where the Saporin was released and stopped the protein synthesis leading to cell death. The internalization capability of the antibody was indirectly determined by the cell's viability: the more efficient was the cell killing; the better was the internalization capability of the antibody.

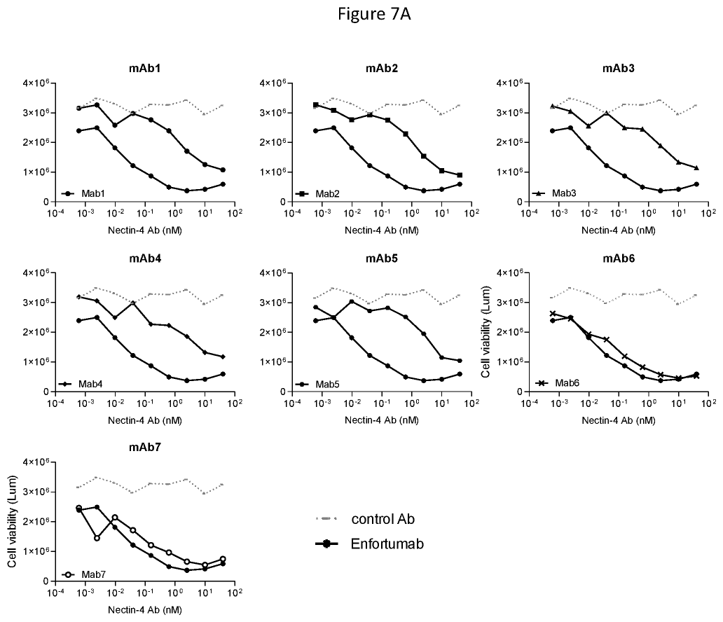

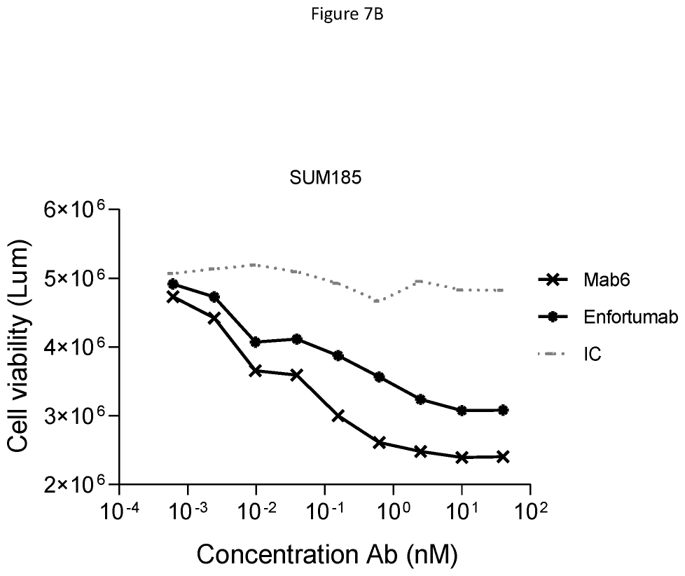

FIG. 7A shows internalization on SUM190 cells, showing luminescence vs. anti-Nectin-4 antibody concentration was plotted on graphs for each antibody, as well as enfortumab. Various of the new anti-Nectin-4 antibodies of Example 4 induced internalization of Nectin-4 and, consecutively, the death of the cells compared to the negative control (IC). However, the five antibodies that bound the Ig-like C₂ type 1 and/or type 2 domains without binding to the Ig-like V set domain were all considerably less potent that enfortumab in induction of internalization, suggesting that highly-internalizing antibodies are those that bind the Ig-V set domain. However, the two non-IgV, non-C1C2 antibodies (5E7 and 10B12) were as efficient as the enfortumab in inducing internalization.

FIG. 7B shows internalization for mAb6 compared to enfortumab on SUM185 cells, showing luminescence vs. anti-Nectin-4 antibody concentration was plotted on graphs for each antibody. Antibody 5E7 (mAb6) was more efficient than enfortumab in inducing internalization in the SUM185 cells.

### Example 7: In Vitro Cytotoxicity of New Antibodies as ADCs on Tumor Cell Lines Nectin-4 Low/SUM190 Breast Cancer Model

We assessed the ability of the 5E7 antibody (mAb6) conjugated with camptothecin analogues (Dxd or Exatecan) to kill SUM190 cells. In this experiment, the 5E7 antibody was tested along with anti-Ig-like V domain antibodies Enfortumab and N41 and a control antibody conjugated with the same toxin at equivalent drug to antibody ratios. A first ADC was prepared in which antibodies were conjugated at 8 toxins per antibody (DAR=8) to a camptothecin analogue (Dxd) via a linker comprising an intracellularly cleavable tetrapeptide linker (GGFG) having the structure shown below, referred to as ggfg-Dxd:

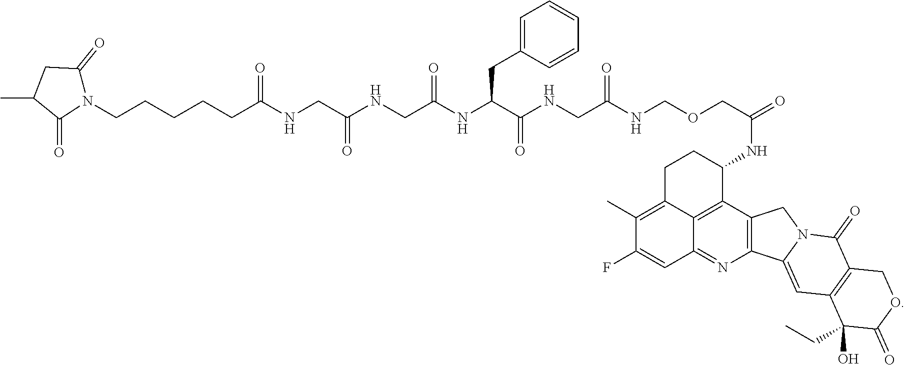 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

A second ADC was prepared in which antibodies were conjugated at 8 toxins per antibody (DAR=8) to another camptothecin analogue (exatecan) via the cleavable linker having the structure below, referred to as PEG (8U)-Val-Ala-PAB-Exatecan:

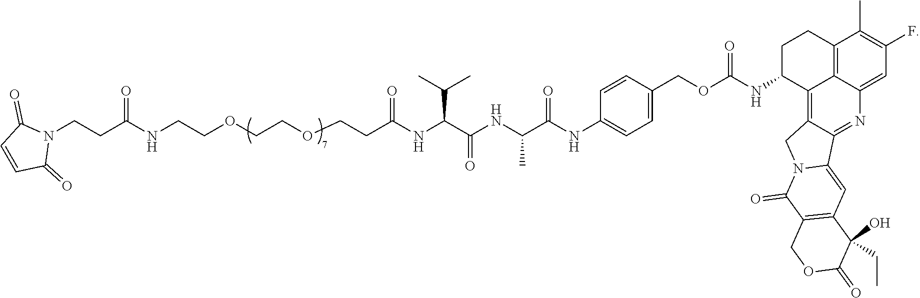

Briefly, dose-ranges of each tested Ab (starting point 150 nM, dilution factor 2 for 3 points, then 5 for 5 points) were incubated with cells for 5 days before cell viability measurement by addition of CTG substrate. Luminescence vs. Ab concentration was plotted on graph.

For each ADC, a concentration range of ADC was incubated with nectin-4-expressing cells. After incubation, CTG substrate was added at 1/1 ratio and the luminescent signal was read with a plate reader (Enspire). It allowed quantifying the ATP present (indicator of metabolically active cells) which was proportional to cell viability.

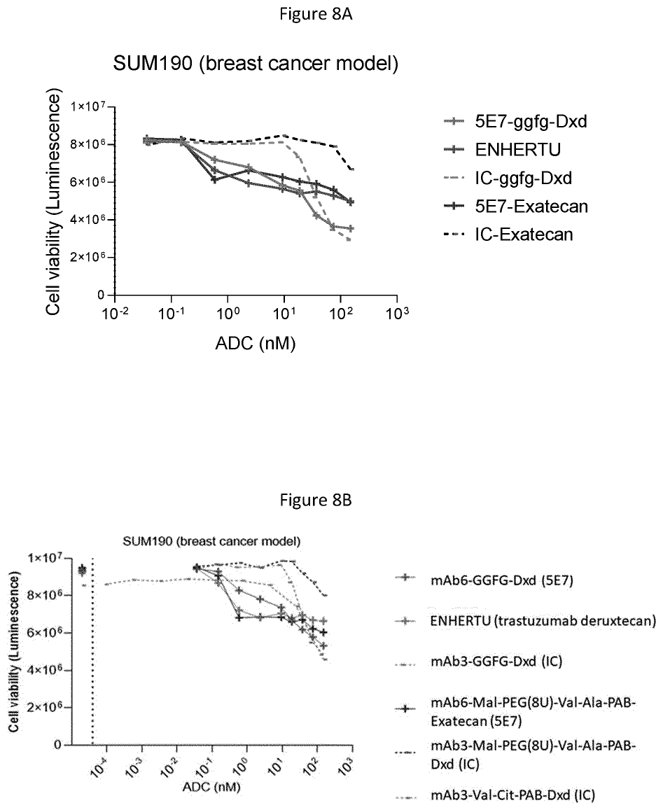

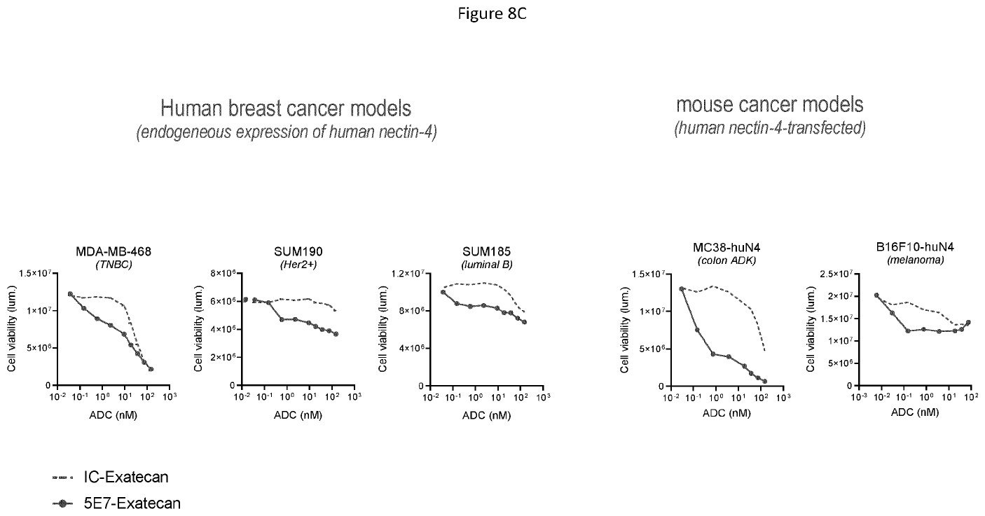

Results are shown in FIGS. 8A and 8B. FIG. 8A shows killing of human breast cancer cells by the mAb6 (5E7) antibody conjugated to the camptothecin analogues Dxd (via a GGFG-Dxd linker) or exatecan (via the (PEG (8U)-Val-Ala-PAB-Exatecan linker), along with isotype control antibodies (IC), all at equivalent drug to antibody ratios (DAR=8), and compared to compared to V-domain binding Enhertu™ (trastuzumab deruxtecan (anti-HER2)). FIG. 8B shows killing of human breast cancer cells by the mab6 (5E7) conjugated with the same camptothecin analogue-comprising linkers, along with immunocontrol conjugated with the camptothecin analogues, mAb3 conjugated to camptothecin analogue-comprising linkers at the same DAR, and Enhertu™

The three Nectin-4-targeted ADCs reduced the cell viability more efficiently than the non-targeted ADC (IC-GGFG-camptothecin). Enhertu™ and 5E7 conjugated either camptothecin analogue-comprising linker were more potent that mAb3 conjugated to camptothecin analogue no matter what linker used.

SUM185, MDA-MB-468, MC38 and B16F10 cell lines

Antibody 5E7 conjugated to exatecan (via the (PEG (8U)-Val-Ala-PAB-Exatecan linker) was further tested for cell killing on other cancer cells lines. FIG. 8C shows efficacy of “5E7-exatecan” (5E7 conjugated to the exatecan linker (PEG (8U)-Val-Ala-PAB-Exatecan) is capable of causing the death of HER-2 and Nectin-4 expressing SUM185 and SUM190, as well as MDA-MB-468 (TNBC) human tumor cells and human Nectin-4-expressing MC38 (colon cancer), and B16F10 (melanoma) murine tumor cells. EC₅₀ values for cell viability are shown below.

| | Cell line | | | | |
|---|---|---|---|---|---|
| | MDA-MB- | | | MC38- | B16F10- |
| | 468 | SUM185 | SUM190 | huN4 | huN4 |
| EC₅₀ (nM) | 1.5E+05 | 3E+05 | 7E+05 | 2.3E+5 | 9.8E+5 |

### Example 8: Modified Flow Cytometry Titration Assay to Characterize Binding by Anti-IgVC1 Antibodies

Due to the specific domain of Nectin-4 receptor that was recognized by the antibody 5E7, the flow cytometry titration protocol of Example 5 was modified. It was hypothesized that in Example 5, trypsin is cleaving Nectin-4 on the tumor cells in way that affects or destroys the epitope of the anti-VC1 domain antibody 5E7. In order to explore this possibility, following detachment with trypsin, the tumor cells were grown overnight in order to allow re-expression of Nectin-4.

SUM190 nectin-4 expressing cells were detached from the flask with trypsin (37° C. for 5 min) and then washed once with media. 50,000 cells per well were seeded into a 96 well flat-bottom plate and incubated overnight in the incubator at 37° C. Dose range of the antibodies were prepared starting at 20 μg/ml (3 points, dilution factor 20) and incubated on the washed-cells for 60 minutes at 4° C. Cells were washed twice on ice with ice-cold SB (staining buffer) and bound anti-nectin-4 antibodies were revealed by addition of the secondary antibody “Goat anti-human H+L PE” (Jackson Immunoresearch) at 1/200 for 30 minutes on ice. Cells were detached by pipetting up and down several times and transferred to a 96-well U-bottom plate and washed three times with SB. Acquisition was done on a flow cytometer.

### Example 9: In Vivo Efficacy of ADCs in a Mouse Model of Human Breast Cancer (Nectin-4 Low/SUM 190 Model)

We compared the efficacy of the 5E7 antibody as a camptothecin ADC, in comparison to enfortumab and N41, in a mouse model of human breast cancer. In this experiment, the 5E7 antibody was tested together with enfortumab and N41 and a control Ab conjugated to the ggfg-Dxd linker at equivalent drug to antibody ratios of 8 toxins per antibody (DAR=8).

SUM190 cells were subcutaneously engrafted in CB17—SCID immunodeficient mice at a dose of 0.5 million cells in 100 μl of Matrigel with growth factor diluted at ½ in PBS. When tumors reached a volume between 195 and 250 mm3, mice were randomized into groups of 9 mice for intravenous treatment with a single injection of 3 mg/kg of camptothecin ADCs. Tumor growth were followed twice a week. Kaplan Meier survival curves were established by using GraphPad Prism V7 software according to the following criteria: When the tumor volume reached 1500 mm3, mice were euthanized and considered dead on the day of sacrifice (D).

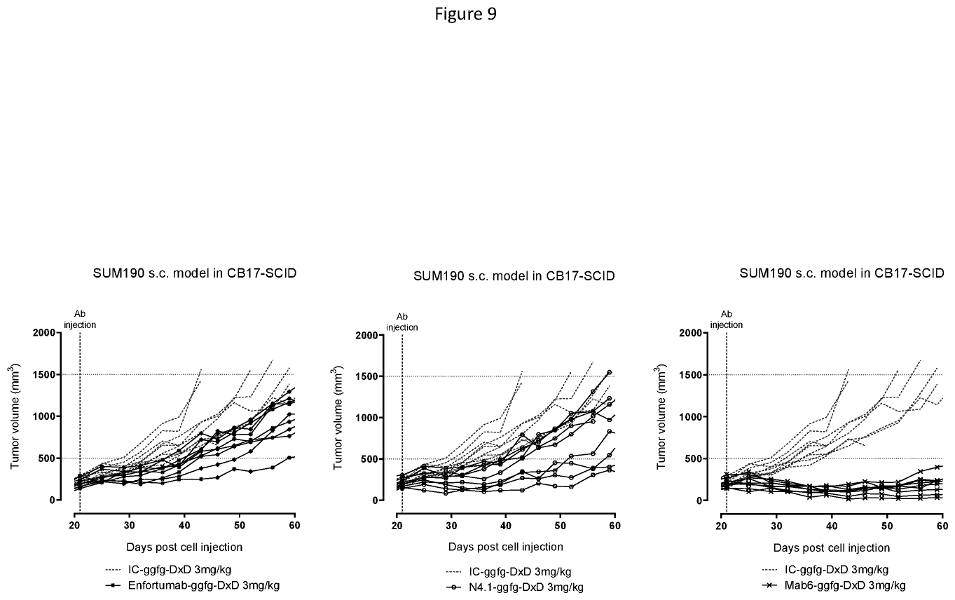

When tumors showed signs of necrosis, mice were euthanized and considered dead on the same day (D) (indicated with red star on the graphs of individual tumor growth). Results showed that at the 10 mg/kg dose all ADC were similarly efficient in preventing increase in tumor volume. However, at the lower dose of ADC (3 mg/kg), antibody 5E7 showed a strong ability to prevent tumor growth while both N41 and enfortumab no longer showed the ability to control tumor growth. Results for the 3 mg/kg dose are shown in FIG. 9.

### Example 10: Comparative In Vitro Efficacy of ADCs in a Breast Cancer Model of Drug Resistance (Human Breast Cancer, HER2/Nectin-4 High/SUM185 Model)

We next assessed the ability of the 5E7 Ab conjugated with a camptothecin analogue to kill SUM185 cells, compared to PADCEV™ (enfortumab verdotin) and ENHERTU™. This setting is used as a model of anti-HER2 resistance. SUM185 cells express Nectin-4 at relatively high levels, with Nectin-4 expression levels about twice that of HER2 in these cells (see Example 1). In this experiment, the 5E7 antibody was tested as an ADC having DAR=8 along with PADCEV™ (DAR=4, specification for FDA-approved PADCEV™) and control antibodies all conjugated with the same toxin at equivalent drug to antibody ratios. 5E7 was conjugated with a camptothecin analogue Dxd via the ggfg-Dxd linker.

Briefly, dose-ranges of each tested Ab (starting point 150 nM, dilution factor 2 for 3 points, then 5 for 5 points) were incubated on cells for 5 days before cell viability measurement by addition of CTG substrate. Luminescence vs. Ab concentration was plotted on graph.

For each ADC, a concentration range of ADC was incubated with nectin-4-expressing cells. After incubation, CTG substrate was added at 1:1 ratio and the luminescent signal was read with a plate reader (Enspire), allowing quantification of the ATP present (indicator of metabolically active cells) which was proportional to cell viability.

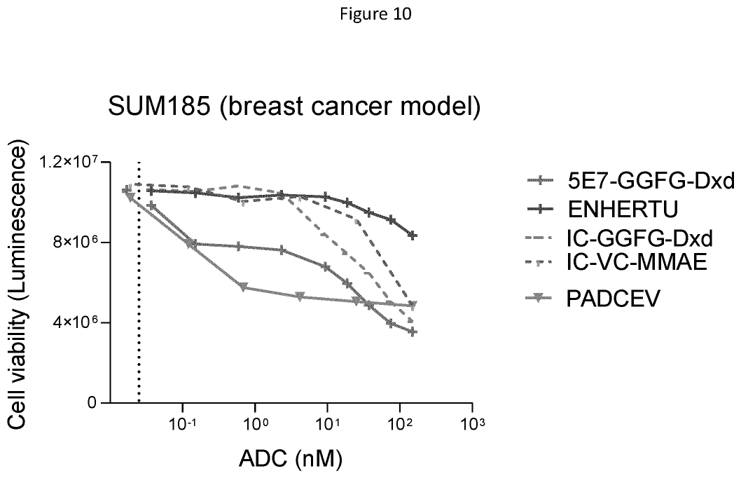

Results are shown in FIG. 10. 5E7-ggfg-Dxd and PADCEV™ were able to kill the SUM185 more efficiently than ENHERTU™. In each case, the Nectin 4-targeted ADCs reduced the cell viability more efficiently than their non-targeted ADC (IC) counterpart.

### Example 11: Characterization of Species Cross-Reactivity of Anti-Nectin-4 Antibodies

Anti-Nectin-4 antibodies were tested by flow cytometry for binding to different CHO cell lines made to express respectively the mouse, cynomolgus and rat Nectin-4 protein (including an N-terminal V5 tag not shown in the sequences below).

The mature amino acid sequence of the proteins expressed by the cells were as follows:

Mouse Nectin-4:
(SEQ ID NO: 34)
ELETSDVVTVVLGQDAKLPCFYRGDPDEQVGQVAWARVDPNEGIRELALLHSKYGLHVNPAYEDRVEQ
PPPPRDPLDGSVLLRNAVQADEGEYECRVSTFPAGSFQARMRLRVLVPPLPSLNPGPPLEEGQGLTLA
ASCTAEGSPAPSVTWDTEVKGTQSSRSFTHPRSAAVTSEFHLVPSRSMNGQPLTCVVSHPGLLQDRRI
THTLQVAFLAEASVRGLEDQNLWQVGREGATLKCLSEGQPPPKYNWTRLDGPLPSGVRVKGDTLGFPP
LTTEHSGVYVCHVSNELSSRDSQVTVEVLDPEDPGKQVDLVSASVIIVGVIAALLFCLLVVVVVLMSR
YHRRKAQQMTQKYEEELTLTRENSIRRLHSHHSDPRSQPEESVGLRAEGHPDSLKDNSSCSVMSEEPE
GRSYSTLTTVREIETQTELLSPGSGRTEEDDDQDEGIKQAMNHFVQENGTLRAKPTGNGIYINGRGHL
V.
Rat Nectin-4:
(SEQ ID NO: 35)
MPLSLGAEMWGPEAWLLLLFLASFTGRYSAGELETSDLVTVVLGQDAKLPCFYRGDPDEQVGQVAWAR
VDPNEGTRELALLHSKYGLHVSPAYEDRVEQPPPPRDPLDGSILLRNAVQADEGEYECRVSTFPAGSE
QARMRLRVLVPPLPSLNPGPPLEEGQGLTLAASCTAEGSPAPSVTWDTEVKGTQSSRSFKHSRSAAVT
SEFHLVPSRSMNGQPLTCVVSHPGLLQDORITHTLQVAFLAEASVRGLEDQNLWHVGREGATLKCLSE
GQPPPKYNWIRLDGPLPSGVRVKGDTLGFPPLTTEHSGVYVCHVSNELSSRASQVTVEVLDPEDPGKQ
VDLVSASVVVVGVIAALLFCLLVVVVVLMSRYHRRKAQQMTQKYEEELTLTRENSIRRLHSHHTDPRS
QPEESVGLRAEGHPDSLKDNSSCSVMSEEPEGRSYSTLTTVREIETQTELLSPGSGRTEEEDDQDEGI
KQAMNHFVQENGTLRAKPTGNGIYINGRGHLV.
Cynomolgus Nectin-4:
(SEQ ID NO: 36)
GELETSDVVTVVLGQDAKLPCFYRGDSGEQVGQVAWARADAGEGAQELALLHSKYGLHVSPAYEGRVE
QPPPPRNPLDGSVLLRNAVQADEGEYECRVSTFPAGSFQARLRLRVLVPPLPSLNPGPALEEGQGLTL
AASCTAEGSPAPSVTWDTEVKGTTSSRSFKHSRSAAVTSEFHLVPSRSMNGQPLTCVVSHPGLLQDQR
ITHILHVSFLAEASVRGLEDQNLWHVGREGAMLKCLSEGQPPPSYNWTRLDGPLPSGVRVDGDTLGFP
PLTTEHSGIYVCHVSNEFSSRDSQVTVDVLDPQEDSGKQVDLVSASVVVVGVIAALLFCLLVVVVVLM
SRYHRRKAQQMTQKYEEELTLTRENSIRRLHSHHTDPRSQPEESVGLRAEGHPDSLKDNSSCSVMSEE
PEGRSYSTLTTVREIETQTELLSPGSGRTEEEEDQDEGIKQAMNHFVQENGTLRAKPTGNGIYINGRG
HLV.

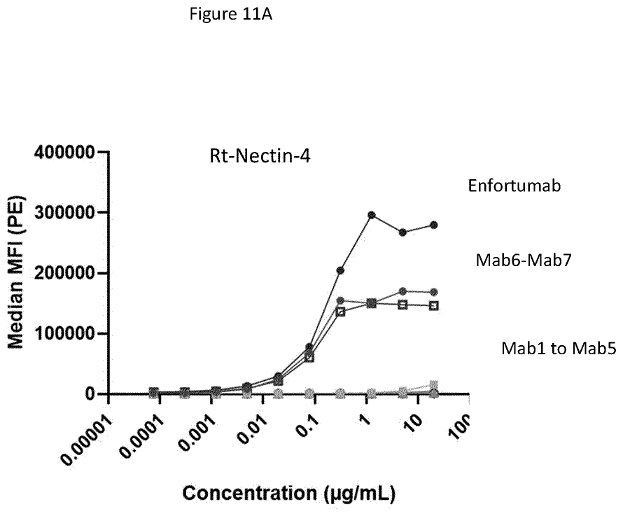

All the anti-Nectin-4 antibodies of Example 4 bound human and cynomolgus Nectin 4 proteins but lack binding to the mouse Nectin 4 protein. Solely 5E7, 10B12 showed binding to rat Nectin 4 expressing cells. FIGS. 11A and 11B show binding of anti-Nectin-4 antibodies on rat and cynomologus Nectin-4-expressing CHO cell lines.

### Example 12: Characterization of Nectin Family Cross Reactivity

Anti-Nectin-4 antibodies were tested by flow cytometry for binding to different CHO cell lines made to express respectively the human Nectin 1, Nectin 2, Nectin 3 and PVR proteins. The expression of each cell lines was controlled and validated with known respectively the anti-human Nectin1, anti-human Nectin2, anti-human Nectin3 and anti-human PVR antibodies. The mature amino acid sequence of the proteins expressed by the cells were as follows:

Nectin-1:
(SEQ ID NO: 37)
MGLAGAAGRWWGLALGLTAFFLPGVHSQVVQVNDSMYGFIGTDVVLHCSFANPLPSVKITQVTWQKST
NGSKQNVAIYNPSMGVSVLAPYRERVEFLRPSFTDGTIRLSRLELEDEGVYICEFATFPTGNRESQLN
LTVMAKPTNWIEGTQAVLRAKKGQDDKVLVATCTSANGKPPSVVSWETRLKGEAEYQEIRNPNGTVTV
ISRYRLVPSREAHQQSLACIVNYHMDRFKESLTLNVQYEPEVTIEGEDGNWYLQRMDVKLICKADANP
PATEYHWTTLNGSLPKGVEAQNRTLFFKGPINYSLAGTYICEATNPIGTRSGQVEVNITEFPYTPSPP
EHGRRAGPVPTAIIGGVAGSILLVLIVVGGIVVALRRRRHTFKGDYSTKKHVYGNGYSKAGIPQHHPP
MAQNLQYPDDSDDEKKAGPLGGSSYEEEEEEEEGGGGGERKVGGPHPKYDEDAKRPYFTVDEAEARQD
GYGDRTLGYQYDPEQLDLAENMVSQNDGSFISKKEWYV.
Nectin-2:
(SEQ ID NO: 38)
MARAAALLPSRSPPTPLLWPLLLLLLLETGAQDVRVQVLPEVRGQLGGTVELPCHLLPPVPGLYISLV
TWQRPDAPANHQNVAAFHPKMGPSFPSPKPGSERLSFVSAKQSTGQDTEAELQDATLALHGLTVEDEG
NYTCEFATFPKGSVRGMTWLRVIAKPKNQAEAQKVTFSQDPTTVALCISKEGRPPARISWLSSLDWEA
KETQVSGTLAGTVTVTSRFTLVPSGRADGVTVTCKVEHESFEEPALIPVTLSVRYPPEVSISGYDDNW
YLGRTDATLSCDVRSNPEPTGYDWSTTSGTFPTSAVAQGSQLVIHAVDSLENTTFVCTVTNAVGMGRA
EQVIFVRETPNTAGAGATGGIIGGIIAAIIATAVAATGILICRQQRKEQTLQGAEEDEDLEGPPSYKP
PTPKAKLEAQEMPSQLFTLGASEHSPLKTPYFDAGASCTEQEMPRYHELPTLEERSGPLHPGATSLGS
PIPVPPGPPAVEDVSLDLEDEEGEEEEEYLDKINPIYDALSYSSPSDSYQGKGFVMSRAMYV.
Nectin-3:
(SEQ ID NO: 39)
MARTLRPSPLCPGGGKAQLSSASLIGAGLLLQPPTPPPLLLLLFPLLLESRLCGALAGPIIVEPHVTA
VWGKNVSLKCLIEVNETITQISWEKIHGKSSQTVAVHHPQYGFSVQGEYQGRVLFKNYSLNDATITLH
NIGFSDSGKYICKAVTFPLGNAQSSTTVTVLVEPTVSLIKGPDSLIDGGNETVAAICIAATGKPVAHI
DWEGDLGEMESTTTSFPNETATIISQYKLFPTRFARGRRITCVVKHPALEKDIRYSFILDIQYAPEVS
VTGYDGNWFVGRKGVNLKCNADANPPPFKSVWSRLDGQWPDGLLASDNTLHFVHPLTENYSGVYICKV
TNSLGQRSDQKVIYISDPPTTTTLQPTIQWHPSTADIEDLATEPKKLPFPLSTLATIKDDTIATIIAS
VVGGALFIVLVSVLAGIFCYRRRRTERGDYFAKNYIPPSDMQKESQIDVLQQDELDSYPDSVKKENKN
PVNNLIRKDYLEEPEKTQWNNVENLNRFERPMDYYEDLKMGMKFVSDEHYDENEDDLVSHVDGSVISR
REWYV.
PVR (Nectin-5):
(SEQ ID NO: 40)
DVVVQAPTQVPGFLGDSVTLPCYLQVPNMEVTHVSQLTWARHGESGSMAVFHQTQGPSYSESKRLEFV
AARLGAELRNASLRMEGLRVEDEGNYTCLFVTFPQGSRSVDIWLRVLAKPONTAEVOKVOLTGEPVPM
ARCVSTGGRPPAQITWHSDLGGMPNTSQVPGELSGTVTVTSLWILVPSSQVDGKNVTCKVEHESFEKP
QLLTVNLTVYYPPEVSISGYDNNWYLGQNEATLTCDARSNPEPTGYNWSTTMGPLPPFAVAQGAQLLI
RPVDKPINTTLICNVTNALGARQAELTVQVKEGPPSEHSGISRNAIIFLVLGILVFLILLGIGIYFYW
SKCSREVLWHCHLCPSSTEHASASANGHVSYSAVSRENSSSQDPQTEGTR.

Results showed that there were no cross reactivity of the anti-Nectin-4 antibodies on members of the human Nectin family. The figure shows flow cytometry results of anti-Nectin-4 antibodies on human Nectin 1, human Nectin 2, human Nectin 3 and human PVR expressing CHO cells. No antibodies presented bind to each cell lines.

### Example 13: Affinity Determination by SPR

Antibodies were tested for binding affinity to the wild-type human Nectin-4 protein by SPR (Surface Plasmon Resonance) methods. Measurements were performed on a Biacore T200 apparatus (Biacore GE Healthcare) at 25° C. Sensorgrams were analyzed with Biacore T100 Evaluation software. A CMR Sensor Chip with immobilized Protein A at about 600 RU was used. Anti-Nectin-4 antibodies were captured onto the Protein-A chip (antibodies diluted to 1 μg/ml in HBS-EP+1× buffer and injected over 120s at 40 μL/min). Recombinant human monomeric huNectin4-his-BirA protein was injected at different concentrations in a range from 100 nM to 1.56 nM over captured antibodies (HuNectin4-His-BirA diluted in HBS-EP+1× in ½ dilutions, injected over 120s at 40 μl/min) and allowed to dissociate for 600s. The chip was regenerated with NaOh 10 mM 2× for 30s at 40 μl/min. The sensorgram sets were fitted using the 1:1 kinetic binding model or two state reaction model, as a function of the profile of the curves.

Results are shown in the Table, below. The KD differences generally do not appear to correlate to the increased potency of 5E7 and 10B12 antibodies in ability to eliminate tumor cells. Binding affinity therefore does not explain the differences in the antibodies' potency as ADCs.

| | KD | ka | kd |
|---|---|---|---|
| | (nM) | (1/Ms) | (1/s) |
| 1 | 7.5 | 2.2E+5 | 1.7E−3 |
| 2 | 10.8 | 7.4E+4 | 8.0E−4 |
| 3 | 9.9 | 2.4E+5 | 2.4E−3 |
| 4 | 16.3 | 1.9E+5 | 3.1E−3 |
| 5 | 10.2 | 1.0E+5 | 1.0E−3 |
| 5E7 | 4.8 | 6.2E+5 | 3.0E−3 |
| 10B12 | 12.0 | 8.6E+5 | 1.0E−2 |
| N4.1 | 1.5 | 5.8E+5 | 8.7E−4 |
| Enfortumab | 13.7 | 3.4E+5 | 4.7E−3 |
| (ASG-22ME) | | | |

### Example 14: Epitope Mapping by Competition for Binding to Nectin-4 by SPR

Antibodies of Example 13, together with previously reported antibodies N4.1 (N41), antibody 14A5 and enfortumab, were tested for their ability to compete with one another for binding to the wild-type human Nectin-4 protein by SPR (Surface Plasmon Resonance) methods, with OCTET analysis using Ni-NTA (NTA) Biosensors (Fortebio). Briefly, Human Nectin4-His-BirA protein diluted to 5 μg/mL in Kinetic buffer 10× were captured onto the biosensors. The first antibody was injected diluted to 10 μg/mL in Kinetic buffer 10×, followed by a second injection of the first antibody diluted to 10 μg/mL in Kinetic buffer 10× in order to saturate the signal. The second antibody was injected diluted to 10 μg/mL in Kinetic buffer 10×.

Results are shown in the table below. Squares with a plus (+) sign indicate substantial or direct competition between the 1st and 2nd antibody (1st antibody prevents/causes loss of binding of 2nd antibody), squares with an “X” indicate potential partial competition (1st antibody causes a potential reduction but not loss of binding of 2nd antibody), white squares indicate no competition. Antibodies 5E7 and 10B12 competed with one another for binding to Nectin-4. However, there was no competition between either 5E7 or 10B12 with any of the other antibodies, other than a potential partial reduction in binding observed with the antibodies that bind the Ig-like V domain. The anti-Ig-like C₂ type 2 domain antibodies (mAbs1-5) competed with one another for binding to Nectin-4, as did the anti-Ig-like V domain antibodies.

| | | 2^{nd} mAb injected | | | | | | | | | | |
|---|---|---|---|---|---|---|---|---|---|---|---|---|
| | | 5E7 | mAb1 | mAb2 | mAb3 | mAb4 | 10B12 | mAb5 | enfortumab | N41 | 14A5 | MABT64 |
| 1^{st} | 5E7 | + | | | | | + | | | | | |
| mAb | mAb1 | | + | + | + | + | | + | | | | |
| injected | mAb2 | | + | + | + | + | | + | | | | |
| | mAb3 | | + | + | + | + | | + | | | | |
| | mAb4 | | + | + | + | + | | + | | | | |
| | 10B12 | + | | | | | + | | | | | |
| | mAb5 | | + | + | + | + | | + | | | | |
| | Enfortumab | | | | | | | | + | | | |
| | N41 | | | | | | | | | + | + | + |
| | 14A5 | | | | | | | | | + | + | + |
| | MABT64 | | | | | | | | | + | + | + |

### Example 15: Epitope Mapping of Antibodies Using Nectin-4 Point Mutants

Cell surface expressed human Nectin-4 point mutants

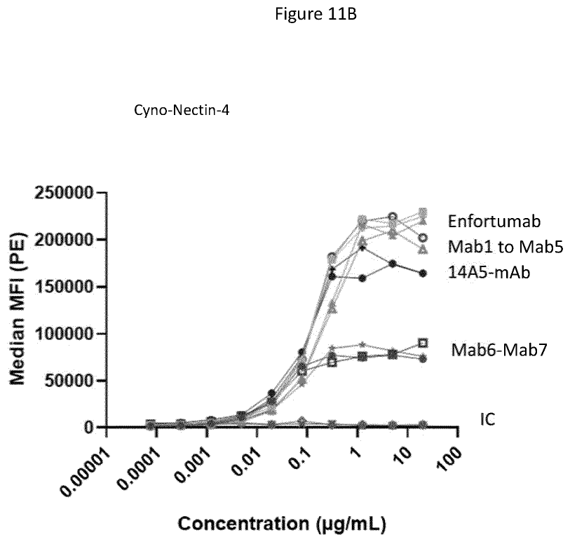

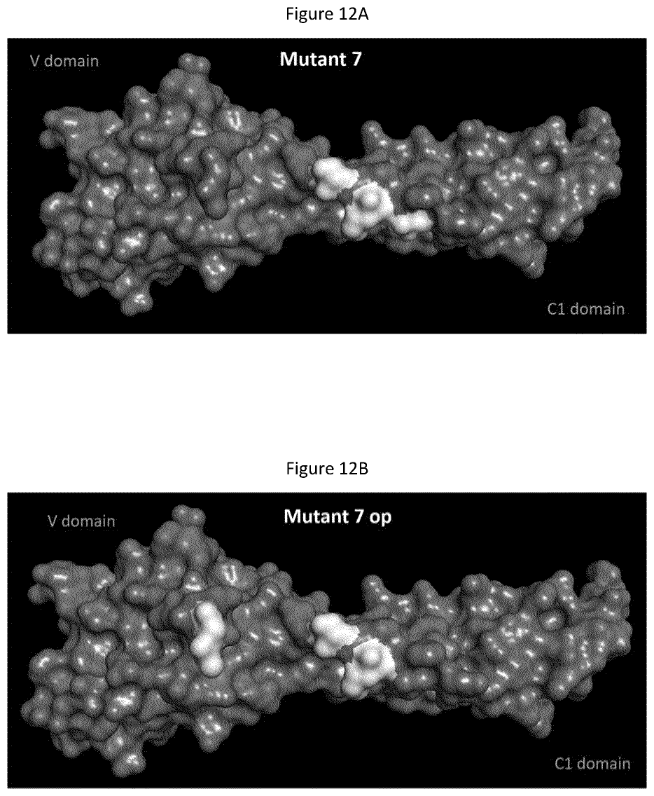

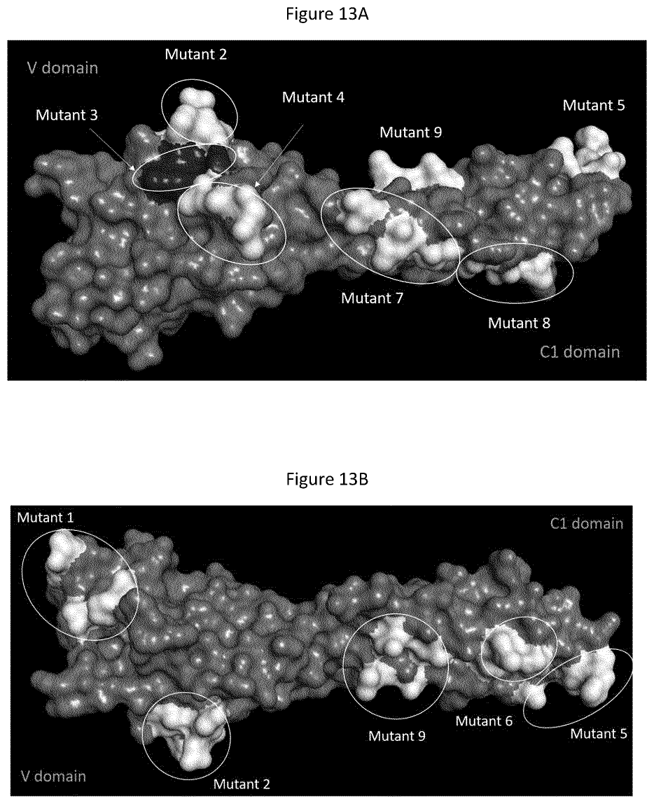

The binding profile of the anti-Nectin-4 antibodies on full-length and Ig-like V domain-deleted proteins, together with species binding profile of the antibodies (binding to human, cynomolgus and rat Nectin-4 but not to mouse Nectin-4), together with inter-species differences among the non-human Nectin-4 proteins permitted the identification of residues the junction of the Ig-like V domain and the Ig-like C₂ type 1 domain (also referred to as “C₁”). Combined with the published structures of the Nectin-4 domains, Nectin-4 mutations at surface exposed amino acids residues were designed. Nectin-4 domain structure was modelled based on Protein Data Bank reference: 4FRW (domains V et C₁). Human Nectin-4 used was NCBI Reference Sequence: NP_112178.2, mouse Nectin-4 used was NCBI Reference Sequence: AAL79833.1, cynomolgus Nectin-4 used was NCBI Reference Sequence: XP_005541277.1, rat Nectin-4 used was NCBI Reference Sequence: NP_001102546.1. The cells expressing the Nectin-4 mutants are then be used for testing anti-Nectin-4 antibodies for loss of binding to different Nectin-4 mutants to identify antibodies that bind to the same site on Nectin-4. In particular, mutants having C₁-V junction substitutions at residues K197T and/or S199A, or mutants 7, 7bis and 9 having additional and/or adjacent substitutions at the junction of domain C₁ and V, can identify antibodies having advantageous application as immunoconjugates. Mutant 7 had substitutions S195A/K197T/S199A. Mutant 7bis had substitutions A72P/G73N/K197T/S199A. Mutant 9 included the key residue Q234 substitution, and had substitutions L150S/S152A/Q234R/1236S. FIGS. 12A and 12B show a molecular model of the human Nectin-4 protein, indicating the position of substituted residues in mutants 7 (12A) and in mutants 7bis (12B); these mutants are in the C₁ domain identify two sites at the junction of domain C₁ and V domain that are on opposing faces of the Nectin-4 protein. FIGS. 13A and 13B show different view of a molecular model of the human Nectin-4 protein, indicating the position of substituted residues in mutants 1, 2, 3, 4, 5, 6, 7, 8 and 9.

Nectin-4 mutants were generated by PCR. The sequences amplified were run on agarose gel and purified using the Macherey Nagel PCR Clean-Up Gel Extraction kit. The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO₂ incubator for 48 hours prior to testing for transgene expression. Mutants were transfected in HEK-293T cells, as shown in the table below. The targeted amino acid mutations are shown in Table 2 below, listing the residue present in wild-type Nectin-4/position of residue/residue present in mutant Nectin-4, with position reference being to the Nectin-4 protein with leader peptide shown in SEQ ID NO: 1.

TABLE 2

| | Amino acid substitutions with reference |
|---|---|
| Mutant | to Nectin-4 of SEQ ID NO: 1 |
| 1 | S58P/G59D/Q61A/N106D/P107A |
| 2 | V90S/P92A/A93S/E95A/G96D |
| 3 | A76S/Q77R/E78A/S91N |
| 4 | A72P/G73N/G75S |
| 5 | A158P/E160G/S243A/F244S |
| 6 | H238A/I239T/H241Q |
| 7 | S195A/K197T/S199A |
| 7bis | A72P/G73N/K197T/S199A |
| 8 | T190A/T191Q/S193A |
| 9 | L150S/S152A/Q234R/I236S |

Results of the association between antibodies and Nectin-4 mutant are shown in the table below. (+) means that antibodies-Nectin-4 association occurs. (−) means antibodies-Nectin-4 association does not occur. Results related with mutant 5 are irrelevant, due to an absence of expression of said protein.

TABLE 3

| | EXPI | | | | | | | | | | |
|---|---|---|---|---|---|---|---|---|---|---|---|
| | medium | | | | | | | | | | |
| | (negative | Mutant | Mutant | Mutant | Mutant | Mutant | Mutant | Mutant | Mutant | Mutant | Mutant |
| | control) | 1 | 2 | 3 | 4 | 5 | 6 | 7 | 7bis | 8 | 9 |
| CHG1-M-1N4-5E7 | − | + | + | + | + | − | + | − | − | + | + |
| CHG1-M-4N4-6A7 | − | + | + | + | + | − | + | − | − | + | + |
| CHG1-M-1N4-10B12 | − | + | + | + | + | − | + | − | − | + | + |
| CHG1-M-4N4-6C11B | − | + | + | − | + | − | + | + | + | + | + |
| CHG1-M-4N4-1B7A | − | + | − | − | + | − | + | + | + | + | + |
| Enfortumab | − | + | − | − | + | − | + | + | + | + | + |
| N41 | − | − | + | + | + | − | + | + | + | + | + |
| 14A5.2 | − | − | + | + | + | − | + | + | + | + | + |

Antibodies 10B12, 5E7 and 6A7 therefore have a binding site on Nectin-4 encompassing C₁ domain residues that are mutated in Mutant 7 and 7bis (S195A/K197T/S199A and A72P/G73N/K197T/S199A). Antibody 6C₁₁B has a binding site on Nectin-4 encompassing V domain residues that are mutated in Mutant 3 (A76S/Q77R/E78A/S91N). Antibody 1B7A has a binding site on Nectin-4 encompassing V domain residues that are mutated in mutant 2 (V90S/P92A/A93S/E95A/G96D) and mutant 3 (A76S/Q77R/E78A/S91N).

Thus, antibodies 10B12, 5E7 and 6A7 bind epitopes on Nectin-4 that are different than Enfortumab, N41 and 14A5 (that bind epitopes on the V domain of Nectin-4). Although 6C₁₁B and enfortumab bind the same region of Nectin-4 on, their epitopes are different.

### Example 16: A Second Immunization and Directed Screen for Anti-VC1 Domain Antibodies

In order to obtain further antibodies having the same or similar functional properties as antibodies 5E7 and 10B12, a further immunization was using the methods of Example 4. Balb/c mice were immunized with recombinant human Nectin-4 protein having the full-length wild-type Nectin-4 amino acid sequence fused to a C-terminal 6-His tag, followed by determination of the titers of each sera on a CHO cell line made to express at its surface full-length human Nectin-4 to select the best responder mice for fusion.

Hybridomas were grown and supernatants were harvested and subjected to screening for binding to Nectin-4 by flow cytometry in two successive screens (screening #1 and confirmation screening #2), using the wild-type Nectin-4 expressing CI.3C₈ cells described above, with antibody 5E7 used as positive control and isotype controls as negative controls. These hybridomas were amplified, supernatants were harvested, and cells were frozen for preservation of RNAs.

As in Example 4, by this method, two groups of titrations were observed on the CHO cell line expressing wild-type Nectin-4, the first group with a majority of antibodies with a higher plateau and a second profile with a lower plateau represented by one antibody having an IC₅₀ substantially the same as 5E7.

Selected hybridomas were further characterized by determining the species cross-reactivity on human, rat, mouse and cynomolgus Nectin-4, as well as binding to Nectin-4 domain-substituted proteins as in Example 4. The new antibodies were assessed for their ability to bind different domains on human Nectin-4 by flow cytometry using the wild-type human Nectin-4 cell line containing the whole Nectin-4 protein and the human Nectin4-C1C2-V5 sorted cell line.

Of the antibodies that titrated on the Nectin-4 expressing CI.3C₈ cells (IC₅₀ values observed were comparable to 5E7), six antibodies bound the Ig-like V domain, displaying binding to the whole Nectin-4 protein with loss of all binding to the C1C2 construct, seven antibodies were anti-Ig-like C₂ type 1 domain binders, displaying full binding to the whole Nectin-4 protein as well as to both the C₁ (having only C₂ type 1 domain) and C1C2 construct (having only C₂ type 1 and C₂ type 2 domains). Two antibodies (clones 6C₁₁B and 6A7) showed a partial loss of binding to wild-type Nectin-4 and retained binding to the C1C2 construct, along with loss of binding to the C₁ construct suggesting that they bind Nectin-4 partially on the V domain and partially on the Ig-like C₂ type 1 domain.

The anti-Nectin-4 antibodies were tested by flow cytometry for binding to different CHO cell lines made to express respectively the mouse, cynomolgus and rat Nectin-4 protein according to the methods in Example 11. Antibodies 6C₁₁B and 6A7 bound to rat and cynomolgus Nectin-4 protein. The anti-Nectin-4 antibodies were further tested by flow cytometry for binding to different CHO cell lines made to express respectively the human

Nectin 1, Nectin 2, Nectin 3 and PVR proteins as in Example 12. Antibodies 6C₁₁B and 6A7 did not bind any of respectively the human Nectin 1, Nectin 2, Nectin 3 and PVR proteins. Epitope mapping was carried out by assessing competition for binding to Nectin-4 by SPR as in Example 14. Antibody 6A7 competed with 5E7 for binding to human Nectin-4 while antibody 6C₁₁B did not compete with 5E7 for binding to human Nectin-4. 5E7 and 6A7 identify an epitope bin different from that of 6C₁₁B, which may be explained by the binding by the antibodies to Nectin-4 at the junction of domain C₁ and V on different or opposite faces of the Nectin-4 protein. FIG. 12, discussed above, shows an epitope bin on a first face of Nectin-4 involving one or more of the residues substituted in mutant 7 and another epitope bin on a second face of Nectin-4 involving one or more of the residues substituted in mutant 9.

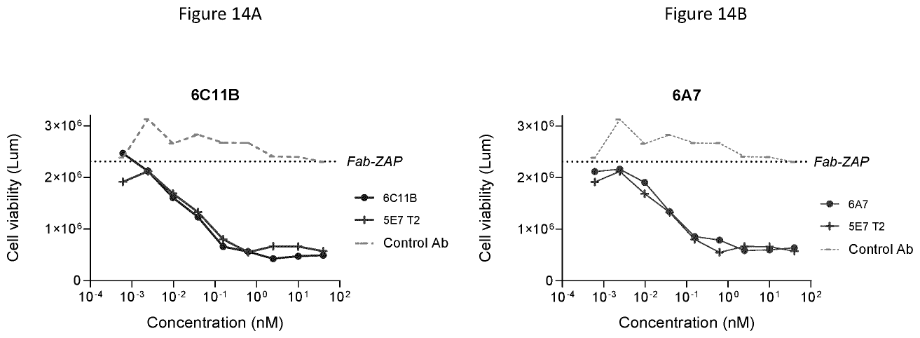

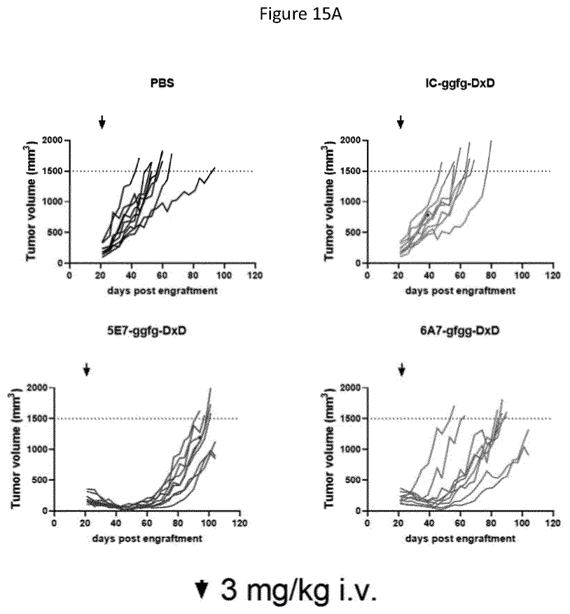

The ability of the anti-nectin-4 antibodies to induce nectin-4 internalization was assessed on SUM190 cell lines using the Fab-ZAP human Internalization Kit (Advanced Targeting Systems), with measurement of cell viability using the CTG substrate (CellTiter-Glo® Luminescent Cell Viability Assay (Promega), as in Example 6. FIGS. 14A and 14B show internalization (cell viability measured as luminescence) compared to 5E7 for each of antibodies 6C₁₁B and 6A7, respectively, showing that 6C₁₁B and 6A7 both show a strong ability to induce intracellular internalization of Nectin-4 on tumor cells.

### Example 17: In Vivo Efficacy of Anti-Nectin 4 Antibody-Drug Conjugate (ADC)

Human breast cancer cell line SUM190PT (BioIVT 28068A1-6281) was cultured in the following cell culture medium containing Ham's F12, FBS 1 g/L, HEPES 10 mM, Ethanolamine 5 mM, Insulin 5 μg/mL, Hydrocortisone 1 μg/mL, Apo-Transferrin 5 μg/mL, Triiodo Thyronine (T3) 6.7 ng/ml, Sodium selenite 8.7 ng/ml.

Immunodeficient CB17—SCID mice were used at 7 to 8 weeks of age. Anti-Nectin-4 antibodies enfortumab, 5E7, 6A7, 6C₁₁B and 1B7A having a human Fc region were produced and conjugated to payloads Dxd (deruxtecan) or Exatecan via intracellularly cleavable linkers ggfg-Dxd or PEG (8U)-Val-Ala-PAB-Exatecan. The ggfg-Dxd linker will release Dxd (deruxtecan) upon cleavage and the PEG (8U)-Val-Ala-PAB-Exatecan linker will release exatecan upon cleavage.

SUM190 cells were subcutaneously engrafted in CB17—SCID immunodeficient mice at the dose of 0.5 million in 100 μl of Matrigel containing growth factor diluted at ½ in PBS. At day 21, when tumors reached a mean volume at 146.9±63.2 mm3 or 213.2±77.5 mm3 depending on the experiment, mice were randomized into groups of 8 or 9 mice depending on the experiment and treated intravenously with a single injection of 3 or 10 mg/kg body weight ADC or PBS as control. Tumor growths were followed twice a week. Kaplan Meier survival curves were established by using GraphPad Prism V7 software according to the following criteria: when the tumor volume reached 1500 mm3, mice were euthanized and considered dead on the day of sacrifice (D). When tumors were highly necrotic, mice were euthanized and considered dead on the same day (D).

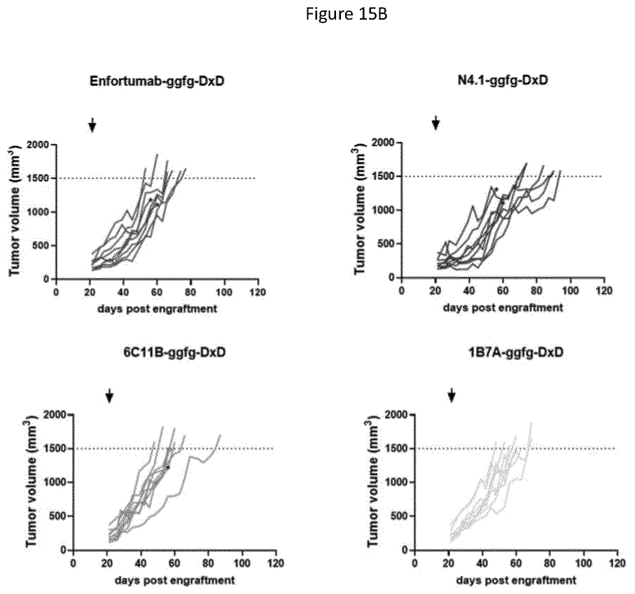

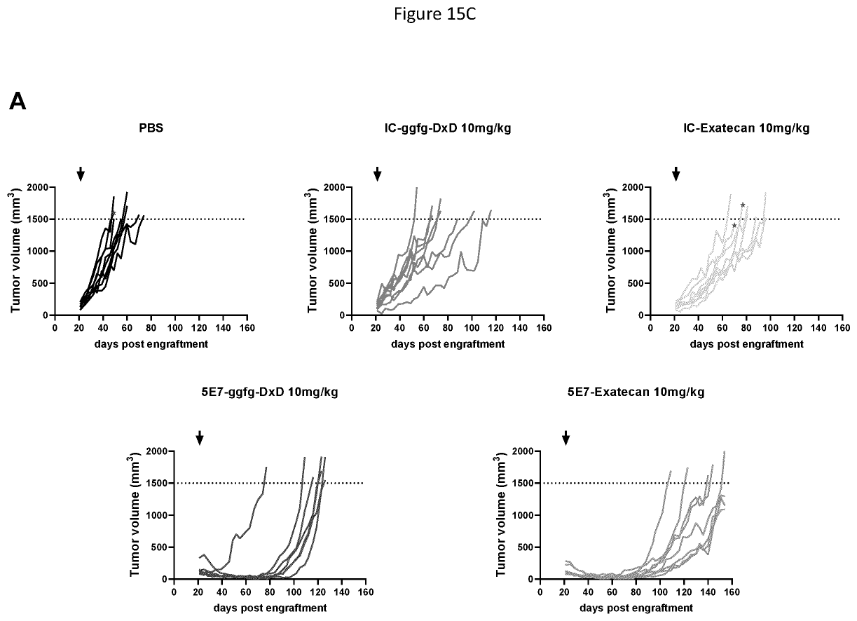

Results are presented in FIGS. 15A and 15B for the 3 mg/kg dose, where it can be seen that the anti-IgVC1 ADCs 5E7-ggfg-Dxd and 6A7-ggfg-Dxd that were found to lose binding to Nectin-4 having K197T/S199A mutations exhibit the highest efficiency in limiting the tumor growth in mice. In addition, ADCs 6C₁₁-ggfg-Dxd and 1B7A-ggfg-Dxd were somewhat more efficient than Enfortumab-ggfg-Dxd and N4. 1-ggfg-Dxd in controlling the tumor growth.

Furthermore, 5E7 conjugated with the PEG (8U)-Val-Ala-PAB-Exatecan) designed to release exatecan upon linker cleavage shows a higher efficiency in controlling tumor growth in mice compared 5E7 conjugated with ggfg-Dxd designed to release Dxd upon linker cleavage, as shown in FIG. 15C for the 10 mg/kg dose.

### Example 18: In Vitro Efficacy of ADCs in Pg-p-Expressing Cancer Model

MC-38 cells that endogenously express MDR1 P-glycoprotein (Pgp) were engineered to express Nectin-4 and cultured in DMEM+10% FBS. Cells were either treated with vehicle (DSMO) or with cyclosporin A (5 μM, stock solution in DMSO) known to act as an inhibitor of Pgp, in presence of ADCs. The ADCs tested were as follows:
(a) PADCEV™,
(b) Antibody 5E7 conjugated to deruxtecan (Dxd) via the ggfg-Dxd linker which releases Dxd upon cleavage (5E7-GGFG-DxD), and
(c) Antibody 5E7 conjugated to exatecan via the PEG (8U)-Val-Ala-PAB-Exatecan linker which releases exatecan upon cleavage (5E7-exatecan).

The ADCs and the equivalent isotype control ADCs were used at dose range from 150 to 2.3 10⁻³ nM. After five days of co-incubation with cells, cell viability was measured by addition of Cell Titer Glo™ (CTG) substrate. Luminescence vs. Ab concentration was plotted.

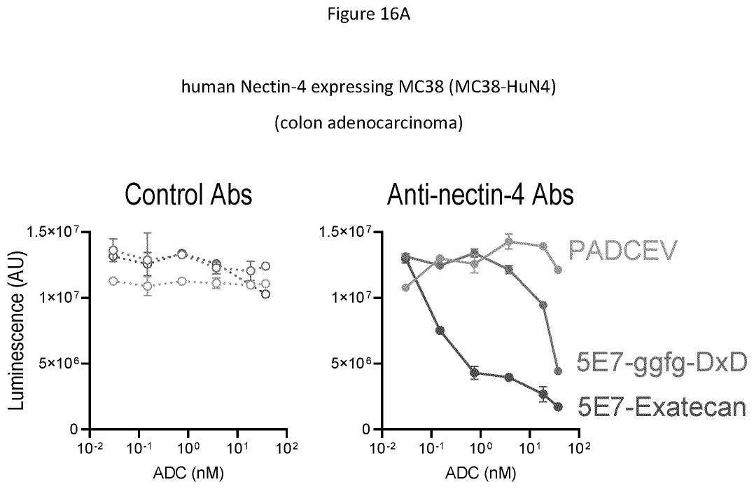

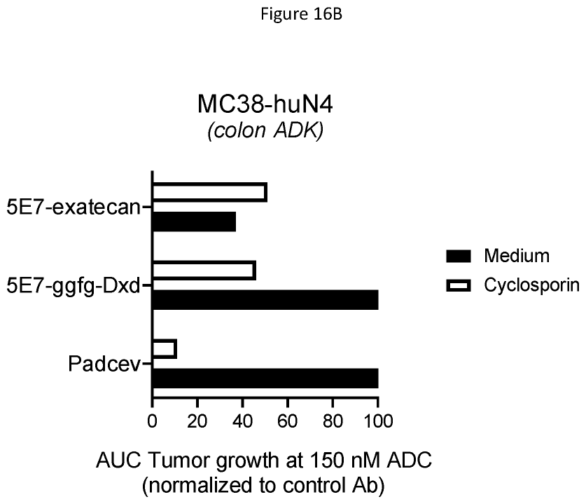

FIG. 16A shows luminescence (indicating cell viability) of cells treated with Padcev™ (enfortumab vedotin), antibody 5E7 conjugated to Dxd or 5E7 conjugated to exatecan. The ADC with exatecan (5E7-exatecan) as payload was highly potent to decrease cell viability in this setting of drug resistance. FIG. 16B shows tumor growth (area under the curve) of the MC38 cells treated, in the presence or absence of the Pgp inhibitor cyclosporine, with Padcev™ (enfortumab vedotin), antibody 5E7 conjugated to Dxd or 5E7 conjugated to exatecan, at 150 nM ADC and normalized to control antibody. The results suggest that the anti-tumor activity of Padcev™ and antibody 5E7 conjugated to Dxd are negatively affected by Pgp.

### Example 19: In Vivo Efficacy of ADCs in Pg-p-Expressing Cancer Model

MC-38 cells that endogenously express MDR1 P-glycoprotein were engineered to express Nectin-4 and cultured. On day 1 of the experiment, MC-38 cells were subcutaneously injected in Immunodeficient CB17—SCID mice were used at 7 to 8 weeks of age at the dose of 0.5 million cells in 100 μl of Matrigel containing growth factor diluted at ½ in PBS. ADCs tested in this experiment were as follows:
(a) PADCEV™,
(b) Antibody 6A7 conjugated to deruxtecan (Dxd) via the ggfg-Dxd linker which releases Dxd upon cleavage (6A7-deruxtecan), and
(c) Antibody 6A7 conjugated to exatecan via the PEG (8U)-Val-Ala-PAB-Exatecan linker which releases exatecan upon cleavage (6A7-exatecan).

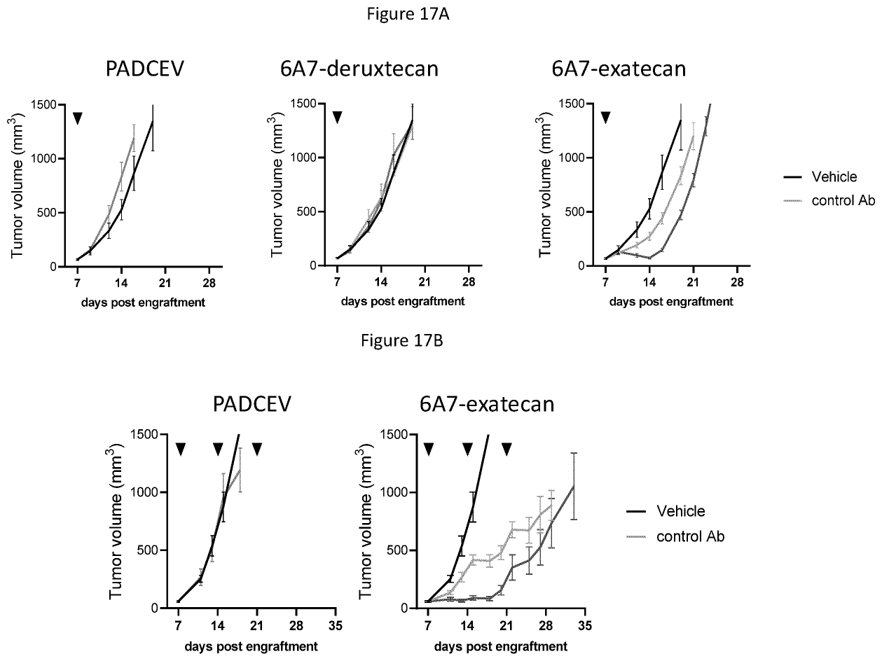

In a first experiment, a single dose of 10 mg/kg of enfortumab vedotin (PADCEV™), or antibody 6A7 conjugated to Exatecan or deruxtecan was intravenously injected to each mouse at day 7. In a second experiment, three doses of 10 mg/kg of enfortumab vedotin (PADCEV™) or 6A7 conjugated to exatecan were intravenously injected to each mouse at day 7, 14 and 21. Tumor volume in mice was followed from the first injection. FIGS. 17A (single dose) and 17B (three dose) present the mean tumor volume over time, until occurrence of two deaths per group. FIG. 17A also shows that a single injection of 6A7 conjugated to exatecan (right-most curve in the right panel) induces a significantly higher reduction of tumor growth compared to a single injection of enfortumab vedotin or a single injection of 6A7 conjugated to deruxtecan. In addition, FIG. 17B shows that three successive weekly injections of 6A7 conjugated to exatecan have a greater anti-tumor effect compared to three weekly injections of enfortumab vedotin.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1-70. (canceled)

71. A method for making an antibody-drug conjugate (ADC), optionally wherein the ADC is for use in the treatment of a cancer, the method comprising conjugating a cytotoxic agent (Z) to an anti-Nectin-4 antibody, wherein the anti-Nectin-4 antibody binds to the VC1 bridging domain of a human Nectin-4 polypeptide.

72. The method of claim 71, wherein the cytotoxic agent (Z) is exatecan.

73. The method of claim 71, wherein the antibody binds to the Ig-like V domain and to the Ig-like C₁ type 2 domain of Nectin-4.

74. The method of claim 71, wherein the antibody binds to an epitope comprising amino acid residues K197 and/or S199 of Nectin-4 (with reference to SEQ ID NO: 1), and/or wherein the antibody has reduced binding to a mutant Nectin-4 polypeptide comprising a mutation at residues K197 and/or S199, relative to binding between the antibody and a wild-type Nectin-4 polypeptide comprising the amino acid sequence of SEQ ID NO: 1.

75. The method of claim 71, wherein the antibody is a function conservative variant of antibody 5E7, 10B12, 6C₁₁B or 6A7.

76. The method of claim 71, wherein the antibody comprises:
(a) a V_{H} comprising the Kabat CDR1, CDR2 and/or CDR3 of the heavy chain variable region having the amino acid sequence of SEQ ID NO: 6 and a V_{L} comprising a Kabat CDR1, CDR2 and/or CDR3 of the light chain variable region having the amino acid sequence of SEQ ID NO: 7;
(b) a V_{H} comprising the Kabat CDR1, CDR2 and/or CDR3 of the heavy chain variable region having the amino acid sequence of SEQ ID NO: 24 and a V_{L} comprising a Kabat CDR1, CDR2 and/or CDR3 of the light chain variable region having the amino acid sequence of SEQ ID NO: 25;
(c) a V_{H} comprising the Kabat CDR1, CDR2 and/or CDR3 of the heavy chain variable region having the amino acid sequence of SEQ ID NO: 42 and a V_{L} comprising a Kabat CDR1, CDR2 and/or CDR3 of the light chain variable region having the amino acid sequence of SEQ ID NO: 43; or
(d) a V_{H} comprising the Kabat CDR1, CDR2 and/or CDR3 of the heavy chain variable region having the amino acid sequence of SEQ ID NO: 50 and a V_{L} comprising a Kabat CDR1, CDR2 and/or CDR3 of the light chain variable region having the amino acid sequence of SEQ ID NO: 51.

77. A method of treating a cancer, killing tumor cells and/or sensitizing a tumor to a cytotoxic agent, in an individual in need thereof, comprising administering to said individual a therapeutically effective amount of an anti-Nectin-4 antibody that binds to the VC1 bridging domain of a human Nectin-4 polypeptide, in combination with a cytotoxic agent, optionally wherein the cytotoxic agent is administered separately on the same day or on a different day.

78. A method of treating a cancer, killing tumor cells and/or delivering a cytotoxic agent to a tumor, in an individual in need thereof, comprising administering to said individual a therapeutically effective amount of an antibody-drug conjugate comprising an anti-Nectin-4 antibody conjugated via a linker to a cytotoxic agent (Z), wherein the antibody binds to the VC1 bridging domain of a human Nectin-4 polypeptide.

79. The method according to claim 78, wherein the cytotoxic agent (Z) is a camptothecin, optionally a five-ring camptothecin analogue, optionally a six-ring camptothecin analogue, optionally exatecan, SN-38 or Dxd and wherein said individual has a cancer that has relapsed and/or that has progressed following treatment with enfortumab verdotin.

80. A method of treating a cancer, killing tumor cells and/or delivering a cytotoxic agent to a tumor, in an individual in need thereof, comprising administering to said individual an antibody-drug conjugate comprising an anti-Nectin-4 antibody conjugated via a linker to a cytotoxic agent, in combination with an antibody-drug conjugate comprising an anti-HER2 antibody conjugated via a linker to a cytotoxic agent, wherein at least one of the anti-Nectin-4 and anti-HER2 is represented by the formula:
Ab-(X—(Z))
wherein
Ab is a polypeptide, peptide or antibody that specifically binds to a human Nectin-4 polypeptide or a human HER2 polypeptide, respectively,
X is a molecule which connects Ab and Z, wherein X comprises a cleavable moiety, e.g., under physiological conditions, optionally under intracellular conditions, optionally a protease-cleavable di-, tri-, tetra- or penta-peptide, and
Z is exatecan.

81. An immunoconjugate that binds a human Nectin-4 polypeptide,
represented by Formula (I):
Ab-(X—(Z)) Formula (I)
wherein,
Ab is a polypeptide, peptide, antibody that specifically binds to the VC1 bridging domain of a human Nectin-4 polypeptide;
X is a molecule which connects Ab and Z, wherein X comprises cleavable moiety, e.g., under physiological conditions, optionally under intracellular conditions, optionally a protease-cleavable di-, tri-, tetra- or penta-peptide; and
Z comprises a cytotoxic agent, optionally a camptothecin analogue, optionally a five-ring camptothecin analogue, optionally a six-ring camptothecin analogue, optionally exatecan, SN-38 or Dxd.

82. An antibody that binds to a human Nectin-4 polypeptide, wherein the antibody comprises:
(a) a heavy chain variable region (V_{H}) comprising the heavy chain CDR 1, 2 and 3 of the heavy chain variable region of SEQ ID NO: 6 and a light chain variable region (V_{L}) comprising the light chain CDR 1, 2 and 3 of the light chain variable region of SEQ ID NO: 7;
(b) a V_{H} comprising the Kabat CDR1, CDR2 and/or CDR3 of the heavy chain variable region having the amino acid sequence of SEQ ID NO: 24 and a V_{L} comprising a Kabat CDR1, CDR2 and/or CDR3 of the light chain variable region having the amino acid sequence of SEQ ID NO: 25;
(c) a V_{H} comprising the Kabat CDR1, CDR2 and/or CDR3 of the heavy chain variable region having the amino acid sequence of SEQ ID NO: 42 and a V_{L} comprising a Kabat CDR1, CDR2 and/or CDR3 of the light chain variable region having the amino acid sequence of SEQ ID NO: 43; or
(d) a V_{H} comprising the Kabat CDR1, CDR2 and/or CDR3 of the heavy chain variable region having the amino acid sequence of SEQ ID NO: 50 and a V_{L} comprising a Kabat CDR1, CDR2 and/or CDR3 of the light chain variable region having the amino acid sequence of SEQ ID NO: 51.

83. A nucleic acid or set of nucleic acids encoding a heavy and/or light chain of an antibody of claim 82.

84. A hybridoma or recombinant host cell producing the antibody of claim 82.

85. A method of treating a cancer, killing tumor cells and/or delivering a cytotoxic agent to a tumor, in an individual in need thereof, the method comprising administering to said individual a therapeutically effective amount of an antibody-drug conjugate comprising an anti-Nectin-4 antibody conjugated via a cleavable linker (and optionally a self-eliminating spacer) to an exatecan of Compound 1, and wherein said individual has cancer that has relapsed and/or that has progressed following treatment with a binding agent (optionally a Nectin-4-binding agent or a HER2 binding agent) conjugated via a linker to a cytotoxic agent capable of being transported by Pgp.

86. A method of testing an antibody that binds the VC1 bridging domain of Nectin-4-binding, the method comprising: (i) bringing the antibody into contact with a cell expressing at its surface Nectin-4, optionally wherein the cell expresses Pgp, in the presence of cytotoxic agent, optionally wherein the antibody is conjugated to the cytotoxic agent, and (ii) assessing the ability of the antibody or conjugated antibody to cause death of the cell, optionally assessing whether the antibody or conjugated antibody in the presence of cytotoxic agent increases cell death compared to that observed in presence of the cytotoxic agent alone.


SEQUENCE LISTING (57 sequences)

SEQ ID NO: 1 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 2 - protein, 325 aa, Homo sapiens
GRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVV

SEQ ID NO: 3 - protein, 113 aa, Homo sapiens
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLR

SEQ ID NO: 4 - protein, 90 aa, Homo sapiens
PPLPSLNPGPALEEGQGLTLAASCTAEGSPAPSVTWDTEVKGTTSSRSFKHSRSAAVTSEFHLVPSRSMNGQPLTCVVSHPGLLQDQRIT

SEQ ID NO: 5 - protein, 103 aa, Homo sapiens
ASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASV

SEQ ID NO: 6 - protein, 117 aa, Mus musculus
QVQLQQPGAELVKPGASVKLSCKASGYIFTSYWMHWVKQRPGQGLEWIGEIDPSDSYTNYNQKFKGKATLTLDKSSSTTYMQLSSLTSEDSAVYYCVRGYGNYGDYWGQGTTLTVSS

SEQ ID NO: 7 - protein, 112 aa, Mus musculus
DVVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 8 - protein, 5 aa, Mus musculus
SYWMH

SEQ ID NO: 9 - protein, 17 aa, Mus musculus
EIDPSDSYTNYNQKFKG

SEQ ID NO: 10 - protein, 8 aa, Mus musculus
GYGNYGDY

SEQ ID NO: 11 - protein, 16 aa, Mus musculus
RSSKSLLHSNGITYLY

SEQ ID NO: 12 - protein, 7 aa, Mus musculus
QMSNLAS

SEQ ID NO: 13 - protein, 9 aa, Mus musculus
AQNLELPWT

SEQ ID NO: 14 - protein, 8 aa, Mus musculus
GYIFTSYW

SEQ ID NO: 15 - protein, 8 aa, Mus musculus
IDPSDSYT

SEQ ID NO: 16 - protein, 10 aa, Mus musculus
VRGYGNYGDY

SEQ ID NO: 17 - protein, 11 aa, Mus musculus
KSLLHSNGITY

SEQ ID NO: 18 - protein, 3 aa, Mus musculus
QMS

SEQ ID NO: 19 - protein, 7 aa, Mus musculus
GYIFTSY

SEQ ID NO: 20 - protein, 4 aa, Mus musculus
PSDS

SEQ ID NO: 21 - protein, 6 aa, Mus musculus
YGNYGD

SEQ ID NO: 22 - protein, 12 aa, Mus musculus
SKSLLHSNGITY

SEQ ID NO: 23 - protein, 6 aa, Mus musculus
NLELPW

SEQ ID NO: 24 - protein, 117 aa, Mus musculus
QVQLQQPGAELVKPGASVKLSCKASGYTFTSYWMHWVKQRPGQGLEWIGEIDPSDSYTNYNQKFKGKATLTVDKSSSTAYMQLSSLTSEDSAVYYCVRGYGNYGDYWGQGTTLTVSS

SEQ ID NO: 25 - protein, 112 aa, Mus musculus
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 26 - protein, 7 aa, Mus musculus
GYTFTSY

SEQ ID NO: 27 - protein, 8 aa, Mus musculus
GYTFTSYW

SEQ ID NO: 28 - protein, 116 aa, Mus musculus
QVQLKQSGPGLVQPSQSLSITCTVSGFSLTNYGVHWVRQSPGKGLEWLGVIWSGGSTDYNAAFISRLSISKDTSKSQVFFKMNSLQADDTAIYYCARELIHAMDNWGQGTSVTVSS

SEQ ID NO: 29 - protein, 106 aa, Mus musculus
DIQMTQSPASLSVSVGETVTITCRASENIYSNLAWYQQKQGNSPQLLVFAATNLADGVPSRFSGSGSGTQYSLKINSLQSEDFGTYYCQHFWGTPTFGGGTKLEIK

SEQ ID NO: 30 - protein, 339 aa, Artificial Sequence
  synthetic
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASHHHHHHLHHILDAQKMVWNHR

SEQ ID NO: 31 - protein, 479 aa, Homo sapiens
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 32 - protein, 363 aa, Homo sapiens
PPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 33 - protein, 263 aa, Homo sapiens
ASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 34 - protein, 477 aa, Mus musculus
ELETSDVVTVVLGQDAKLPCFYRGDPDEQVGQVAWARVDPNEGIRELALLHSKYGLHVNPAYEDRVEQPPPPRDPLDGSVLLRNAVQADEGEYECRVSTFPAGSFQARMRLRVLVPPLPSLNPGPPLEEGQGLTLAASCTAEGSPAPSVTWDTEVKGTQSSRSFTHPRSAAVTSEFHLVPSRSMNGQPLTCVVSHPGLLQDRRITHTLQVAFLAEASVRGLEDQNLWQVGREGATLKCLSEGQPPPKYNWTRLDGPLPSGVRVKGDTLGFPPLTTEHSGVYVCHVSNELSSRDSQVTVEVLDPEDPGKQVDLVSASVIIVGVIAALLFCLLVVVVVLMSRYHRRKAQQMTQKYEEELTLTRENSIRRLHSHHSDPRSQPEESVGLRAEGHPDSLKDNSSCSVMSEEPEGRSYSTLTTVREIETQTELLSPGSGRTEEDDDQDEGIKQAMNHFVQENGTLRAKPTGNGIYINGRGHLV

SEQ ID NO: 35 - protein, 508 aa, Rattus rattus
MPLSLGAEMWGPEAWLLLLFLASFTGRYSAGELETSDLVTVVLGQDAKLPCFYRGDPDEQVGQVAWARVDPNEGTRELALLHSKYGLHVSPAYEDRVEQPPPPRDPLDGSILLRNAVQADEGEYECRVSTFPAGSFQARMRLRVLVPPLPSLNPGPPLEEGQGLTLAASCTAEGSPAPSVTWDTEVKGTQSSRSFKHSRSAAVTSEFHLVPSRSMNGQPLTCVVSHPGLLQDQRITHTLQVAFLAEASVRGLEDQNLWHVGREGATLKCLSEGQPPPKYNWTRLDGPLPSGVRVKGDTLGFPPLTTEHSGVYVCHVSNELSSRASQVTVEVLDPEDPGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRTEEEDDQDEGIKQAMNHFVQENGTLRAKPTGNGIYINGRGHLV

SEQ ID NO: 36 - protein, 479 aa, Canis lupus
GELETSDVVTVVLGQDAKLPCFYRGDSGEQVGQVAWARADAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHVGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRTEEEEDQDEGIKQAMNHFVQENGTLRAKPTGNGIYINGRGHLV

SEQ ID NO: 37 - protein, 514 aa, Homo sapiens
MGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 38 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 39 - protein, 549 aa, Homo sapiens
MARTLRPSPLCPGGGKAQLSSASLLGAGLLLQPPTPPPLLLLLFPLLLFSRLCGALAGPIIVEPHVTAVWGKNVSLKCLIEVNETITQISWEKIHGKSSQTVAVHHPQYGFSVQGEYQGRVLFKNYSLNDATITLHNIGFSDSGKYICKAVTFPLGNAQSSTTVTVLVEPTVSLIKGPDSLIDGGNETVAAICIAATGKPVAHIDWEGDLGEMESTTTSFPNETATIISQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTFNYSGVYICKVTNSLGQRSDQKVIYISDPPTTTTLQPTIQWHPSTADIEDLATEPKKLPFPLSTLATIKDDTIATIIASVVGGALFIVLVSVLAGIFCYRRRRTFRGDYFAKNYIPPSDMQKESQIDVLQQDELDSYPDSVKKENKNPVNNLIRKDYLEEPEKTQWNNVENLNRFERPMDYYEDLKMGMKFVSDEHYDENEDDLVSHVDGSVISRREWYV

SEQ ID NO: 40 - protein, 390 aa, Homo sapiens
DVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 41 - protein, 14 aa, Artificial Sequence
  Simian virus 5
GKPIPNPLLGLDST

SEQ ID NO: 42 - protein, 120 aa, Mus musculus
EVQLQQSGPELVKPGASVKISCKTSGYTFTEYTIHWVKQNHGKSLEWIGRINPKNGDIIHNQNFMGKATLTVDKSSSTAYMELRSLTSEDSAVCYCARSGYGSSYGFAYWGQGTLVTVSA

SEQ ID NO: 43 - protein, 111 aa, Mus musculus
DIVMTQSPTSLVVSLGQRATISCRASQSVTTPRYSYMHWYQQKPGQPPKLLIKYASNLESGVPARFSGSGSGTDFTLNIHPVEEEDTATYYCQHSWEIPYTFGGGTKLEIK

SEQ ID NO: 44 - protein, 5 aa, Mus musculus
EYTIH

SEQ ID NO: 45 - protein, 17 aa, Mus musculus
RINPKNGDIIHNQNFMG

SEQ ID NO: 46 - protein, 11 aa, Mus musculus
SGYGSSYGFAY

SEQ ID NO: 47 - protein, 15 aa, Mus musculus
RASQSVTTPRYSYMH

SEQ ID NO: 48 - protein, 7 aa, Mus musculus
YASNLES

SEQ ID NO: 49 - protein, 9 aa, Mus musculus
QHSWEIPYT

SEQ ID NO: 50 - protein, 118 aa, Mus musculus
QVQLKESGAELVKPGASVKLSCKASGYTFTSYWMHWVKQRPGHGLEWVGEIDPSDSYTNYNQKFKGKATLTIDKSSSTVYMQLSSLTSEDSAVYYCARSGNYDAMDYWGQGTSVTVSA

SEQ ID NO: 51 - protein, 112 aa, Mus musculus
DIVMTQAAFSSAVTLGTSASISCRSSKSLLHSNGITYLYWYLQKPGQSPHFLIYQMSNLASGVPDRFTSSGSGTDFTLRISRVEAEDVGVYYCAQNLELPWTFGGGTKLEIK

SEQ ID NO: 52 - protein, 5 aa, Mus musculus
SYWMH

SEQ ID NO: 53 - protein, 17 aa, Mus musculus
EIDPSDSYTNYNQKFKG

SEQ ID NO: 54 - protein, 9 aa, Mus musculus
SGNYDAMDY

SEQ ID NO: 55 - protein, 16 aa, Mus musculus
RSSKSLLHSNGITYLY

SEQ ID NO: 56 - protein, 7 aa, Mus musculus
QMSNLAS

SEQ ID NO: 57 - protein, 9 aa, Mus musculus
AQNLELPWT